(19) United States
(12) Patent Application Publication
(10) Pub. No.: US 2012/0289501 A1
(43) Pub. Date: Nov. 15, 2012

(54) Benzene-fused 6-membered oxygen-containing heterocyclic derivatives of bicyclic heteroaryls

(75) Inventors: Bei Chen, San Diego, CA (US); Robin Alec Fairhurst, Riehen (CH); Andreas Floersheimer, Dornach (CH); Pascal Furet, Thann (FR); Songchun Jiang, San Diego, CA (US); Wenshuo Lu, San Diego, CA (US); Thomas H. Marsilje, III, San Diego, CA (US); Andrea Vaupel, Riehen (CH)

(73) Assignee: NOVARTIS AG, Basel (CH)

(21) Appl. No.: 13/511,564

(22) PCT Filed: Nov. 23, 2010
(86) PCT No.: PCT/EP2010/068038

§ 371 Date: Jul. 12, 2012

Related U.S. Application Data
(60) Provisional application No. 61/264,482, filed on Nov. 25, 2009.

(51) Int. Cl. A61K 31/519; A61K 31/4985; A61P 27/02; C07D 495/08; A61P 35/00; A61P 11/00; C07D 487/04; A61K 31/541

(52) U.S. Cl. 514/228.5; 544/280; 514/265.1; 544/350; 514/249; 544/58.2; 514/252.16; 544/230

(57) ABSTRACT

The invention relates to new derivatives of formula I, wherein the substituents are as defined in the specification; to processes for the preparation of such derivatives; pharmaceutical compositions comprising such derivatives; such derivatives as a medicament; such derivatives for the treatment of a proliferative disease.

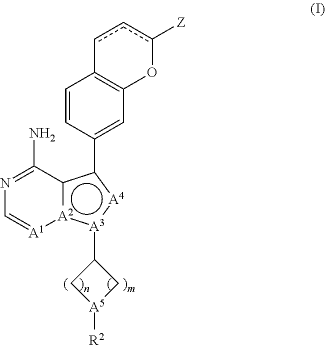

The invention relates to new benzene-fused 6-membered oxygen-containing heterocyclic derivatives of bicyclic heteroaryls; processes for the preparation of such derivatives; pharmaceutical compositions comprising such derivatives optionally in combination with one or more other pharmaceutically active compounds; such derivatives optionally in combination with one or more other pharmaceutically active compounds as a medicament; such derivatives optionally in combination with one or more other pharmaceutically active compounds for the treatment of a proliferative disease, such as a tumour disease (also including a method for the treatment of such diseases in mammals, especially in humans); and the use of such derivatives for the preparation of a pharmaceutical composition (medicament) for the treatment of a proliferative disease, such as a tumour.

Insulin-like growth factor (IGF-1) signaling is typically implicated in cancer, with the IGF-1 receptor (IGF-1R) as the predominating factor. IGR-1R is typically important for tumor transformation and survival of malignant cells, but is typically only partially involved in normal cell growth. Targeting of IGF-1R has been suggested to be a promising option for cancer therapy. (Larsson et al., Br. J. Cancer 92:2097-2101 (2005)). WO 97/028161 discloses certain pyrrolopyrimidine derivatives having therapeutic activity as inhibitors of tyrosine kinases. WO 2007/115620 discloses certain pyrrolopyrimidine derivatives having therapeutic activity as kinase inhibitors. WO 2007/079164 discloses certain pyrrolopyrimidine derivatives having therapeutic activity as tyrosine kinase inhibitors.

Because of the emerging disease-related roles of IGF-1R, there is a continuing need for compounds which may be useful for treating and preventing a disease which responds to inhibition of IGF-1R, particularly for compounds with improved efficacy, tolerabilty and/or selectivity.

Surprisingly, it has now been found that the compounds of formula I, described below, are potent inhibitors of the tyrosine kinase activity of the Insulin-like growth factor I receptor (IGF-IR) and inhibit IGF-IR-dependent cell proliferation.

The invention relates in a first aspect to a compound of formula I:

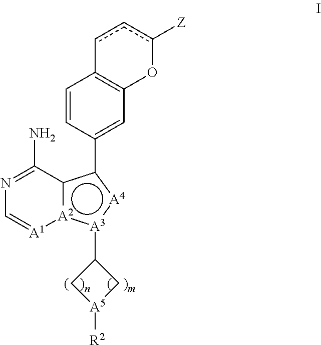

or a salt thereof, wherein [symbol]
represents an optional double or single bond;
A¹ represents N, A² represents C, A³ represents N, A⁴ represents CH or
A¹ represents CH, A² represents N, A³ represents C, A⁴ represents N;
and
Z represents aryl, heterocyclic ring A, C₁₋₄alkoxy-C₁₋₄alkyl, or where the valence allows, Z may optionally be a 3, 4, 5 or 6 membered spirocyclic ring C comprising only carbon ring atoms, as shown in formula II wherein y is 1, 2, 3 or 4, and * marks the points of ring fusion in formula I:

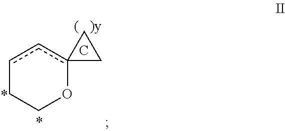

m represents 1 or 2;
n represents 1 or 2;
A⁵-R² represents N—R², N—C(H)R²R³, CR²R³ or CR³—CH₂—R², wherein only a C or a N atom are ring forming atoms, and A⁵ is N—, N—C(H)(R³)—, CR³— or C(R³)—CH₂—;
R³ represents hydrogen, C₁₋₇alkyl, hydroxy;
R² represents heterocyclyl ring B, said heterocyclyl ring B containing from 3 to 12 ring forming atoms (wherein said ring forming atoms may include bridging atoms), comprising 1, 2, 3 or 4 nitrogen atoms, and comprising 0, 1, 2 or 3 oxygen atoms, and comprising 0, 1, 2 or 3 sulfur atoms,
   being saturated, partly saturated, or unsaturated,
   being optionally substituted by one to four substituents, the substituents being independently selected from the group consisting of halo, cyano, oxo, hydroxy, amino, nitro, C₁₋₇alkyl, C₁₋₇alkoxy, hydroxy-C₁₋₇alkyl, aminocarbonyl, C₁₋₇alkylaminocarbonyl, di(C₁₋₇alkyl)aminocarbonyl, C₁₋₇alkoxycarbonyl, C₁₋₇alkylcarbonyl; or
      R² represents OH, SH, C₁₋₇alkoxy, C₁₋₇alkylthio, amino, C₁₋₇alkylcarbonylamino, C₃₋₇ cycloalkyl-carbonylamino, C₁₋₇alkylsulfonylamino;
      or when A⁵-R² represents CR²R³, R² and R³ may join, together with the carbon to which they are attached, to form a 5-membered spirocyclic group, said spirocyclic group comprising 3 carbon ring atoms and 2 ring heteroatoms independently selected from O and N, and wherein said spirocyclic group is substituted at one carbon ring atom with an oxo substituent.

Heterocyclic ring A is a saturated, partially saturated or unsaturated ring comprising 5, 6, 7, 8, 9 or 10, preferably 5 or 6 ring atoms, wherein one or more, preferably from one to four, especially one or two ring atoms are a heteroatom independently selected from N, O and S (the remaining ring atoms therefore being carbon), preferably N or O. Heterocyclic ring A is optionally substituted with one or two substituents selected from oxo (═O), thiono (═S), imino (═NH), imino-lower alkyl, halogen, amino, N-lower alkylamino, N,N-di-lower alkylamino, N-lower alkanoylamino, N,N-di-lower alkanoylamino, hydroxy, lower alkoxy, lower alkoxylower alkoxy, lower alkanoyl, lower alkanoyloxy, cyano, nitro, carboxy, lower alkoxycarbonyl, carbamoyl, amidino, guanidino, ureido, mercapto, and lower alkylthio. Preferably, heterocyclic ring A is unsubstituted. Examples of heterocyclic ring A include but are not limited to pyrrolidinyl, piperidyl, piperazinyl, morpholinyl, thiomorpholinyl, tetrahydrofuryl, tetrahydro-2H-pyranyl, pyranyl, pyridinyl, pyridazinyl and pyrimidinyl.

When Z is aryl, said aryl and optional substituents of aryl are as defined herein. Aryl is preferably naphthyl or phenyl, preferably phenyl. Preferably when Z is aryl, said aryl is unsubstituted.

The compounds of formula I therefore permit, for example, a therapeutic approach, especially for diseases in the treatment of which, and also for the prevention of which, an inhibition of the IGF-IR tyrosine kinase and/or of the IGF-IR-dependent cell proliferation shows beneficial effects. Such diseases include proliferative diseases, such as tumours, like for example breast, renal, prostate, colorectal, thyroid, ovarian, pancreas, neuronal, lung, uterine and gastro-intestinal tumours as well as osteosarcomas and melanomas. Compounds of the invention show improved efficacy, tolerability and/or selectivity when compared to known IGF-IR inhibitors. Without being bound to theory, it is believed that several factors contribute to the improvements in efficacy and tolerability, for example increased metabolic stability and the reduced formation of multiple kinase-active metabolites. Although known compounds have been shown to produce desirable effects in in-vivo models through the inhibition of IGF-1 receptor activity, they have been found to undergo extensive metabolism. This not only limits the pharmacokinetic profile of such derivatives, but also generates metabolites, which show multiple potent kinase activities. Advantageously, compounds of formula I also exhibit improved selectivities with respect to the inhibition of other kinases, including other tyrosine kinases.

The invention may be more fully appreciated by reference to the following description, including the following glossary of terms and the concluding examples. As used herein, the terms “including”, “containing” and “comprising” are used herein in their open, non-limiting sense. Where compounds of formula I are mentioned, this is meant to include also the tautomers and N-oxides of the compounds of formula I.

Where the plural form is used for compounds, salts, and the like, this is taken to mean also a single compound, salt, or the like.

Any formula given herein is intended to represent compounds having structures depicted by the structural formula as well as certain variations or forms. In particular, compounds of any formula given herein may have asymmetric centers and therefore exist in different enantiomeric or diastereoisomeric forms. If at least one asymmetrical carbon atom is present in a compound of the formula (I), such a compound may exist in optically active form or in the form of a mixture of optical isomers, e.g. in the form of a racemic mixture. All optical isomers and their mixtures, including the racemic mixtures, are part of the present invention. Thus, any given formula given herein is intended to represent a racemate, one or more enantiomeric forms, one or more diastereomeric forms, one or more atropisomeric forms, and mixtures thereof. Furthermore, certain structures may exist as geometric isomers (i.e. cis and trans isomers), as tautomers, or as atropisomers.

Any formula given herein is intended to represent hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as 2H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H, ¹³C, and ¹⁴C, are present. Such isotopically labelled compounds are useful in metabolic studies (preferably with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly preferred for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or 1D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this invention any atom specifically designated as a deuterium (D) is meant to represent deuterium, for example in the ranges given above.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula (where one or more up to all more general expressions in embodiments characterized as preferred above or below can be replaced with a more specific definition, thus leading to a more preferred embodiment of the invention, respectively).

The general terms used hereinbefore and hereinafter preferably have within the context of this disclosure the following meanings, unless otherwise indicated:

Benzene-fused 6-membered oxygen-containing heterocyclic refers to derivatives having a basic core of chroman, or chromene such as 2H-chromene or 4H-chromene (chrom-3-ene or chrom-2-ene).

Carbon containing groups, moieties or molecules contain 1 to 7, preferably 1 to 6, more preferably 1 to 4, most preferably 1 or 2, carbon atoms. Any non-cyclic carbon containing group or moiety with more than 1 carbon atom is straight-chain or branched. The prefix “lower” denotes a radical having 1 to 7, preferably 1 to 4 carbon atoms, the radicals in question being either unbranched or branched with single or multiple branching.

“Alkyl” refers to a straight-chain or branched-chain alkyl group, preferably represents a straight-chain or branched-chain C₁₋₁₂alkyl, for example, methyl, ethyl, n- or iso-propyl, n-, iso-, sec- or tert-butyl, n-pentyl, n-hexyl, n-heptyl, n-octyl, 2-ethylhexyl, n-nonyl, n-decyl, n-undecyl, n-dodecyl. A “lower alkyl” is, for example, methyl, ethyl, n-propyl, isopropyl, n-butyl, isobutyl, sec-butyl, tert-butyl, n-pentyl, isopentyl, neopentyl, n-hexyl or n-heptyl. Thus, C₁₋₇ alkyl are either unbranched or branched (with single or multiple branching) alkyl radicals having from 1 to 7 carbon atoms, respectively, and include methyl, ethyl, n-propyl, 2-propyl, n-butyl, sec-butyl, t-butyl, and the like.

Each alkyl part of other groups like “alkoxy”, “alkoxyalkyl”, “alkoxycarbonyl”, “alkoxy-carbonylalkyl”, “alkylsulfonyl”, “alkylsulfoxyl”, “alkylamino”, “haloalkyl” shall have the same meaning as described in the abovementioned definition of “alkyl”.

“Alkylene” refers to an alkyl-biradical. Consequently, “lower alkylene” is, for example, methylene (—CH₂—), ethylene (—CH₂—CH₂—), propylene (—CH₂—CH₂—CH₂—) or tetramethylene (—CH₂—CH₂—CH₂—CH₂—).

“Cycloalkyl” refers to a saturated or partially saturated, monocyclic, fused polycyclic, or Spiro polycyclic, carbocycle having from 3 to 12 ring atoms per carbocycle. Illustrative examples of cycloalkyl groups include the following moieties: cyclopropyl, cyclobutyl, cyclopentyl and cylclohexyl. The term cycloalkyl excludes “aryl”.

A “substituted alkyl” or “substituted lower alkyl” is alkyl/lower alkyl as defined above where one or more, preferably one to three, substituents may be present, such as halogen, amino, N-lower alkylamino, N,N-di-lower alkylamino, N-lower alkanoylamino, N,N-di-lower alkanoylamino, hydroxy, lower alkoxy, lower alkoxy-lower alkoxy, lower alkanoyl, lower alkanoyloxy, cyano, nitro, carboxy, lower alkoxycarbonyl, carbamoyl, amidino, guanidino, ureido, mercapto, lower alkylthio. Further, a substituted alkyl may be substituted by a heterocyclic radical as defined herein. Further, a substituted alkyl may be substituted by a cycloalkyl as defined herein; an example of such a case is the moiety —CH₂-cyclopropyl.

A “substituted cycloalkyl” is a cycloalkyl as defined above wherein one or more substituents, preferably one to three substituents, may be present, said substituens are as defined above for “substituted alkyl” and also include “alkyl” itself (e.g. methyl). Consequently, a moiety like —(CH₃)cyclopropyl is considered substituted cycloalkyl.

“Halogen” (or “halo”) denotes fluorine, bromine, chlorine or iodine, in particular fluorine, chlorine. Halogen-substituted groups and moieties, such as alkyl substituted by halogen (haloalkyl) can be mono-, poly- or per-halogenated.

“Halo-alkyl” refers to an alkyl as defined herein, that is substituted by one or more halo groups as defined herein. The halo-alkyl can be mono-halo-alkyl, di-halo-alkyl or poly-halo-alkyl including per-halo-alkyl. A mono-halo-alkyl can have one iodo, bromo, chloro or fluoro within the alkyl group. Di-halo-alkyl and poly-halo-alkyl groups can have two or more of the same halo atoms or a combination of different halo groups within the alkyl. Typically the poly-halo-alkyl contains up to 12, or 10, or 8, or 6, or 4, or 3, or 2 halo groups. Non-limiting examples of halo-alkyl include fluoro-methyl, di-fluoro-methyl, tri-fluoro-methyl, chloro-methyl, di-chloro-methyl, tri-chloro-methyl, penta-fluoro-ethyl, hepta-fluoro-propyl, di-fluoro-chloro-methyl, di-chloro-fluoro-methyl, di-fluoro-ethyl, di-fluoro-propyl, di-chloro-ethyl and dichloro-propyl. A per-halo-alkyl refers to an alkyl having all hydrogen atoms replaced with halo atoms.

Hetero atoms are atoms other than Carbon and Hydrogen, preferably nitrogen (N), oxygen (O) or sulfur (S), in particular nitrogen or oxygen.

“Heterocyclyl” or “heterocycle” refers to a heterocyclic radical that is saturated, partially saturated or unsaturated and is preferably a monocyclic or a polycyclic ring (in case of a polycyclic ring particularly a bicyclic, tricyclic or spirocyclic ring); and has 3 to 24, more preferably 4 to 16, most preferably 5 to 10 and most preferably 5 or 6 ring atoms; wherein one or more, preferably one to four, especially one or two ring atoms are a heteroatom (the remaining ring atoms therefore being carbon). The bonding ring (i.e. the ring connecting to the molecule) preferably has 4 to 12, especially 5 to 7 ring atoms. The term heterocyclyl includes heteroaryl. The heterocyclic radical (heterocyclyl) may be unsubstituted or substituted by one or more, especially 1 to 3, substituents independently selected from the group consisting of the substituents defined above for substituted alkyl and I or from one or more of the following substituents: alkyl, oxo (═O), thiono (═S), imino (═NH), imino-lower alkyl.

A polycyclic heterocyclic moiety may be annellated to a further saturated, partly saturated or unsaturated ring, forming a polycyclic heterocyclic radical. Such polycyclic heterocyclic radical includes moieties wherein one or two benzene radicals are annellated to a moncyclic heterocyclic radical as defined above to form a chromane-radical. Further, a polycyclic heterocyclic moiety may be bridged by an alkandiyl or alkendiyl as defined herein. Further, a polycyclic heterocyclic moiety may be connected to a further heterocyclyl or cycloalkyl via one connecting atom to form a spirocyclic heterocyclic moiety. In the context of R², preferred heterocyclic radicals are saturated heterocyclic radicals and contain at least one nitrogen ring atom whereby the binding of the heterocyclic radical to the radical of the molecule of formula I occurs preferably via a nitrogen ring atom. Most preferably a heterocyclic radical is azetidinyl, pyrrolidinyl, piperidyl, azepanyl, piperazinyl, morpholinyl or thiomorpholinyl, wherein said radicals are optionally substituted by one or more, preferably one or two, radicals selected independently of one another from the group consisting of oxo, lower alkyl, hydroxy-lower alkyl, hydroxy, lower alkoxycarbonyl, carbamoyl, phenyl and pyridyl. In the context of Z, preferred heterocycles are saturated heterocycles and contain at least one oxygen ring atom whereby the binding of the heterocyclic radical to the radical of the molecule of formula I occurs preferably via a carbon ring atom. Most preferably a heterocycle is selected from the group consisting of oxetane, tetrahydrofuryl, tetrahydro-2H-pyranyl, pyranyl, 2-oxabicyclo[1.1.1]pentanyl; 5-oxabicyclo[2.1.1]hexanyl; 2-oxabicyclo[2.1.1]hexanyl, 6-oxabicyclo[3.1.1]heptanyl; 2-oxabicyclo[2.2.1]heptanyl; 2-oxabicyclo[3.1.1]heptanyl, 7-oxabicyclo[2.2.1]heptanyl; 8-oxabicyclo[3.2.1]octanyl; 2-oxabicyclo[2.2.2]octanyl; 6-oxabicyclo[3.2.1]octanyl and 2-oxabicyclo[3.2.1]octanyl, wherein said heterocycles are optionally substituted by one or more, preferably one or two, radicals selected independently of one another from the group consisting of lower alkyl, hydroxy-lower alkyl, hydroxy, lower alkoxycarbonyl, carbamoyl, phenyl and pyridyl.

“Aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together. Preferably, the aryl is a C₆-C₁₀-aryl. Non-limiting examples include phenyl or naphthyl each of which may be unsubstituted or substituted by 1-4 substituents, preferably by 1-2 substituents, selected from the group consisting of C₁-C₇-alkyl, halo-C₁-C₇-alkyl, halogen, hydroxy, C₁-C₇-alkoxy, amino, nitro or cyano. As used herein, the term “aryl” preferably refers to unsubstituted phenyl or substituted phenyl, wherein the substituents for substituted phenyl are those as described above for “aryl”. As used herein, the term “aryl” most preferably refers to unsubstituted phenyl.

“Treatment” includes prophylactic (preventive) and therapeutic treatment as well as the delay of progression of a disease or disorder.

“Salts” (which, what is meant by “or salts thereof” or “or a salt thereof”), can be present alone or in mixture with free compound of the formula (I) and are preferably pharmaceutically acceptable salts.

Such salts are formed, for example, as acid addition salts, preferably with organic or inorganic acids, from compounds of formula (I) with a basic nitrogen atom, especially the pharmaceutically acceptable salts. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient. The salts of compounds of formula (I) are preferably pharmaceutically acceptable salts; suitable counterions forming pharmaceutically acceptable salts are known in the field.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

“Combination” refers to either a fixed combination in one dosage unit form, or a kit of parts for the combined administration where a compound of the formula (I) and a combination partner (e.g. an other drug as explained below, also referred to as “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula (I) and a combination partner, are both administered to a patient simultaneously in the form of a single entity or dosage. The term “non-fixed combination” means that the active ingredients, e.g. a compound of formula (I) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

In preferred embodiments, which are preferred independently, collectively or in any combination or sub-combination, the invention relates to a compound of the formula I, in free base form or in acid addition salt form, wherein the substituents are as defined herein.

The invention further relates to pharmaceutically acceptable prodrugs of a compound of formula (I). Particularly, the present invention also relates to pro-drugs of a compound of formula I as defined herein that convert in vivo to the compound of formula I as such. Any reference to a compound of formula I is therefore to be understood as referring also to the corresponding pro-drugs of the compound of formula I, as appropriate and expedient.

The invention further relates to pharmaceutically acceptable metabolites of a compound of formula (I).

In another embodiment, the invention relates to a compound of formula I, or a salt thereof, wherein
A¹ represents N, A² represents C, A³ represents N, A⁴ represents CH; or
A¹ represents CH, A² represents N, A³ represents C, A⁴ represents N; and
Z represents optionally substituted aryl, optionally substituted heterocycle;
m represents 1 or 2;
n represents 1 or 2;
A⁵-R² represents N—R², NC(H)R²R³, CR²R³ or CR³—CH₂—R²;
R³ represents hydrogen, C₁₋₇alkyl, hydroxy:
R² represents heterocyclyl, said heterocyclyl
   containing 3-12 ring forming atoms,
   containing 1-3 nitrogen atoms, 0-3 oxygen atoms, 0-3 sulfur atoms,
   being saturated or partly saturated,
   being optionally substituted by one to four substituents, the substituents being independently selected from the group consisting of halo, cyano, oxo, hydroxy, amino, nitro, C₁₋₇alkyl, C₁₋₇alkoxy, hydroxy-C₁₋₇alkyl, aminocarbonyl, C₁₋₇alkylaminocarbonyl, di(C₁₋₇alkyl)aminocarbonyl, C₁₋₇alkoxycarbonyl; or
      R² represents OH, SH, C₁₋₇alkoxy, C₁₋₇alkylthio, amino, C₁₋₇alkylcarbonylamino, C₁₋₇alkylsulfonylamino.

In one embodiment, the present invention provides a compound of formula I, depicted by formula I-1

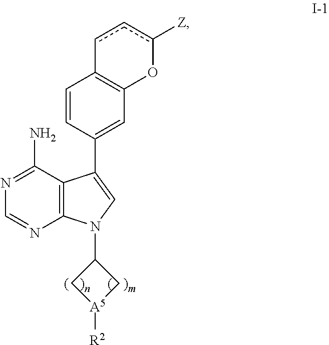

wherein the substituents are as defined herein. Compounds of formula I-1 may be considered as 5,7-disubstituted derivatives of 7H-pyrrolo[2,3-d]pyrimidin-4-amine.

In a further advantageous embodiment, the present invention provides a compound of formula I, depicted by formula I-2

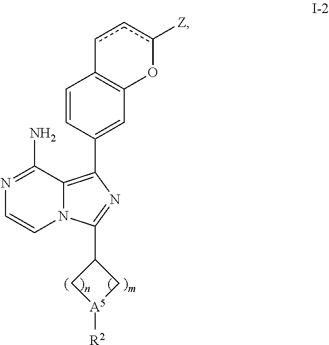

wherein the substituents are as defined herein. Compounds of formula I-2 may be considered as 1,3-disubstituted derivatives of imidazo[1,5-a]pyrazin-8-amine.

In a further advantageous embodiment, the present invention provides a compound of formula I, depicted by formula I-a

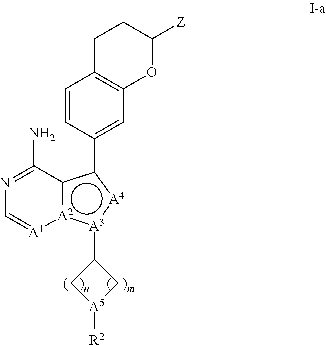

wherein the substituents are as defined herein.

In a further advantageous embodiment, the present invention provides a compound of formula I, depicted by formula I-b

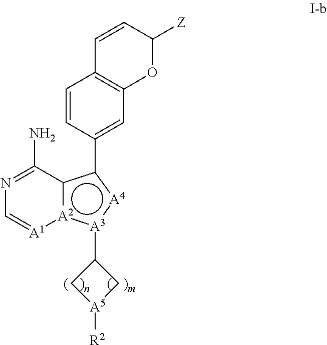

wherein the substituents are as defined herein.

In a further advantageous embodiment, the present invention provides a compound of formula I, depicted by formula I-c

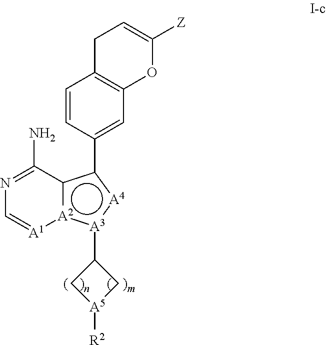

wherein the substituents are as defined herein.

A “compound of formula I” as mentioned herein, includes a compound of formula I-1, I-2, I-a, I-b and I-c above. Embodiments of the invention as described herein, including those relating to a compound of formula I, can also relate to each of the compounds of formula I-1, I-2, I-a, I-b and I-c separately.

In a preferred embodiment, there is provided a compound of formula I-1.

In another preferred embodiment, [symbol] represents a single bond.

In a preferred embodiment there is provided a compound of formula (I) wherein the stereochemistry is as shown below in figure i:

i:

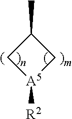

In an alternative embodiment the stereochemistry is as shown below in Figure ii:

ii:

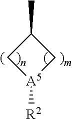

In another embodiment, Z represents optionally substituted aryl, or optionally substituted heterocycle;

In a further embodiment, Z represents:
unsubstituted phenyl,
heterocyclic ring A, wherein said heterocyclic ring A is an unsubstituted 5 or 6 membered saturated, partially saturated or unsaturated ring comprising 1 or 2 ring hetero atoms independently selected from N, O and S, preferably N and O.
—C₁₋₄alkoxy-C₁₋₄alkyl,
or a 6 membered spirocyclic ring C comprising only carbon ring atoms.

In a preferred embodiment, Z represents phenyl, tetrahydrofuranyl, C₁₋₄alkoxy-C₁₋₄alkyl, pyridinyl or a 6 membered spirocyclic ring C comprising only carbon ring atoms.

In another preferred embodiment, Z represents phenyl or tetrahydrofuranyl, preferably tetrahydrofuran-2-yl.

In a further embodiment, the present invention provides a compound of formula I wherein the substituents are as defined herein, and Z represents optionally substituted phenyl.

In a further embodiment, the present invention provides a compound of formula I wherein the substituents are as defined herein, and Z represents unsubstituted phenyl.

In a further embodiment, m represents 1. In another embodiment, n represents 1.

In a further embodiment, both m and n represent 1. In an alternative embodiment, both m and n represent 2.

In another embodiment, R³ represents H, C₁₋₄ alkyl (preferably methyl), or hydroxy, preferably H or OH.

In a further embodiment, A⁵-R² represents CR²R³ or CR³—CH₂—R². In a more particular embodiment, when A⁵-R² represents CR³—CH₂—R², R³ is H or OH.

In another embodiment, A⁵-R² represents CR²R³ or CR³—CH₂—R² where R³ represents C₁₋₇alkyl or hydroxy, preferably methyl or hydroxy.

In a further embodiment, A⁵-R² represents CHR² or CH—CH₂—R².

In a further embodiment, A⁵ represents C(CH₃)—, C(OH)—CH₂—, CH— or CH—CH₂— (wherein only one C atom is a ring-forming atom), preferably A⁵ represents CH— or CH—CH₂—. A⁵ may form, together with the carbon atoms in the ring of which A⁵ is a ring member, a cyclobutane-, cyclobutanemethylene-, cyclopentane-, cyclopentanemethylene-, cyclohexane-, cyclohexanemethylene-moiety. In a preferred embodiment, A⁵ may form, together with the carbon atoms in the ring of which A⁵ is a ring member, a cyclobutane-, cyclobutanemethylene-, cyclohexane-, or cyclohexanemethylene-moiety.

In an alternative embodiment A⁵-R² represents CR²R³, wherein, when m and n are both 1, R² and R³ join, together with the carbon to which they are attached, to form a 5-membered spirocyclic group, said spirocyclic group selected from the spirocyclic rings shown below:

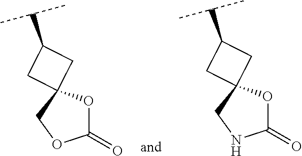

In another embodiment, R² represents heterocyclyl ring B, wherein said heterocyclyl ring B
contains 3-12 ring forming atoms,
contains 1-3 nitrogen atoms, 0-3 oxygen atoms, 0-3 sulfur atoms,
is saturated or partly saturated,
and is optionally substituted by one to four substituents, the substituents being independently selected from the group consisting of halo, cyano, oxo, hydroxy, amino, nitro, C₁₋₇alkyl, C₁₋₇alkoxy, hydroxy-C₁₋₇alkyl, aminocarbonyl, C₁₋₇alkylaminocarbonyl, di(C₁₋₇alkyl)aminocarbonyl, C₁₋₇alkoxycarbonyl; or
   R² represents OH, SH, C₁₋₇alkoxy, C₁₋₇alkylthio, amino, C₁₋₇alkylcarbonylamino, C₁₋₇alkylsulfonylamino.

In a further embodiment, R² represents heterocyclyl ring B, said heterocyclyl ring B: containing from 5-6 ring forming atoms; containing from 1-2 nitrogen atoms, from 0-1 oxygen atoms, from 0-1 sulfur atoms; being saturated; and being optionally substituted by one or two substituents, the substituents being independently selected from the group consisting of oxo, hydroxy, methyl, hydroxymethyl, ethyl, aminocarbonyl, and ethoxycarbonyl.

In a further embodiment, R² represents OH, C₁₋₇alkoxy, SH, C₁₋₇alkylthio, amino, C₁₋₇alkylcarbonylamino, C₁₋₇alkylsulfonylamino.

In a further embodiment, R² represents heterocyclyl ring B as defined herein, said heterocyclyl ring B being bound to A⁵ via a ring nitrogen atom.

In a further embodiment, R² represents a heterocyclyl ring B selected from the following heterocyclic moieties:

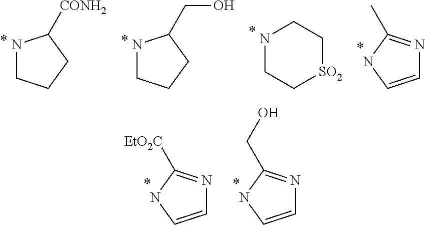

wherein the marked atom is bound to A⁵.

In a further embodiment, R² represents hydroxy, amino, methylcarbonylamino, methylsulfonylamino methoxy, ethoxy, propoxy, iso-propoxy, thio, methylthio, ethylthio, propylthio, iso-proylthio, particularly methylthio or hydroxy.

In another embodiment, R² is selected from OH, SH, C₁₋₄alkoxy, C₁₋₄alkylthio, amino, C₁₋₄alkylcarbonylamino, C₃₋₆cycloalkyl-carbonylamino and C₁₋₄alkylsulfonylamino.

In another embodiment, R² is heterocyclyl ring B, which is a 5 or 6 membered saturated, partly saturated, or unsaturated ring, wherein the 6-membered saturated ring optionally contains a —CH₂— or —CH₂—CH₂— bridge, and wherein the ring comprises at least 1 N ring atom and
optionally 1, 2 or 3 additional N atoms, or
optionally an additional S atom, said S atom being optionally substituted by 1 or 2 ═O substituents,
   and wherein heterocyclyl ring B is optionally substituted at a C and/or N atom or atoms with one or two substituents selected from oxo, hydroxy, amino, C₁₋₄alkyl, C₁₋₄alkoxy, hydroxy-C₁₋₄alkyl, aminocarbonyl, C₁₋₄alkylaminocarbonyl, di(C₁₋₄alkyl)aminocarbonyl, C₁₋₄alkoxycarbonyl and C₁₋₄alkylcarbonyl.

In another embodiment, R² is selected from OH, NH₂, —NHCOCH₃, —NHSO₂CH₃, —NH—CO-cyclopropyl, —NH—CO-isopropyl, and —NH—CO-methyl, or R² is selected from any one of the following groups, where * indicates the point of attachment:

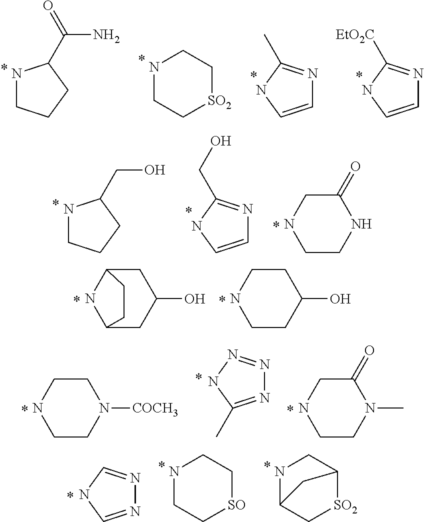

In a particularly advantageous embodiment, the present invention relates to a compound of formula I mentioned in the Examples below, or a salt, especially a pharmaceutically acceptable salt, thereof.

In a particularly preferred embodiment, the group attached to A3 is selected from:

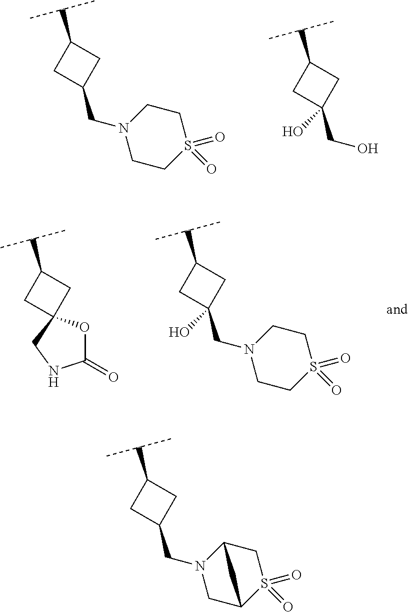

In another embodiment, there is provided a compound of formula I wherein:
the single/double bond, A¹, A², A³ and A⁴ are as described for formula I;
Z represents phenyl, tetrahydrofuranyl, C₁₋₄alkoxy-C₁₋₄alkyl, pyridinyl or a 6 membered spirocyclic ring C comprising only carbon ring atoms;
both m and n represent 1, or both m and n represent 2;
A⁵-R² represents CR²R³ or CR³—CH₂—R²
R² is selected from OH, NH₂, —NHCOCH₃, —NHSO₂CH₃, —NH—CO-cyclopropyl, —NH—CO-isopropyl, and —NH—CO-methyl, or R² is selected from any one of the following groups, where * indicates the point of attachment:

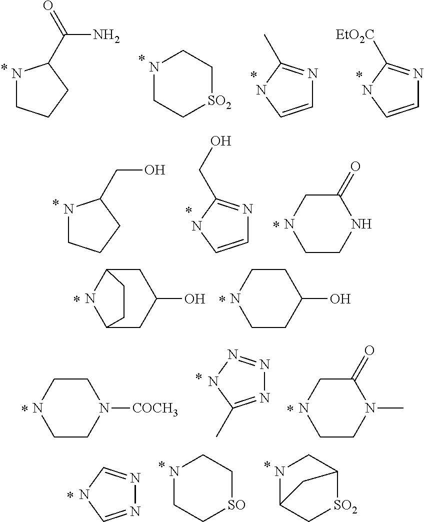

or A⁵-R² represents CR²R³, wherein, when m and n are both 1, R² and R³ join, together with the carbon to which they are attached, to form a 5-membered spirocyclic group, said spirocyclic group selected from the spirocyclic rings shown below:

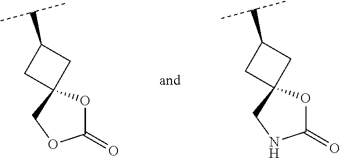

and
R³ represents hydrogen, C₁₋₇alkyl, hydroxy.

The invention relates in a second aspect to the manufacture of a compound of formula I. The compounds of formula I or salts thereof are prepared in accordance with processes known per se (see references cited above), though not previously described for the manufacture of the compounds of the formula I.

### General Reaction Processes:

In one embodiment, the invention relates to a process for manufacturing a compound of formula I (Method B) comprising the step of reacting a compound of formula IV

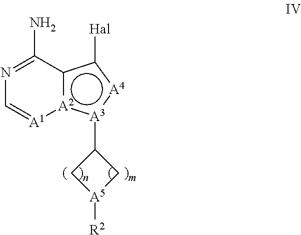

wherein the substituents are as defined above and Hal represents halogen, particularly iodo or bromo, with a compound of formula V,

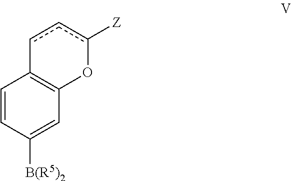

wherein the substituents are as defined above, and B(R⁵)₂ represents a cyclic or acyclic boronic acid, such as 4,4,5,5-tetramethyl-1,3,2-diocoborolane, in the presence of a catalyst, such as a Pd(0) catalyst, e.g. PO(PPh₃)₄, optionally in the presence of one or more reaction aids, such as a base, e.g. Na₂CO₃, optionally in the presence of one or more diluents, particularly polar solvents, e.g. H₂O/dmf. This type of reaction is also known as Suzuki reaction, typical reaction conditions are known in the field and may applied to the present process.

In a further embodiment, the invention relates to a process for manufacturing a compound of formula I (Method C) comprising the step of reacting a compound of formula VI

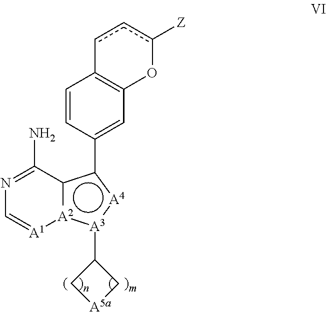

wherein the substituents are as defined above and A^{5a} represents CR³CHO, particularly CHCHO, with a compound of formula VII,

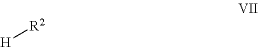

wherein R² are as defined above,
optionally in the presence of one or more reaction aids, such as a borohydride, e.g. triacetoxyborohydride, optionally in the presence of one or more diluents, particularly apolar solvents, e.g. dichloroethane. This type of reaction is also known as a reductive amination reaction, typical reaction conditions are known in the field and may applied to the present process. In this embodiment, the starting material, aldehyde VI may be formed in situ by oxidation of the corresponding alcohol, e.g. by using a hypervalent iodine reagent such as 2-iodoxybenzoic acid (IBX).

In a further embodiment, the invention relates to a process for manufacturing a compound of formula I (Method D) comprising the step of reacting a compound of formula IIX

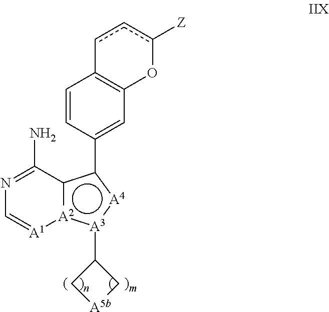

wherein the substituents are as defined above and A^{5b} represents CR³CH2O—FG (FG is a hydroxy activating group), particularly CHCH₂OTs (Ts represents tosylate), with a compound of formula IX,

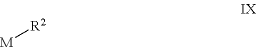

wherein R² is as defined above, particularly thio or alkylthio, and M represents an (earth) alkali metal, particularly sodium,
optionally in the presence of one or more reaction aids, optionally in the presence of one or more diluents, particularly polar solvents, e.g. THF. Typical reaction conditions are known in the field and may applied to the present process.

In a further embodiment, the invention relates to a process for manufacturing a compound of formula I (Method E) comprising the step of reacting a compound of formula X

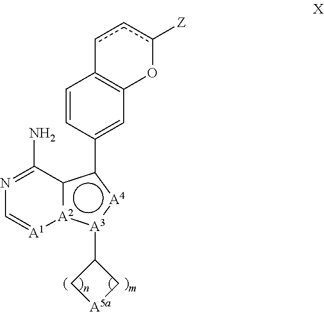

wherein the substituents are as defined above and A^{5b} represents an azide containing group, eg CR³CH2N3, with a reducing agent, eg the Staudinger reduction with triphenyl phosphine and water. This type of reaction is known as an azide reduction and the product is the corresponding primary amine.

In a further embodiment, the invention relates to a process for manufacturing a compound of formula I (Method F) comprising the step of reacting a compound of formula XI

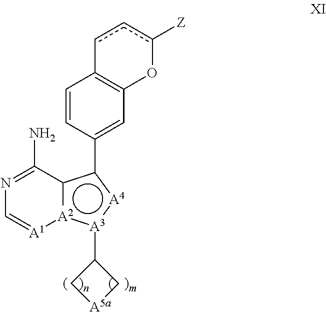

wherein the substituents are as defined above and A^{5b} represents an amino containing group, eg CR³CH2NH2, with an acylating or a sulphonylating agent, eg acetyl chloride or methane sulphonyl chloride. This type of reaction is known as an acylation or sulphonylation reaction and the product is the corresponding amide or sulphonamide.

In a further embodiment, the invention relates to a process for manufacturing a compound of formula I (Method G) comprising the step of reacting a compound of formula XII

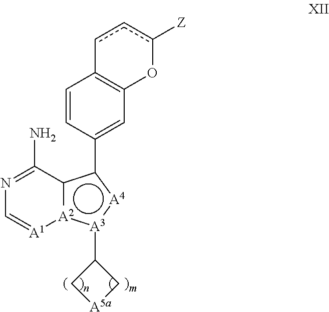

wherein the substituents are as defined above and A^{5b} represents an ester containing group with a reducing agent, eg lithium aluminium chloride. This type of reaction is known as an ester reduction and the product is the corresponding primary alcohol.

In a further embodiment, the invention relates to a process for manufacturing a compound of formula I (Method G) comprising the step of reacting a compound of formula XIII

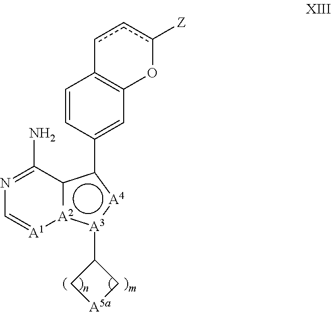

wherein the substituents are as defined above and A^{5b} represents a ketone containing group, eg CO, with a reducing agent, eg L-selectride. This type of reaction is known as a ketone reduction and the product is the corresponding alcohol.

In a further embodiment, the invention relates to a process for manufacturing a compound of formula I (Method H) comprising the step of reacting a compound of formula XIIV

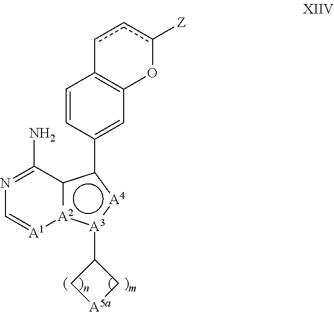

wherein the substituents are as defined above and A^{5b} represents a diol or amino alcohol containing group, with phosgene or an equivalent, eg dicarbonyl imidazole. This type of reaction is known as a cyclisation reaction and the product is the corresponding cyclic carbonate or cyclic carbamate, eg oxazolidinone.

In a further embodiment, the invention relates to a process for manufacturing a compound of formula I (Method I) comprising the step of reacting a compound of formula XIV

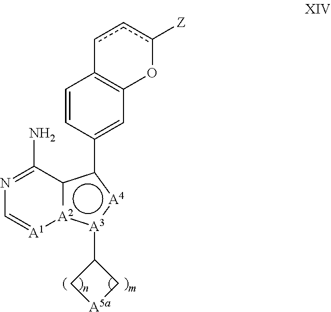

wherein the substituents are as defined above and A^{5b} represents an alcohol containing group, eg CH(OH) with benzoic acid, triphenylphosphine and diisopropyl azodicarboxylate. This type of reaction is known as a Mitsunobu reaction and the product is the inverted alcohol.

### Protecting Groups:

In the methods describe above, functional groups which are present in the starting materials and are not intended to take part in the reaction, are present in protected form if necessary, and protecting groups that are present are cleaved, whereby said starting compounds may also exist in the form of salts provided that a salt-forming group is present and a reaction in salt form is possible. In additional process steps, carried out as desired, functional groups of the starting compounds which should not take part in the reaction may be present in unprotected form or may be protected for example by one or more protecting groups. The protecting groups are then wholly or partly removed according to one of the known methods. Protecting groups, and the manner in which they are introduced and removed are described, for example, in “Protective Groups in Organic Chemistry”, Plenum Press, London, New York 1973, and in “Methoden der organischen Chemie”, Houben-Weyl, 4th edition, Vol. 15/1, Georg-Thieme-Verlag, Stuttgart 1974 and in Theodora W. Greene, “Protective Groups in Organic Synthesis”, John Wiley & Sons, New York 1981. A characteristic of protecting groups is that they can be removed readily, i.e. without the occurrence of undesired secondary reactions, for example by solvolysis, reduction, photolysis or alternatively under physiological conditions.

### Additional Process Steps:

In the methods described above, a compound of formula I thus obtained may be converted into another compound of formula I, a free compound of formula I is converted into a salt, an obtained salt of a compound of formula I is converted into the free compound or another salt, and/or a mixture of isomeric compounds of formula I is separated into the individual isomers. The end products of formula I may however also contain substituents that can also be used as protecting groups in starting materials for the preparation of other end products of formula I. Thus, within the scope of this text, only a readily removable group that is not a constituent of the particular desired end product of formula I is designated a “protecting group”, unless the context indicates otherwise.

A compound of formula I can be converted to a corresponding N-oxide. The reaction is carried out with a suitable oxidizing agent, preferably a peroxide, for example m-chloroperbenzoic acid, in a suitable solvent, e.g. halogenated hydrocarbon, typically chloroform or dichloromethane, or in a lower alkanecarboxylic acid, typically acetic acid, preferably at a temperature between 0° C. and the boiling temperature of the reaction mixture, especially at about RT.

### General Process Conditions:

All process steps described here can be carried out under known reaction conditions, preferably under those specifically mentioned, in the absence of or usually in the presence of solvents or diluents, preferably those that are inert to the reagents used and able to dissolve them, in the absence or presence of catalysts, condensing agents or neutralising agents, for example ion exchangers, typically cation exchangers, for example in the H⁺ form, depending on the type of reaction and/or reactants at reduced, normal, or elevated temperature, for example in the range from −100° C. to about 190° C., preferably from about −80° C. to about 150° C., for example at −80 to −60° C., at RT, at −20 to 40° C. or at the boiling point of the solvent used, under atmospheric pressure or in a closed vessel, if need be under pressure, and/or in an inert, for example an argon or nitrogen, atmosphere.

The invention relates also to those embodiments of the process in which one starts from a compound obtainable at any stage as an intermediate and carries out the missing steps, or breaks off the process at any stage, or forms a starting material under the reaction conditions, or uses said starting material in the form of a reactive derivative or salt, or produces a compound obtainable by means of the process according to the invention under those process conditions, and further processes the said compound in situ. In the preferred embodiment, one starts from those starting materials which lead to the compounds described hereinabove as preferred.

The compounds of formula I (or N-oxides thereof), including their salts, are also obtainable in the form of hydrates, or their crystals can include for example the solvent used for crystallisation (present as solvates).

In the preferred embodiment, a compound of formula I is prepared according to the processes and process steps defined in the Examples.

### Starting Materials

New starting materials and/or intermediates, as well as processes for the preparation thereof, are likewise the subject of this invention. In the preferred embodiment, such starting materials are used and reaction conditions so selected as to enable the preferred compounds to be obtained.

The starting materials used in the above described processes are known, capable of being prepared according to known processes (see references cited above), or commercially obtainable; in particular, they can be prepared using processes as described in the Examples.

In the preparation of starting materials, existing functional groups which do not participate in the reaction should, if necessary, be protected. Preferred protecting groups, their introduction and their removal are described above or in the examples. In place of the respective starting materials and transients, salts thereof may also be used for the reaction, provided that salt-forming groups are present and the reaction with a salt is also possible. Where the term starting materials is used hereinbefore and hereinafter, the salts thereof are always included, insofar as reasonable and possible.

The invention relates in a third aspect to the use of compounds of the present invention as pharmaceuticals. Particularly, the compounds of formula I have valuable pharmacological properties, as described hereinbefore and hereinafter. The invention thus provides:

a compound of the formula (I) as defined herein, as pharmaceutical/for use as pharmaceut-ical;
a compound of the formula (I) as defined herein, as medicament/for use as medicament;
a compound of the formula (I) as defined herein, for the treatment of/for use in the treatment of one or more IGF-1R mediated disorders or diseases;
the use of a compound of formula (I) as defined herein, for the manufacture of a medicament for the treatment of an IGF-1R mediated disorder or disease;
the use of a compound of formula (I) as defined herein, for the treatment of an IGF-1R mediated disorder or disease;
the use of a compound of formula I as defined herein for the inhibition of the IGF-IR tyrosine kinase;
the use of a compound of formula (I) as defined herein, for the treatment of a disorder or disease selected from multiple myeloma, neuroblastoma, synovial, hepatocellular, Ewing's Sarcoma, adrenocotical carcinoma (ACC) or a solid tumor selected from osteosarcoma, melanoma, tumor of breast, renal, prostate, colorectal, thyroid, ovarian, pancreatic, lung, uterine or gastrointestinal tumor;
the use of a compound of formula (I) as defined herein, for the treatment of a disorder or disease selected from acute lung injury, pulmonary fibrosis and diabetic retinopathy;
a method of modulating IGF-1R activity in a subject, comprising the step of administering to a subject a therapeutically effective amount of a compound of formula I as definded herein;
a method for the treatment of an IGF-1R mediated disorder or disease comprising the step of administering to a subject a therapeutically effective amount of a compound of formula (I) as defined herein;
a method for inhibition IGF-1R in a cell, comprising contacting said cell with an effective amount of a compound of formula I as defined herein.

A “Subject in need thereof” refers to any animal classified as a mammal, including humans, domestic and farm animals, and zoo, sports, or pet animals, such as dogs, cats, cattle, horses, sheep, pigs, goats, rabbits, etc. In particular examples, the mammal is human.

The term “administration” or “administering” of the subject compound means providing a compound of the invention and prodrugs thereof to a subject in need of treatment. Administration “in combination with” one or more further therapeutic agents includes simultaneous (concurrent) and consecutive administration in any order, and in any route of administration. An “effective amount” of a compound is an amount sufficient to carry out a specifically stated purpose. An “effective amount” may be determined empirically and in a routine manner, in relation to the stated purpose.

The term “therapeutically effective amount” refers to an amount of a compound (e.g., an IGF-1R antagonist) effective to “treat” an IGF-1R-mediated disorder in a subject or mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition herein of “treating”. To the extent the drug may prevent growth and/or kill existing cancer cells, it may be cytostatic and/or cytotoxic.

The term “cancer” refers to the physiological condition in mammals that is typically characterized by unregulated cell growth/proliferation. Examples of cancer include, but are not limited to: carcinoma, lymphoma, blastoma, and leukemia. More particular examples of cancers include, but are not limited to: chronic lymphocytic leukemia (CLL), lung, including non small cell (NSCLC), breast, ovarian, cervical, endometrial, prostate, colorectal, intestinal carcinoid, bladder, gastric, pancreatic, hepatic (hepatocellular), hepatoblastoma, esophageal, pulmonary adenocarcinoma, mesothelioma, synovial sarcoma, osteosarcoma, head and neck squamous cell carcinoma, juvenile nasopharyngeal angiofibromas, liposarcoma, thyroid, me-lanoma, basal cell carcinoma (BCC), adrenocotical carcinoma (ACC), medulloblastoma and desmoid.

The term “IGF-1R mediated disease” includes but is not limited to, multiple myeloma, neu-roblastoma, synovial, hepatocellular, Ewing's Sarcoma, adrenocotical carcinoma (ACC), or a solid tumor selected from osteosarcoma, melanoma, tumor of breast, renal, prostate, colo-rectal, thyroid, ovarian, pancreatic, lung, uterine or gastrointestinal tumor.

It was further found that compounds of formula I are also useful in the treatment of acute lung injury and pulmonary fibrosis and diabetic retinopathy.

“Treating” or “treatment” or “alleviation” refers to both therapeutic treatment and prophylactic or preventative measures, wherein the object is to prevent or slow down (lessen) the targeted pathologic disease or condition or disorder. Those in need of treatment include those already with the disorder as well as those prone to having the disorder or those in whom the disorder is to be prevented (prophylaxis). When the IGF-1R-mediated disorder is cancer, a subject or mammal is successfully “treated” or shows a reduced tumor burden if, after receiving a therapeutic amount of an IGF-1R antagonist according to the methods of the present invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the IGF-1R antagonist may prevent growth and/or kill existing cancer cells, it may be cytostatic and/or cytotoxic. Reduction of these signs or symptoms may also be felt by the patient.

The invention provides in further embodiments methods to treat, ameliorate or prevent a condition which responds to inhibition of IGF-1R in a mammal suffering from said condition, comprising administering to the mammal a therapeutically effective amount of a compound of formula I as defined herein, and optionally in combination with a second therapeutic agent.

The compounds of the invention may be administered, for example, to a mammal suffering from an autoimmune disease, a transplantation disease, an infectious disease or a cell proliferative disorder. In particular examples, the compounds of the invention may be used alone or in combination with a chemotherapeutic agent to treat a cell proliferative disorder.

The efficacy of the compounds of the invention (i.e. a compound of formula I as defined herein) as inhibitors of IGF-IR tyrosine kinase activity can be demonstrated using a cellular “Capture ELISA”. In this assay the activity of the compounds of the invention against Insulin-like growth factor I (IGF-1) induced autophosphorylation of the IGF-IR is determined. The assay is conducted as follows: Compound-mediated inhibition of IGF1R and INSR phosphorylation in Hek293 cells transduced with the corresponding receptors is assessed in a capture ELISA format using the MSD (Meso Scale Discovery) platform. Briefly, 30′000 cells washed and diluted in starvation medium (DMEM high glucose supplemented with 0.1% BSA) are seeded in 90 μL per well into 96-well plates pre-coated with poly-D-lysine (0.1 mg/mL in PBS/0). After 24 h incubation at 37° C. and 5% CO2, dose-response effects are determined with 3-fold serial compound dilutions, starting at 10 μM. The final vehicle concentration is 0.1% DMSO in all wells. Following pre-incubation with compounds for 1 h, receptor phosphorylation is triggered by a 10 min exposure to 1.0 ng/μL IGF for Hek293-IGF1R cells, and 5.0 ng/μL insulin for Hek293-InsR cells. Cell lysis is achieved by addition of 80 μL MSD lysis buffer per aspirated well, incubation on ice for 20 min, and a freeze-thaw cycle. Target phosphorylation is then assessed by transferring volumes corresponding to approx. 6 μg Hek293-IGF1R or 0.6 μg Hek293-InsR lysates to MSD assay plates pre-coated with total-IGF1R or total-InsR Abs, respectively. After incubation for 2 h at rt, wells are exposed for 1 hr to a rabbit monoclonal antibody (CST #3024, 1:1000) detecting pIGF1R(Tyr1135/1136) as well as pINSR(Tyr1150/1151). Immune complexes are detected by a SULFO-Tag™-coupled anti-rabbit IgG antibody in the presence of 150 μL MSD read-buffer. Light emission at 620 nm triggered by application of electric current is recorded on a MSD SectorImager 6000. Acquired raw data (mean Ru-ECL units) are processed in an Excel analysis template. The plate blank (MSD lysis buffer) is subtracted from all data points. The effect of a particular test compound concentration on receptor phosphorylation is expressed relative to the window defined by ligand-stimulated vs unstimulated control cells (set as 100%). IC50 values [nM] are determined using 4-parametric curve-fitting (XLfit software, V4.3.2). Alternatively, the assay can be conducted with a slightly different format; Compound-mediated inhibition of IGF-1R and InsR phosphorylation in HEK293 cells overexpressing the corresponding receptors were assessed by quantitative Western blot using an Odyssey infrared imager as readout.

Briefly, 1,200,000 cells washed and diluted in starvation medium (DMEM high glucose supplemented with 0.1% BSA) are seeded in 2 mL per well into 6-well plates. After 6-8 hrs incubation at 37° C. and 5% CO2, dose-response effects are determined with 3-fold serial dilutions. The final vehicle concentration is=<0.1% DMSO in all wells. Following pre-incubation with compounds for 1 hr, receptor phosphorylation is triggered by a 10-min exposure to 500 ng/mL IGF1 for HEK293-IGF1R cells, and 5 μg/mL insulin for HEK293-INSR cells. Whole cell extracts are prepared by addition of 200 μL ice-cold lysis buffer for 10 min and a freeze-thaw cycle of 30 min, and 20 μg are loaded onto 48-well 8% acrylamide E-PAGE gels, then proteins are separated by electrophoresis for 36 min and transferred onto PVDF membranes using the iBlot transfer system for 7 min. Target phosphorylation is then assessed by incubating the membranes with a rabbit mAb (CST #3024, 1:1000) detecting pIGF1R (Tyr1135/1136) as well as pINSR (Tyr1150/1151) overnight at 4° C. followed by a 3 hr incubation at room temperature with a mouse mAb detecting Tubulin (loading control) and an additional 1 hr incubation in the dark at room temperature with both Alexa fluor 680 conjugated anti-mouse IgG and IRDye 800CW conjugated anti-rabbit IgG as secondary antibodies. Quantification is performed by densitometry using an Odyssey infrared imager and raw data are processed in an Excel analysis template. The effect of a particular test compound concentration on receptor phosphorylation is expressed relative to the ligand-stimulated control cells (set as 100%), after protein loading normalization assessed by the Tubilin signal. IC50 values are determined using a 4-parametric curve-fitting (XLfit software, v4.3.2; model 205).

In a preferred embodiment, the invention relates to compounds of formula I, which in the above-described “Capture ELISA” assay have an IC₅₀ value of less than 500 nM, most preferably those having an IC₅₀ value of less than 200 nM.

The efficacy of the compounds of the invention (i.e. a compound of formula I as defined herein) as inhibitors of IGF-IR tyrosine kinase activity can be demonstrated as follows: in vivo activity in the nude mouse xenotransplant model.

For in vivo efficacy experiments, cells were resuspended in HBSS were injected subcutaneously (0.05 ml/mouse) into female nude (HsdNpa:athymic/nu) mice 6-8 weeks of age. Treatments were initiated when the mean tumor volumes were approximately 200 mm³. Body weights and tumor volumes were recorded three times a week. Tumor volumes were measured with calipers and determined according to the formula length×diameter²×π/6. In addition to presenting fractional changes of tumor volumes over the course of treatments, antitumor activity is expressed as TIC % (mean change of tumor volume of treated animals/mean change of tumor volume of control animals)×100. Efficacy of candidate IGF-1R inhibitors was determined by initiating oral dosing on day 17-18 post-cell injection following randomization of the mice so that each group has similar mean tumor size. Dosing with an appropriate schedule continued for 7 days based on the general health condition of the animals. All candidate IGF-1R inhibitors were formulated in a suitable vehicle, eg NMP/PEG300 (10:90) and applied daily by gavage. Vehicle consisted of, eg NMP/PEG300 (10:90). All application volumes were 5 ml/kg. After the last measurement a final dose of the compound was given and animals in each treatment group were sacrificed after different time points for terminal PK on blood, liver and other organs, as well as determination of phosphorylated IGF-1R (pIGF-1R) levels in tumor and phosphorylated InsR (pinsR) in liver samples. Plasma insulin levels were assessed using a commercial available ELISA kit (Mercodia). Blood glucose levels were assessed using a glucometer (One Touch Ultra®, LifeScan).

On the basis of these studies, a compound of formula I according to the invention shows therapeutic efficacy especially against proliferative diseases responsive to an inhibition of the IGF-IR tyrosine kinase.

In a further embodiment, the invention relates to a process or a method for the treatment of one of the pathological conditions mentioned hereinabove, especially a disease which responds to an inhibition of the IGF-IR tyrosine kinase or of the IGF-IR-dependent cell proliferation, especially a corresponding neoplastic disease. The compounds of formula I, or a pharmaceutically acceptable salt thereof, can be administered as such or in the form of pharmaceutical compositions, prophylactically or therapeutically, preferably in an amount effective against the said diseases, to a warm-blooded animal, for example a human, requiring such treatment, the compounds especially being used in the form of pharmaceutical compositions. In the case of an individual having a bodyweight of about 70 kg the daily dose administered is from approximately 0.1 g to approximately 5 g, preferably from approximately 0.2 g to approximately 2 g, of a compound of the present invention. In a further embodiment, the invention relates to the use of a compound of formula I, or a pharmaceutically acceptable salt thereof, especially a compound of formula I which is said to be preferred, or a pharmaceutically acceptable salt thereof, as such or in the form of a pharmaceutical composition with at least one pharmaceutically acceptable carrier, for the therapeutic and also prophylactic management of one or more of the diseases mentioned hereinabove, preferably a disease which responds to an inhibition of the IGF-IR tyrosine kinase or of the IGF-IR-dependent cell proliferation, especially a neoplastic disease, in particular if the said disease responds to an inhibition of the IGF-IR tyrosine kinase or of the IGF-IR-dependent cell proliferation.

In a further embodiment, the invention relates to the use of a compound of formula I, or a pharmaceutically acceptable salt thereof, especially a compound of formula I which is said to be preferred, or a pharmaceutically acceptable salt thereof, for the preparation of a pharmaceutical composition for the therapeutic and also prophylactic management of one or more of the diseases mentioned hereinabove, especially a neoplastic disease, in particular if the disease responds to an inhibition of the IGF-IR tyrosine kinase or of the IGF-IR-dependent cell proliferation.

The invention relates in a fourth aspect to pharmaceutical compositions comprising a compound of the present invention. The invention thus provides
a pharmaceutical composition comprising (i.e. containing or consisting of) a compound as defined herein and one or more carriers/excipients;
a pharmaceutical composition comprising a therapeutically effective amount of a compound of formula I as defined herein, and one or more pharmaceutically acceptable carri-ers/excipients.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS

Suitable excipients/carriers may be any solid, liquid, semi-solid or, in the case of an aerosol composition, gaseous excipient that is generally available to one of skill in the art.

Solid pharmaceutical excipients include starch, cellulose, talc, glucose, lactose, sucrose, ge-latin, malt, rice, flour, chalk, silica gel, magnesium stearate, sodium stearate, glycerol monos-tearate, sodium chloride, dried skim milk and the like.

Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of the formula (I) in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, I8th ed., 1990).

The dosage of the active ingredient depends upon the disease to be treated and upon the species, its age, weight, and individual condition, the individual pharmacokinetic data, and the mode of administration. The amount of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. Preferably, the compound is present at a level of about 1-80 wt %. Unit dose forms are, for example, coated and uncoated tablets, ampoules, vials, suppositories or capsules. Examples are capsules containing from about 0.05 g to about 1.0 g of active substance.

Compositions for enteral administration, such as nasal, buccal, rectal or, especially, oral administration, and for parenteral administration, such as intravenous, intramuscular or subcutaneous administration, to warm-blooded animals, especially humans, are especially preferred. The compositions contain the active ingredient alone or, preferably, together with a pharmaceutically acceptable carrier.

Pharmaceutical compositions comprising a compound of formula (I) as defined herein in association with at least one pharmaceutical acceptable carrier (such as excipient a and/or di-luent) may be manufactured in conventional manner, e.g. by means of conventional mixing, granulating, coating, dissolving or lyophilising processes.

In a further embodiment, the invention relates to a pharmaceutical composition for administration to a warm-blooded animal, especially humans or commercially useful mammals suffering from a disease which responds to an inhibition of the IGF-IR tyrosine kinase or of the IGF-IR-dependent cell proliferation, especially a neoplastic disease, comprising an effective quantity of a compound of formula I for the inhibition of the IGF-IR tyrosine kinase or of the IGF-IR-dependent cell proliferation, or a pharmaceutically acceptable salt thereof, together with at least one pharmaceutically acceptable carrier.

In a further embodiment, the invention relates to a pharmaceutical composition for the prophylactic or especially therapeutic management of neoplastic and other proliferative diseases of a warm-blooded animal, especially a human or a commercially useful mammal requiring such treatment, especially suffering from such a disease, comprising as active ingredient in a quantity that is prophylactically or especially therapeutically active against said diseases a new compound of formula I, or a pharmaceutically acceptable salt thereof, is likewise preferred.

The invention relates in a fifth aspect to combinations comprising a compound of formula I and one or more additional active ingredients. The invention thus provides
a combination in particular a pharmaceutical combination, comprising a therapeutically effective amount of a compound of formula I and one or more therapeutically active agents, particularly antiproliferative agents;
a combined pharmaceutical composition, adapted for simultaneous or sequential adminis-tration, comprising a therapeutically effective amount of a compound of formula (I) as defined herein; therapeutically effective amount(s) of one or more combination partners, particularly antiproliferative agents; one or more pharmaceutically acceptable excepients;
a combined pharmaceutical composition as defined herein (i) as pharmaceutical, (ii) for use in the treatment of a IGF-1R mediated disease, (iii) in a method of treatment of a IGF-1R mediated disease.

The term “pharmaceutical combination” or “combined pharmaceutical composition”, as used herein, refers to a product obtained from mixing or combining active ingredients, and includes both fixed and non-fixed combinations of the active ingredients. The term “fixed combination” means that the active ingredients, e.g. a compound of formula (I) and a co-agent, are both administered to a patient simultaneously in the form of a single entity or dosage. The term “non-fixed combination” means that the active ingredients, e.g. a compound of formula (I) and a co-agent, are both administered to a patient as separate entities either simultaneously, concurrently or sequentially with no specific time limits, wherein such administration provides therapeutically effective levels of the active ingredients in the body of the patient. The latter also applies to cocktail therapy, e.g. the administration of three or more active ingredients.

The term “antiproliferative agent” includes, but are not limited to, aromatase inhibitors, antiestrogens, topoisomerase I inhibitors, topoisomerase II inhibitors, microtubule active agents, alkylating agents, histone deacetylase inhibitors, farnesyl transferase inhibitors, COX-2 inhibitors, MMP inhibitors, compounds decreasing the lipid kinase activity, eg PI3 kinase inhibitors, antineoplastic antimetabolites, platin compounds, compounds decreasing the protein kinase activity, eg mTOR inhibitors, Raf inhibitors, MEK inhibitors, and further anti-angiogenic compounds, gonadorelin agonists, anti-androgens, bengamides, bisphosphonates and trastuzumab, radiotherapy.

The term “aromatase inhibitors” as used herein relates to compounds which inhibit the estrogen production, i.e. the conversion of the substrates androstenedione and testosterone to estrone and estradiol, respectively. The term includes, but is not limited to steroids, especially exemestane and formestane and, in particular, non-steroids, especially aminoglutethimide, vorozole, fadrozole, anastrozole and, very especially, letrozole. Exemestane can be administered, e.g., in the form as it is marketed, e.g. under the trademark AROMASIN™. Formestane can be administered, e.g., in the form as it is marketed, e.g. under the trademark LENTARON™. Fadrozole can be administered, e.g., in the form as it is marketed, e.g. under the trademark AFEMA™. Anastrozole can be administered, e.g., in the form as it is marketed, e.g. under the trademark ARIMIDEX™. Letrozole can be administered, e.g., in the form as it is marketed, e.g. under the trademark FEMARA™ or FEMAR™. Aminoglutethimide can be administered, e.g., in the form as it is marketed, e.g. under the trademark ORIMETENT™.

A combination of the invention comprising an antineoplastic agent which is an aromatase inhibitor is particularly useful for the treatment of hormone receptor positive breast tumors.

The term “antiestrogens” as used herein relates to compounds which antagonize the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX™. Raloxifene hydrochloride can be administered, e.g., in the form as it is marketed, e.g. under the trademark EVISTA™. Fulvestrant can be formulated as disclosed in U.S. Pat. No. 4,659,516 or it can be administered, e.g., in the form as it is marketed, e.g. under the trademark FASLODEX™.

The term “topoisomerase I inhibitors” as used herein includes, but is not limited to topotecan, irinotecan, 9-nitrocamptothecin and the macromolecular camptothecin conjugate PNU-166148 (compound A1 in WO99/17804). Irinotecan can be administered, e.g., in the form as it is marketed, e.g. under the trademark CAMPTOSAR™. Topotecan can be administered, e.g., in the form as it is marketed, e.g. under the trademark HYCAMTIN™.

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™. Doxorubicin can be administered, e.g., in the form as it is marketed, e.g. under the trademark ADRIBLASTIN™. Epirubicin can be administered, e.g., in the form as it is mar-keted, e.g. under the trademark FARMORUBICIN™. Idarubicin can be administered, e.g., in the form as it is marketed, e.g. under the trademark ZAVEDOS™. Mitoxantrone can be administered, e.g., in the form as it is marketed, e.g. under the trademark NOVANTRON™.

The term “lipid kinase inhibitors' relates to PI3 kinase inhibitors, PI4 kinase inhibitors, Vps34 inhibitors. Specific examples include: NVP-BEZ235, NVP-BGT226, NVP-BKM120, AS-604850, AS-041164, AS-252424, AS-605240, GDC0941, PI-103, TGX221, YM201636, ZSTK474, examples described in WO 2009/080705 and US 2009/163469.

The term “microtubule active agents” relates to microtubule stabilizing and microtubule destabilizing agents including, but not limited to the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D. Docetaxel can be administered, e.g., in the form as it is marketed, e.g. under the trademark TAXOTERE™. Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTIN™. Discodermolide can be obtained, e.g., as disclosed in U.S. Pat. No. 5,010,099.

The term “alkylating agents” as used herein includes, but is not limited to cyclophosphamide, Ifosfamide and melphalan. Cyclophosphamide can be administered, e.g., in the form as it is marketed, e.g. under the trademark CYCLOSTIN™. Ifosfamide can be administered, e.g., in the form as it is marketed, e.g. under the trademark HOLOXAN™.

The term “histone deacetylase inhibitors” relates to compounds which inhibit the histone deacetylase and which possess antiproliferative activity.

The term “farnesyl transferase inhibitors” relates to compounds which inhibit the farnesyl transferase and which possess antiproliferative activity.

The term “COX-2 inhibitors” relates to compounds which inhibit the cyclooxygenase type 2 enyzme (COX-2) and which possess antiproliferative activity such as celecoxib (Celebrex®) and rofecoxib (Vioxx®).

The term “MMP inhibitors” relates to compounds which inhibit the matrix metalloproteinase (MMP) and which possess antiproliferative activity.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “antineoplastic antimetabolites” includes, but is not limited to 5-fluorouracil, 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY264618 (LOMOTREXOL™) and OGT719.

The term “platin compounds” as used herein includes, but is not limited to carboplatin, cis-platin and oxaliplatin. Carboplatin can be administered, e.g., in the form as it is marketed, e.g. under the trademark CARBOPLAT™. Oxaliplatin can be administered, e.g., in the form as it is marketed, e.g. under the trademark ELOXATIN™.

The term “compounds decreasing the protein kinase activity and further antiangiogenic compounds” as used herein includes, but is not limited to compounds which decrease the activity of e.g. the Vascular Endothelial Growth Factor (VEGF), the Epidermal Growth Factor (EGF), and c-Src and anti-angiogenic compounds having another mechanism of action than decreasing the protein kinase activity.

Compounds which decrease the activity of VEGF are especially compounds which inhibit the VEGF receptor, especially the tyrosine kinase activity of the VEGF receptor, and compounds binding to VEGF, and are in particular those compounds, proteins and monoclonal antibodies generically and specifically disclosed in WO 98/35958 (describing compounds of formula I), WO 00/09495, WO 00/27820, WO 00/59509, WO 98/11223, WO 00/27819, WO 01/55114, WO 01/58899 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, vol. 27, no. 1, pp 14-21, 1999; in WO 00/37502 and WO 94/10202; Angiostatin, described by M. S. O'Reilly et al, Cell 79, 1994, 315-328; and Endostatin, described by M. S. O'Reilly et al, Cell 88, 1997, 277-285; sorefanib (Nexavar), Sutent (sunitinib), BAY 43-9006.

Compounds which decrease the activity of EGF are especially compounds which inhibit the EGF receptors, especially the tyrosine kinase activity of the EGF receptors, and compounds binding to EGF, and are in particular those compounds generically and specifically disclosed in WO 97/02266 (describing compounds of formula IV), EP 0 564 409, WO 99/03854, EP 0520722, EP 0 566 226, EP 0 787 722, EP 0 837 063, WO 98/10767, WO 97/30034, WO 97/49688, WO 97/38983 and, especially, WO 96/33980. Specific EGF receptor inhibitor examples include, but not limited to; Tarceva (erlotinib), Iressa (Gefitinib), Tywerb (lapatanib). Erbitux (cetuximab), Avastin (bevacizumab), Herceptin (trastuzamab), Rituxan (rituximab), Bexxar (tositumomab), panitumumab.

Compounds which decrease the activity of c-Src include, but are not limited to, compounds inhibiting the c-Src protein tyrosine kinase activity as defined below and to SH2 interaction inhibitors such as those disclosed in WO97/07131 and WO97/08193;

compounds inhibiting the c-Src protein tyrosine kinase activity include, but are not limited to, compounds belonging to the structure classes of pyrrolopyrimidines, especially pyrrolo[2,3-d]pyrimidines, purines, pyrazopyrimidines, especially pyrazo[3,4-d]pyrimidines, pyrazopyrimidines, especially pyrazo[3,4-d]pyrimidines and pyridopyrimidines, especially pyrido[2,3-d]pyrimidines. Preferably, the term relates to those compounds disclosed in WO 96/10028, WO 97/28161, WO97/32879 and WO97/49706;

Compounds which decrease the activity of Raf kinases include, but are not limited to: Raf265, sorefanib, BAY 43-9006.

Compounds which inhibit downstream effectors of Raf kinases, such as MEK. Examples of MEK inhibitors include; PD 98059, AZD6244 (ARRY-886), CI-1040, PD 0325901, u0126. Anti-angiogenic compounds having another mechanism of action than decreasing the protein kinase activity include, but are not limited to e.g. thalidomide (THALOMID™), SU5416, and celecoxib (Celebrex™)

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™ Abarelix can be formulated, eg. as disclosed in U.S. Pat. No. 5,843,901.

The term “anti-androgens” as used herein includes, but is not limited to bicalutamide (CASODEX™), which can be formulated, e.g. as disclosed in U.S. Pat. No. 4,636,505.

The term “bengamides” relates to bengamides and derivatives thereof having aniproliferative properties and includes, but is not limited to the compounds generically and specifically disclosed in WO00/29382, preferably, to the compound disclosed in Example 1 of WO00/29382.

The term “bisphosphonates” as used herein includes, but is not limited to etridonic acid, clodronic acid, tiludronic acid, pamidronic acid, alendronic acid, ibandronic acid, risedronic acid and zoledronic acid. “Etridonic acid” can be administered, e.g., in the form as it is marketed, e.g. under the trademark DIDRONEL™. “Clodronic acid” can be administered, e.g., in the form as it is marketed, e.g. under the trademark BONEFOS™. “Tiludronic acid” can be administered, e.g., in the form as it is marketed, e.g. under the trademark SKELID™ “Pamidronic acid” can be administered, e.g., in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANAT™. “Risedronic acid” can be administered, e.g., in the form as it is marketed, e.g. under the trademark ACTONEL™. “Zoledronic acid” can be administered, e.g., in the form as it is marketed, e.g. under the trademark ZOMETA™.

“Trastuzumab” can be administered, e.g., in the form as it is marketed, e.g. under the trademark HERCEPTIN™.

For the treatment of AML, compounds of formula I can be used in combination with standard leukemia therapies, especially in combination with therapies used for the treatment of AML. In particular, compounds of formula I can be administered in combination with e.g. farnesyltransferase inhibitors and/or other drugs used for the treatment of AML, such as Daunorubicin, Adriamycin, Ara-C, VP-16, Teniposide, Mitoxantrone, Idarubicin and Carboplatinum.

The structure of the active agents identified by code nos., generic or trade names may be taken from the actual edition of the standard compendium “The Merck Index” or from databases, e.g. Patents International (e.g. IMS World Publications).

The abovementioned compounds, which can be used in combination with a compound of formula I, can be prepared and administered as described in the art such as in the documents cited above.

In one further embodiment, the additional active ingredient is a hormonal medicine.

### EXAMPLES

The following Examples serve to illustrate the invention without limiting its scope. Abbreviations used are those conventional in the art.

### I Analytical Methods

Temperatures are measured in degrees Celsius. Unless otherwise indicated, the reactions take place at RT. The following HPLC, MS and HPLC/MS methods are used in the preparation of the Intermediates and Examples:

### HPLC Method A

### Column: Speed ROD RP18e, 50×4.6 mm.

Flow rate: 1.3 ml/min
Mobile phase: A) TFA/water (0.11100, v/v), B) TFA/acetonitrile (0.1/100, v/v)
Gradient: linear gradient from 0% B to 100% B in 6 minutes then 2 minutes 100% B

### Detection: UV at 215 nm

### HPLC Method B

Nucleophil Gravity C18 column 70×4 mm, 2 mL/min, 1→20% B in 1 minute, 20→100% B in 2 minutes, A=0.1% TFA in H₂O, B=0.1% TFA in MeCN

### Detection UV

### MS Method L

### Micromass Platform II

### Range DA 200-900, or 100-900

### Cone +30 V and −30 V

Pump Agilent 1100 Quat, 2 minutes, 0.05 ml/min 1:1 methanol: 15% methanol in water, containing 0.2% ammonium hydroxide (25%).

### Injector, CTC PAL

### HPLC/MS Method M

### Agilent G1379A Degasser

### Agilent G1312A Binary Pump

### Agilent G1367A Well Plate Auto Sampler

### Agilent G1316A Column Heater

### Agilent G1315B Diode Array Detector

### Agilent G1496C MSD

### Sedex 75 Evaporative Light Scattering Detector

### Mobile Phase: H2O+0.05% TFA and Acetonitrile+0.035% TFA

Gradient: 1 mL/minute, initial 10% ACN to final 90% ACN in 3 minutes, 100% B for 0.49 mi-nutes, 100% B to initial 10% B in 0.1 minute. The column is re-equilibrated in the ˜45 seconds between injections.
MS Scan: 150 to 1000 amu in 1 second
Diode Array Detector: monitors 220 nm, 254 nm, and 280 nm

### HPLC/MS Method N

### Instrument: ZQ01

### Column: Ascentis Express C-18 2.7 μm, 2.1×30 mm

### Column-Temp: 50° C.

Eluent A: Water+0.05% formic acid+0.05% ammonium acetate
Eluent B: acetonitrile+0.04% formic Acid
Flow: 0.9 ml/min

### Gradient: Time % B

0.00 2
1.70 98
2.15 98
2.19 2

### UV: 210-350 nm

### MS: 100-1200 m/z (Cone 17 V/Cap. pos. 3.25 kV neg. 3.5 kV)

### Preparative HPLC

### Method R

Gilson preparative HPLC system, with UV-triggered collection system
Column, Sunfire Prep C18 OBD 5 microm 30×100 mm, temperature 25° C.
Eluent, gradient from 5-100% acetonitrile in 0.05% aqueous trifluoroacetic acid over 20 minutes, flow rate 30 ml/min.

Detection UV 254 nm

### Method S

### Instrument: Waters 2525 Binary Pump, Waters 515 Make Up Pump, Waters 2757 Auto Sampler/Fraction Collector, Waters 2487 Dual Wavelength UV Detector, Waters ZQ Mass Spectrometer

Mass triggered collection system.

### Mobile Phase: H2O+0.05% TFA (A), Acetonitrile+0.035% TFA (B)

### UV Detector: 220 nm and 254 nm

MS Scan: 180 to 800 amu in 0.5 seconds

### Gradient:

| Time (min) | Flow Rate (mL/min) | % B |
|---|---|---|
| 0 | 20 | 10 |
| 1.4 | 20 | 10 |
| 1.45 | 100 | 10 |
| 3.99 | 100 | 40 |
| 4 | 100 | 100 |
| 4.15 | 100 | 100 |
| 4.16 | 100 | 10 |
| 4.2 | 10 | 10 |
| 4.25 | 10 | 10 |

### HPLC/MS Method X

### Instrument: ZQ 2000

Range Da 100-900 (positive) and 120-900 (negative)

### Cone +17 V and −17 V

Pump Agilent 1100 Bin, 3.5 minute run time, channel A water with 5% acetonitrile, channel B acetonitrile, containing 0.5-1.0% formic acid

| Time (mins) | Flow Rate (mL/min) | % B |
|---|---|---|
| 0 | 1.2 | 10 |
| 1.4 | 1.4 | 95 |
| 1.45 | 2.4 | 95 |
| 3.99 | 2.4 | 10 |

Injector, CTC PAL, 5 microl

### Oven Agilent 1100, 50° C.

Column, Waters XBridge, 3×30 mm, 2.5 microm, C18

### Detector, Agilent 1100 DAD, 210-350 nm

### HPLC/MS Method Y

Instrument: Agilent G1379A Degasser, Agilent G1312A Binary Pump, Agilent G1367A Well Plate Auto Sampler, Agilent G1316A Column Heater, Agilent G1315B Diode Array Detector, Agilent G1496C MSD, Sedex 75 Evaporative Light Scattering Detector

### Eluent:

A: Water+0.05% Formic Acid+0.05% Ammonium acetate (7.5 M solution)

### B: Acetonitrile+0.04% Formic Acid

### Column

### Ascentis Express RP-Amide 2.7 um 2.1×30 mm @ 50° C.

### Gradient

Flow: 1.2 ml/min

| Time | % B |
|---|---|
| 0 | 2 |
| 1.7 | 98 |
| 2.15 | 98 |
| 2.19 | 2 |

UV detection, DAD 210-350 nm
MS detection, 100-900 m/z

### HPLC/MS Method Z

### Instrument: ZQ 2000

Range Da 100-900 (positive) and 120-900 (negative)

### Cone +17 V and −17 V

Pump Agilent 1100 Bin, 3.5 minute run time, channel A water with 5% acetonitrile, channel B acetonitrile, containing 0.5-1.0% formic acid

| Time (mins) | Flow Rate (mL/min) | % B |
|---|---|---|
| 0 | 1.2 | 10 |
| 1.70 | 1.4 | 95 |
| 2.40 | 2.4 | 95 |
| 2.45 | 2.4 | 10 |

Injector, CTC PAL, 5 microl

### Oven Agilent 1100, 50° C.

Column, Waters XBridge, 3×30 mm, 2.5 microm, C18

### Detector, Agilent 1100 DAD, 210-350 nm

II Chemical Synthesis—Intermediates

### Intermediates

### Intermediate A: (R,S)-2-Phenyl-7-(4,4,5,5-tetramethyl-[1,3,2]dioxaborolan-2-yl)chroman

A mixture of (R,S)-trifluoromethanesulfonic acid 2-phenyl-chroman-7-yl ester (Intermediate B, 3.6 g, 9.9 mmol), bis(pinacolato)diboron (2.8 g, 10.8 mmol), 1,1-bis(diphenylphosphino)ferrocene (0.17 g, 0.30 mmol), 1,1-bis(diphenylphosphino)-ferrocenedichloropalladium(II) dichloromethane complex (0.22 g, 0.30 mmol) and potassium acetate (2.4 g, 24.6 mmol) in 1,4-dioxan (13 ml) was sealed under an argon atmosphere and heated with stirring at 100° C. for 5 hours in a Biotage microwave apparatus. The cooled reaction mixture was partitioned between water and ethyl acetate, the organic layers washed with brine, dried over sodium sulphate and evaporated to give the crude product which was suspended in methanol and filtered to give the title compound as a pink solid. HPLC/MS (Method Z) t_{R} 1.77 minute, M+H 337.1 (20%).

### Intermediate B: (R,S)-Trifluoromethanesulfonic acid 2-phenyl-chroman-7-yl ester

Triflic anhydride (2.0 ml, 12.1 mmol) was added to a stirred mixture of (R,S)-2-phenyl-chroman-7-ol (Intermediate C, 2.4 g, 10.1 mmol), 2,6-lutidine (2.3 ml, 202.2 mmol) and DMAP (0.3 g, 2.5 mmol) in dichloromethane (70 ml) at 0° C. After stirring for 1 hour 1M aqueous HCl was added and extracted twice with dichloromethane. The combined organic layers were washed with 1M aqueous HCl, then aqueous NaHCO₃ and dried over sodium sulphate. Evaporation gave the title compound as a brown solid. ¹H NMR (600 MHz, CDCl₃) δ ppm 7.44-7.41 (m, 4H), 7.39-7.34 (m, 1H), 7.14 (d, 1H), 6.86 (d, 1H), 6.81 (dd, 1H), 5.08-5.05 (m, 1H), 3.03-2.94 (m, 1H), 2.85-2.76 (m, 1H), 2.32-2.28 (m, 1H), 2.19-2.08 (m, 1H).

### Intermediate C: (R,S)-2-Phenyl-chroman-7-ol

10% Palladium on carbon (3.57 g) was added to a mixture of 7-hydroxyflavone (8 g, 33.6 mmol) and ethyl acetate (300 ml) at room temperature under a nitrogen atmosphere. An atmosphere of hydrogen was introduced and the mixture stirred under 1 atmosphere of hydrogen for 45 hours. The reaction mixture was filtered through a 0.2 μM Fluoropore Membrane Filter and the solvent removed in vacuo. Purification of the residue by silica gel flash column chromatography, eluting with a gradient of ethyl acetate in heptane, gave the title compound as a colourless oil. MS (Method L) M+H 227.1 (100%) and M−H 225.2 (100%).

### Intermediate D: (R,S)-2-Phenyl-7-(4,4,5,5-tetramethyl-[1,3,2]dioxaborolan-2-yl)-2H-chromene

A mixture of (R,S)-7-bromo-2-phenyl-2H-chromene (Intermediate E, 2.5 g, 8.5 mmol), bis(pinacolato)diboron (2.8 g, 10.8 mmol), 1,1-bis(diphenylphosphino)-ferrocenedichloropalladium(II) dichloromethane complex (0.62 g, 0.85 mmol) and potassium acetate (2.1 g, 21.1 mmol) in DMF (9 ml) was sealed under an argon atmosphere and heated with stirring at 80° C. for 15 hours. The cooled reaction mixture was partitioned between water and dichloromethane, the organic layers were dried over sodium sulphate and evaporated to give the crude product which was purified by silica gel flash column chromatography, eluting with a gradient of methanol in dichloromethane, to give the title compound as a clear yellow oil. HPLC/MS (Method Z) t_{R} 1.75 minute, M+H 334.8 (100%).

### Intermediate E: (R,S)-7-Bromo-2-phenyl-2H-chromene

Following the procedure of Wang and Finn Org. Lett. 2000, 2, 4063-4065. A stirred mixture of 4-bromo-2-hydroxybenzaldehyde (8.4 g, 41.8 mmol), trans-2-phenylvinylboronic acid (8.8 g, 59.3 mmol) and dibenzylamine (6.7 ml, 41.8 mmol) in 1,4-dioxane (300 ml) was heated at 95° C. for 15 hours. The cooled reaction mixture was then partitioned between water and dichloromethane, the organic layers washed with 1M aqueous HCl, then aqueous sodium bicarbonate solution and dried over sodium sulphate. Purification of the residue by silica gel flash column chromatography, eluting with a gradient of ethyl acetate in heptane, gave the title compound as a yellow/white crystalline solid. HPLC/MS (Method Z) t_{R} 1.67 minute, M+H 286.7 and 288.9 (100%).

### Intermediate F: 2-Phenyl-7-(4,4,5,5-tetramethyl-[1,3,2]dioxaborolan-2-yl)-4H-chromene

A mixture of 7-bromo-2-phenyl-4H-chromene (Intermediate G, 0.3 g, 1.0 mmol), bis(pinacolato)diboron (0.35 g, 1.4 mmol), 1,1-bis(diphenylphosphino)-ferrocenedichloropalladium(II) dichloromethane complex (0.1 g, 0.1 mmol) and potassium acetate (0.1 g, 1.0 mmol) in DMF (2 ml) was sealed under an argon atmosphere and heated with stirring at 80° C. for 15 hours. The cooled reaction mixture was partitioned between water and ethyl acetate, the organic layers were washed with brine, dried over magnesium sulphate and evaporated to give the crude product which was purified by silica gel flash column chromatography, eluting with a gradient of ethyl acetate in hexane, to give the title compound as a brown glass. HPLC/MS (Method Z) t_{R} 1.62 minute, M+H 335.0 (100%).

### Intermediate G: 7-Bromo-2-phenyl-4H-chromene

Aluminium chloride (55.7 g, 418 mmol) was added portion-wise to a suspension of lithium aluminium hydride (4.3 g, 114 mmol) in dry THF (1 l) cooled with an ice bath, (caution exo-thermic). A solution of 7-bromo-2-phenyl-chromen-4-one (Intermediate H, 11.4 g, 38.0 mmol) in THF (150 ml) was added dropwise at 0° C. and 10 minutes after the end of the addition ethyl acetate was added, initially with caution until all the organometalic intermediates were quenched. Partitioning between ethyl acetate and water was followed by washing of the combined organic layers with brine and drying over sodium sulphate. Removal of the solvent in vacuo gave the crude product which was crystallized from methanol to give the title compound as a brown solid. HPLC/MS (Method Z) t_{R} 1.64 minute, M+H 287.0 and 289.1 (100%).

### Intermediate H: 7-Bromo-2-phenyl-chromen-4-one

A mixture of (E)-1-(4-bromo-2-hydroxy-phenyl)-3-phenyl-propenone (Intermediate I, 14.0 g, 46.2 mmol) and iodine (12.9 g, 50.9 mmol) in triethyleneglycol (67 ml) was stirred at 150° C. for 5 hours. The cooled reaction mixture was then partitioned between water and ethyl ace-tate, the organic layers washed with aqueous sodium thiosulphate, then brine and dried over magnesium sulphate. Purification of the residue by trituration with methanol gave the title compound as a brown solid. ¹H NMR (400 MHz, CDCl₃) δ ppm 8.10 (d, 1H), 7.91 (dd, 2H), 7.79 (d, 1H), 7.49-7.63 (m, 4H), 6.82 (s, 1H).

### Intermediate I: (E)-1-(4-Bromo-2-hydroxy-phenyl)-3-phenyl-propenone

Following the procedure of Nam et al Eur. J. Med. Chem. 2003, 38, 179-187. A stirred mixture of 4-bromo-2-hydroxyacetophenone (10 g, 46.5 mmol), benzaldehyde (5.0 ml, 48.8 mmol) and barium hydroxide octahydrate (29.3 g, 93 mmol) in methanol (300 ml) was heated at 40° C. for 16 hours. The volume of the cooled reaction mixture was then reduced in vacuo and 6N aqueous HCl added. The resulting mixture was then extracted with ethyl acetate, the organic layers washed with water and dried over sodium sulphate. Removal of the solvent in vacuo gave the title compound as a yellow oil. ¹H NMR (400 MHz, CDCl₃) δ ppm 12.94 (s, 1 H), 7.95 (d, 1H), 7.77 (d, 1H), 7.77 (d, 1H), 7.67 (dd, 2H), 7.58 (d, 1H), 7.42-7.49 (m, 2 H), 7.24 (d, 1H), 7.09 (dd, 1H).

### Intermediate J: [cis-3-(4-Amino-5-iodo-pyrrolo[2,3-d]pyrimidin-7-yl)-cyclobutyl]-methanol

The title compound is prepared as described in WO 2005/097800. Or alternatively as described below: A mixture of [3-(4-chloro-5-iodo-pyrrolo[2,3-d]pyrimidin-7-yl)-cyclobutyl]-methanol (Step J.1, 2.0 g, 5.50 mmol), 25% aqueous ammonia solution (10.4 ml) and 1,4-dioxane (5 ml) were heated in sealed tube at 80° C. for 15.5 hours. After cooling the reaction mixture was evaporated and purified by flash column chromatography, eluting with a gradient of DCM/methanol, to give the title compound as an off-white solid. ¹H-NMR (d₆-DMSO, 400 MHz): δ ppm 8.06 (s, 1H), 7.68 (s, 1H), 6.57 (broad s, 2H), 5.06-4.87 (m, 1H), 4.57 (t, 1H), 3.49-3.40 (m, 2H), 2.45-2.35 (m, 2H), 2.28-2.13 (m, 2H).

### Step J.1: [cis-3-(4-Chloro-5-iodo-pyrrolo[2,3-d]pyrimidin-7-yl)-cyclobutyl]-methanol

A solution of DIBAL-H in toluene (0.73 ml, 0.73 mmol) was added dropwise to a stirred suspension of benzoic acid 3-(4-chloro-5-iodo-pyrrolo[2,3-d]pyrimidin-7-yl)-cyclobutylmethyl ester (Intermediate K, 170 mg, 0.36 mmol) in DCM (3 ml) cooled with a dry-ice/acetone bath. After 30 minutes the reaction mixture was warmed over 1 hour to 0° C., stirred 1 hour at 0° C., and silica gel (2 g) was added. The reaction mixture was evaporated and the residue purified by flash chromatography, to give the title compound. HPLC/MS t_{R} 1.09 min, M+H 365.8 (Method X).

### Intermediate K: Benzoic acid cis-3-(4-chloro-5-iodo-pyrrolo[2,3-d]pyrimidin-7-yl)-cyclobutylmethyl ester

A mixture of benzoic acid 3-(4-chloro-pyrrolo[2,3-d]pyrimidin-7-yl)-cyclobutylmethyl ester (Step K.1, 1.7 g, 4.97 mmol), N-iodosuccinimide (NIS) (1.23 g, 5.47 mmol) and DMF (9 ml) was stirred at room temperature for 48 hours. Ethyl acetate and water were added and the title compound collected by filtration. HPLC/MS t_{R} 3.46 min, M+H 468.2 and M−H 467.0 (Method Y).

### Step K.1: Benzoic acid cis-3-(4-chloro-pyrrolo[2,3-d]pyrimidin-7-yl)-cyclobutylmethyl ester

A mixture of (4,6-dichloro-pyrimidin-5-yl)-acetaldehyde (Astatech, 1.40 g, 7.31 mmol), benzoic acid 3-amino-cyclobutylmethyl ester (prepared as described in Org. Process Res. Dev. 2007, 11, 825-835., 1.5 g, 7.31 mmol), diisopropylethylamine (0.95 g, 7.31 mmol) and ethanol (15 ml) were heated at reflux for 5.5 hours under an argon atmosphere. The reaction mixture was evaporated, taken up in THF (10 ml), aqueous HCl (4 ml, 4M) added and stood at room temperature for 1 hour. The volume of the mixture was then reduced under vacuum, made neutral with aqueous sodium bicarbonate solution, extracted 3× with DCM, the organic layers dried over sodium sulphate and evaporated. Purification by flash column chromatography, eluting with a DCM/EtOAc gradient gave the title compound. HPLC/MS t_{R} 1.52 min, M+H 342.1 (Method X).

### Intermediate L: [Trans-3-(4-amino-5-iodo-pyrrolo[2,3-d]]pyrimidin-7-yl)-cyclobutyl]-methanol

The title compound was prepared in a similar manner to Intermediate J. MS m/k 345 (M+H⁺) (Method M).

### Intermediate M: (R,S)-cis-3-[8-Amino-1-(2-phenyl-chroman-7-yl)-imidazo[1,5-a]pyrazin-3-yl]-cyclobutanecarbaldehyde

(R,S)-{cis-3-[8-Amino-1-(2-phenyl-chroman-7-yl)-imidazo[1,5-a]pyrazin-3-yl]-cyclobutyl}-methanol (Example 3) (40 mg, 0.09 mmol) was dissolved in acetonitrile (5 mL) at room temperature. 2-Iodoxybenzoic acid (IBX) (70 mg, 0.11 mmol) was added, the flask purged with argon, sealed and the reaction allowed to stir at 70° C. for 40 min. After cooling to room tem-perature, volatiles were removed under reduced pressure. The isolated product was used in the following reactions without further purification. HPLC/MS (Method Z) t_{R} 1.23 minute, M+H 426.3 (80%).

### Intermediate N: (Z)-3-(8-Amino-1-iodo-imidazo[1,5-a]pyrazin-3-yl)-1-hydroxymethyl-cyclobutanol and (E)-3-(8-amino-1-iodo-imidazo[1,5-a]pyrazin-3-yl)-1-hydroxymethyl-cyclobutanol

The title compound is obtained by analogy to the procedure described for Intermediate J from a mixture of (Z)-3-(8-chloro-1-iodo-imidazo[1,5-a]pyrazin-3-yl)-1-hydroxymethyl-cyclobutanol and (E)-3-(8-chloro-1-iodo-imidazo[1,5-a]pyrazin-3-yl)-1-hydroxymethyl-cyclobutanol (WO 2005/097800). M+H 362.2 (Method X). NMR (400 MHz, DMSO-d6) δ ppm 7.43 (d, 1H), 6.94 (d, 1H), 6.57 (bs, 2H), 3.85-3.76 (m, 1H), 3.15 (s, 2H), 2.49-2.42 (m, 2H), 2.25-2.21 (m, 2H).

### Intermediate O: 5-Bromo-7-[cis-3-(1,1-dioxo-thiomorpholin-4-ylmethyl)-cyclobutyl]-7H-pyrrolo[2,3-d]pyrimidin-4-ylamine

A mixture of 5-bromo-4-chloro-7-[cis-3-(1,1-dioxo-thiomorpholin-4-ylmethyl)-cyclobutyl]-7H-pyrrolo[2,3-d]pyrimidine (Step O.1, 5.2 g, 11.99 mmol) and ammonium hydroxide (52.1 g, 372 mmol) was dissolved in dioxane (58 ml) and stirred for 13 hours at 100° C. under pres-sure. After cooling and evaporation of the solvent the solid residue was washed with methanol (50 ml), filtered and dried to give the title compound as an off-white solid. HPLC/MS (Method N) t_{R} 0.65 minute, M+H 414.3/416.3 (100%).

### Step O.1: 5-Bromo-4-chloro-7-[cis-3-(1,1-dioxo-thiomorpholin-4-ylmethyl)-cyclobutyl]-7H-pyrrolo[2,3-d]pyrimidine

A solution of 4-chloro-7-[cis-3-(1,1-dioxo-thiomorpholin-4-ylmethyl)-cyclobutyl]-7H-(Step O.2, 4.65 g, 13.1 mmol) and N-Bromosuccinimide (3.06 g, 17.04 mmol) were dissolved in DMF (47 ml) and stirred 3 hours at room temperature. The volume of the reaction mixture was then reduced in vacuo and after addition of water and extraction of the product with DCM pure title compound was obtained from crystallization in DCM. HPLC/MS (Method N) t_{R} 1.08 minute, M+H 433.3/435.3 (100%).

### Step O.2: 4-Chloro-7-[cis-3-(1,1-dioxo-thiomorpholin-4-ylmethyl)-cyclobutyl]-7H-pyrrolo[2,3-d]pyrimidine

A solution of cis-3-(1,1-dioxo-thiomorpholin-4-ylmethyl)-cyclobutylamine (Step O.3, 7.75 g, 35.5 mmol), (4,6-dichloro-pyrimidin-5-yl)-acetaldehyde (8.04 g, 40.8 mmol) and Huenig's Base (9.18 g, 71 mmol) were dissolved in ethanol (180 ml) and stirred during 5 hours at 90° C. After cooling to room temperature TFA (40.5 g, 355 mmol) was added and the reaction mixture stirred for another hour at room temperature. After evaporation and extraction with DCM the product was purified by flash chromatography, eluting with a gradient of DCM/methanol, to give the title compound. HPLC/MS (Method N) t_{R} 0.88 minutes, M+H 355.4 (100%).

### Step O.3: cis-3-(1,1-Dioxo-thiomorpholin-4-ylmethyl)-cyclobutylamine

[cis-3-(1,1-Dioxo-thiomorpholin-4-ylmethyl)-cyclobutyl]-carbamic acid benzyl ester (Step O.4, 13.0 g, 36.9 mmol) was dissolved in Ethanol (350 ml) in an appropriate hydrogenation flask, Pd/C (1.295 g, 10%) was added and then hydrogenated at room temperature for 96 hours. Filtration and evaporation gave the title compound as a clear oil. HPLC/MS (Method N) t_{R} 0.11 minute, M+H 219.1 (100%).

### Step O.4: [cis-3-(1,1-Dioxo-thiomorpholin-4-ylmethyl)-cyclobutyl]-carbamic acid benzyl ester

(cis-3-Formyl-cyclobutyl)-carbamic acid benzyl ester (Step O.5, 12.915 g, 53.2 mmol) was dissolved in THF (150 ml), thiomorpholine-1,1-dioxide (14.66 g, 106 mmol) and sodium triacetoxy borohydride (37.6 g, 159 mmol) were added and the mixture was stirred at room temperature for 2 hours. After quenching with sodium bicarbonate solution, extraction with DCM, evaporation, and flash chromatography purification with DCM/methanol gradient, the title compound was obtained as colorless powder. HPLC/MS (Method N) t_{R} 0.87 minute, M+H 353.4 (100%).

### Step O.5: (cis-3-Formyl-cyclobutyl)-carbamic acid benzyl ester

(cis-3-Hydroxymethyl-cyclobutyl)-carbamic acid benzyl ester (Step O.6, 12.564 g, 53.4 mmol) was dissolved in DCM (130 ml) and cooled to −78° C. While cooling was maintained, DMSO (12.52 g, 160 mmol), triethylamine (18.91 g, 187 mmol) and oxalyl chloride (8.47 g, 64.1 mmol) were added. After stirring 1 hour at −78° C. and another hour at room temperature, the reaction mixture was quenched with sodium bicarbonate solution, the product extracted with DCM, evaporated and the crude title compound was used in the next step without further purification. HPLC/MS (Method N) t_{R} 0.92 minute, M+H 234.1 (100%).

### Step O.6: (cis-3-Hydroxymethyl-cyclobutyl)-carbamic acid benzyl ester

Benzoic acid 3-benzyloxycarbonylamino-cyclobutylmethyl ester (Step O.7, 20.2 g, 59.5. mmol) was dissolved in THF (500 ml) and lithium hydroxide (179 ml, 1 molar solution in water) was added. After stirring 16 hours at 50° C., the mixture was extracted with ethyl acetate. Crystallization from DCM/Heptane yielded pure title compound. HPLC/MS (Method N) t_{R} 0.65 minute, M+H 236.1 (100%).

### Step O.7: cis-Benzoic acid 3-benzyloxycarbonylamino-cyclobutylmethyl ester

cis-Benzoic acid 3-amino-cyclobutylmethyl ester (prepared as described by Slade et al Org. Proc. Res. Dev. 2007, 11, 825-835., 15 g, 73.1 mmol) was dissolved in DCM (225 ml). Chlorocarbonic acid monobenzyl ester (18.7 g, 110 mmol) and Huenig's Base (18.89 g, 146 mmol) were added at 0° C. and then stirred for 20 hours at room temperature. After evaporation and flash chromatography with a gradient of DCM/methanol the title compound was obtained. HPLC/MS (Method N) t_{R} 1.17 minute, M+H 340.1 (100%).

### Intermediate P: (R,S)-Toluene-4-sulphonic acid cis-3-[8-amino-1-(2-phenyl-chroman-7-yl)-imidazo[1,5-a]pyrazin-3-yl]-cyclobutylmethyl ester

(R,S)-{cis-3-[8-Amino-1-(2-phenyl-chroman-7-yl)-imidazo[1,5-a]pyrazin-3-yl]-cyclobutyl}-methanol (Example 3) (150 mg, 0.35 mmol) was dissolved in pyridine (1 mL) and cooled to −25° C. At this temperature p-toluenesulfonyl chloride (1.6 g, 1.4 mmol) was added in small portions over 30 min. Stirring at −25° C. was continued for a further 12 hours and the cold reaction mixture then quenched by addition of cold water and DCM. The organic layer was separated and repeatedly washed with ice-cold aqueous 1N sulfuric acid solution. The organic layer was then dried and concentrated under reduced pressure to give the title compound as a yellow foam. HPLC/MS (Method Z) t_{R} 1.21 minutes, M+H 581.2 (80%).

### Intermediates Q and R: (E)-3-(4-Amino-5-iodo-pyrrolo[2,3-d]pyrimidin-7-yl)-1-hydroxymethyl-cyclobutanol and (Z)-3-(4-amino-5-iodo-pyrrolo[2,3-d]pyrimidin-7-yl)-1-hydroxymethyl-cyclobutanol

A mixture (2:1) of E- and Z-3-(4-chloro-5-iodo-pyrrolo[2,3-d]pyrimidin-7-yl)-1-hydroxymethyl-cyclobutanol (Step Q and R.1, 6 g, 15.8 mmol) was suspended in a mixture of dioxane (30 mL) and aqueous NH₃ (25%, 60 mL) and transferred into three glass autoclaves vessels (50 mL) (Büchi, Flawil) and stirred at 100° C. for 19 hours. The combined reaction mixtures were concentrated under reduced pressure to give the crude reaction mixture of intermediates Q and R. This isolated mixture of both isomers was used for the next reaction step without further purification. MS (Method L) M+H=361 (100%). HPLC (Method B): t_{R} 1.83 minutes. TLC (NH₃/MeOH/CH₂Cl₂=1:10:89): R^{E}=0.33 and 0.31, ¹H-NMR (600 MHz, DMSO-d₆): δ ppm (peak intensities Z/E=1:2) 8.08 (E)/8.07 (Z) (s/s, 1H), 7.74 (Z)/7.58 (E) (s/s, 1H), 6.65 (s/broad, 2H), 5.30/4.80 (t/t, 1H), 5.26/5.13 (s/s, 1H), 4.84 (m, 1H), 3.30 (m, 2H), 2.70/2.30 (t/t, 2H, Z-isomer), 2.60/2.25 (t/t, 2H, E-isomer).

### Step Q and R.1: E- and Z-3-(4-Chloro-5-iodo-pyrrolo[2,3-d]pyrimidin-7-yl)-1-hydroxymethyl-cyclobutanol

3-(4-Chloro-pyrrolo[2,3-d]pyrimidin-7-yl)-1-hydroxymethyl-cyclobutanol (Step Q and R.2, 6.94 g, 27.4 mmol) and NIS (7.39 g, 32.8 mmol) was dissolved in DMF (110 mL) and the mixture stirred at 60° C. under argon. After 2.5 hours, NIS (0.25 g, 1.1 mmol) was added and the reaction mixture stirred for a further 1 hour at 60° C. After concentration of the reaction mixture under reduced pressure, sodium bicarbonate solution (15 mL) was added and the resulting suspension was extracted with AcOEt (30 mL, 8×). The combined organic phases were washed with Na₂SO₃ solution (10 mL, 2×) and brine (5 mL, 2×), dried (MgSO₄), and concentrated under reduced pressure to give a beige solid, which was further suspended in hexane and washed, and then dried under vacuum to give the title compound as a beige solid. NS (Method L) M+H=380/382. HPLC (Method B): t_{R} 2.53 min. ¹H-NMR (600 MHz, DMSO-d₆): δ ppm (peak intensities Z/E=1:2) 8.61 (E)/8.59 (Z) (s/s, 1H), 8.25 (Z)/8.12 (E) (s/s, 1H), 5.40/4.86 (quint/quint, 1H), 5.29/5.16 (s/s, 1H), 4.80 (m, 1H), 3.39/3.30 (d/d, 2H), 2.70/2.30 (t/t, 2H/Z-isomer), 2.60/2.25 (t/t, 2H/E-isomer).

### Step Q and R.2: 3-(4-Chloro-pyrrolo[2,3-d]pyrimidin-7-yl)-1-hydroxymethyl-cyclobutanol (2,4-Dichloro-pyrimidin-5-yl)-acetaldehyde (7.21 g, 37.7 mmol), 3-amino-1-hydroxymethyl-cyclobutanol

(Step Q and R.3, 4.42 g, 37.7 mmol), and DIPEA (13.18 mL, 75 mmol) were dissolved in EtOH (190 mL) and stirred under reflux (oil bath at 90° C.) for 4.5 hours. After cooling to room temperature, TFA (260 mmol, 20 mL) was added and the reaction mixture stirred under reflux for a further 1 hour. After cooling to room temperature, conc. NaHCO₃ solution (0.5 L) was added, the alcohol evaporated under reduced pressure, and the reaction mixture was then extracted with AcOEt (4×, 100 mL). The combined organic phases were washed with conc. NaHCO₃ solution (50 mL) and brine (40 mL), dried (MgSO₄), concentrated under reduced pressure, purified by normal phase chromatography on a 120 g RediSept silica gel column, and fractioned by means of a Sepacore Control chromatography system (Büchi, Flawil, Switzerland) (eluent: 1-10% MeOH (10% NH₃) in CH₂Cl₂) to give the title compound as a beige solid. MS (Method L) M+H=254/256 (100%). HPLC (Method B): t_{R} 2.24 min. ¹H-NMR (600 MHz, DMSO-d₆): 6 ppm (peak intensities Z/E=1:2) 8.63 (E)/8.60 (Z) (s/s, 1H), 8.02 (4/7.89 (E) (did, 1H), 6.72 (Z)/6.68 (E) (did, 1H), 3.41/2.78 (quint/quint, 1H), 3.30 (S/broad, 4H), 3.21/3.14 (d/d, 2H), 2.29/1.50 (m/m, 2H/Z-isomer), 1.95/1.70 (t/t, 2H/E-isomer).

### Step Q and R.3: 3-Amino-1-hydroxymethyl-cyclobutanol

(3-Hydroxy-3-hydroxymethyl-cyclobutyl)-carbamic acid benzyl ester (Step Q and R.4, 9.49 g, 37.8 mmol) was dissolved in THF/MeOH (1:1, 150 mL) and hydrogenated under 1 atmosphere of hydrogen for 1 hour in the presence of Pd/C Engelhard 4505 (1.5 g). The reaction mixture was then filtered and the solvent evaporated under reduced pressure to give the title compound as a brown oil. ¹H-NMR (400 MHz, DMSO-d₆): δ ppm (peak intensities Z/E=1:2) 5.40/4.86 (quint/quint, 1H), 5.29/5.16 (s/s, 1H), 4.85 (m, 1H), 3.41/3.31 (d/d, 2H), 2.75/2.40 (t/t, 2H/Z-isomer), 2.68/2.30 (t/t, 2H/E-isomer).

### Step Q and R.4: (3-Hydroxy-3-hydroxymethyl-cyclobutyl)-carbamic acid benzyl ester

(3-Methylene-cyclobutyl)-carbamic acid benzyl ester (Step Q and R.5, 9.915 g, 45.6 mmol) dissolved in tert.-butanol/H₂O (40 mL, 1:1) was added to a solution of AD-Nix alpha (70 g, 50.2 mmol) in tert.-butanol/H₂O (360 mL, 1:1) at 0° C. After stirring at room temperature for 16 hours, the reaction mixture was cooled to 0° C. and Na₂SO₃ (40 g) was added and the reaction mixture was stirred for a further 1 hour at room temperature. After adding H₂O (150 mL), the reaction mixture was extracted with AcOEt (150 mL, 3×). The combined organic phases were washed with brine, dried (MgSO₄), and concentrated under reduced pressure to give the title compound as white solid. MS (Method L): M+H=252. HPLC (Method B): t_{R} 2.32 minutes. TLC (NH₃/MeOH/CH₂Cl₂=1:10:89): R^{F}=0.25. ¹H-NMR (600 MHz, DMSO-d₆): δ ppm (peak intensities Z/E=1:2) 7.51 (Z)/7.44 (E) (s/s, 1H), 7.35 (m, 5H), 4.95 (s, 2H), 4.80 (Z)/4.70 (E) (s/s, 1H), 4.65/4.62 (t/t, 1H), 4.12 (E)/3.52 (Z) (sextet/sextet, 1H), 3.25 (Z)/3.20 (E) (did, 2H), 2.30/1.80 (t/t, 2H/Z-isomer), 1.96 (t, 2H/E-isomer).

### Step Q and R.5: (3-Methylene-cyclobutyl)-carbamic acid benzyl ester

Diphenylphosphoryl azide (25.3 g, 89 mmol) was added to 3-methylene cyclobutyl carboxylic acid (10 g, 89 mmol) and NEt₃ (15 mL, 105 mmol) dissolved in dioxane/MeCN (15 mL/35 mL) over 15 minutes. The temperature of the reaction mixture then increased to 75° C. with the evolution of gas. After heating the reaction mixture for a further 1 hour at 100° C., benzyl alcohol (20 mL) was added and the reaction mixture was then stirred for 19 hours at 100° C. After cooling and evaporation of the solvent, the residue was taken up in AcOEt (250 mL) and extracted with half conc. NH₄Cl solution (80 mL), half con. NaHCO₃ solution (80 mL), and brine (40 mL), dried (MgSO₄), and concentrated under vacuum. The residue was purified by means of a 120 g RediSept silica gel column using a Sepacore Control chromatographic separator (Büchi) (eluent: hexane/AcOEt=1:9→4:6) to give the title compound as a white solid. MS (Method L) M+H=218. HPLC (Method B): t_{R} 3.12 minutes. TLC (AcOEt/hexane=1:4): R^{F}=0.30. ¹H-NMR (400 MHz, DMSO-d₆): δ ppm 7.64 (d, 1H), 7.32 (m, 5H), 4.99 (s, 2H), 4.76 (s, 2H), 3.95 (sextet, 1H), 2.85 (m, 2H), 2.62 (m, 2H).

Alternatively, to obtain the individual geometric isomers (Intermediates Q and R) a separation can be made in the following manner.

The mixture (2:1) of the geometric isomeres E- and Z-3-(4-amino-5-iodo-pyrrolo[2,3-d]pyrimidin-7-yl)-1-hydroxymethyl-cyclobutanol (Intermediates Q and R: 34.5 g, 82.5 mmol) was recrystallized several times from methanol to afford Intermediate Q (E-isomer) as yellow crystals. The mother liquids were combined, concentrated and dried in vacuo to afford the enriched Intermediate R (Z-isomer) as beige crystals.

Alternatively, recrystallisation of the isolated E:Z mixture of Intermediates Q and R from acetic acid gives (E)-3-(4-amino-5-iodo-pyrrolo[2,3-d]pyrimidin-7-yl)-1-hydroxymethyl-cyclobutanol (Intermediate Q) as a white solid. ¹H NMR (400 MHz, DMSO) δ ppm 8.06 (s, 1H), 7.56 (s, 1H), 6.57 (s, br, 2H), 5.29 (pent, 1H), 5.06 (5, 1H), 4.84 (t, 1H), 3.27 (d, 2H), 2.58-2.50 (m, 2H), 2.26-2.19 (m, 2H).

### Intermediate S: (R,S)-7-(cis-3-Azidomethyl-cyclobutyl)-5-(2-phenyl-chroman-7-yl)-7H-pyrrolo[2,3-d]pyrimidin-4-ylamine

Toluene-4-sulphonic acid cis-3-[4-amino-5-(2-phenyl-chroman-7-yl)-pyrrolo[2,3-d]pyrimidin-7-yl]-cyclobutylmethyl ester (800 mg, 1.1 mmol) was dissolved in DMF (6 mL) and treated with sodium azide (279 mg, 4.3 mmol) at room temperature. The reaction was then stirred at 65° C. for 1 h. After cooling to room temperature the reaction mixture was submitted to an aqueous workup. The organic layers were dried and concentrated under reduced pressure to give the crude title compound which was used in the following reactions without further purification. ¹H NMR (400 MHz, CDCl₃) δ ppm 2.11-2.14 (m, 1H), 2.23-2.38 (m, 2H), 2.43-2.53 (m, 1H), 2.65-2.76 (m, 2H) 2.99-3.10 (m, 2H), 3.46 (d, 2H), 5.12 (d, 1H), 5.21 (quin, 1H), 5.29 (bs, 2H), 7.01 (d, 1H) 7.06 (s, 1H) 7.18 (d, 1H) 7.38-7.48 (m, 5H) 8.02 (s, 1H) 8.30 (s, 1H).

### Intermediate T: Toluene-4-sulphonic acid cis-3-[4-amino-5-(2-phenyl-chroman-7-yl)-pyrrolo[2,3-d]pyrimidin-7-yl]-cyclobutylmethyl ester

(R,S)-{cis-3-[4-Amino-5-(2-phenyl-chroman-7-yl)-pyrrolo[2,3-d]pyrimidin-7-yl]-cyclobutyl}-methanol (950 mg, 2.2 mmol) was dissolved in pyridine (10 mL) and the mixture cooled to −25° C. At this temperature p-toluenesulfonyl chloride (1.6 g, 8.9 mmol) was added in small portions over 30 min. Stirring at −25° C. was continued for 12 hours and the cooled reaction mixture quenched by addition of cold water and DCM. The organic layer was separated and repeatedly washed with cold aqueous 1N sulfuric acid solution. The organic layer was then dried and concentrated under reduced pressure to give the title compound as a yellow foam. HPLC/MS (Method Z) t_{R} 1.31 minute, M+H 489.3 (90%). HPLC_{A}: t_{R} 5.02 minutes.

### Intermediate U: 2-(Tetrahydro-furan-2-yl)-7-(4,4,5,5-tetramethyl-[1,3,2]dioxaborolan-2-yl)-chroman

A mixture of 7-bromo-2-(tetrahydro-furan-2-yl)-chroman (Step U.1, 760 mg, 2.68 mmol), bis(pinacolato)diboron (1.57 g, 6.17 mmol), dicyclohexyl(2′,6′-dimethoxybiphenyl-2-yl)phosphine (132 mg, 0.32 mmol), potassium phosphate (2.85 g, 13.42 mmol), palladium(II) acetate (36 mg, 0.161 mmol) and 1,4-doxane were heated under an argon atmosphere for 30 minutes at 100° C. The cooled reaction mixture was filtered through Hyflo, washing with 1,4-dioxane and evaporated. Purification of the residue by flash column chromatography, eluting with a gradient of methanol in DCM, gave the title compound as a pale yellow oil.

### Step U.1: 7-Bromo-2-(tetrahydro-furan-2-yl)-chroman

A mixture of 7-bromo-2-furan-2-yl-chroman-4-ol (Step U.2, 2.46 g, 8.34 mmol), platinum(IV) oxide (0.19 g, 0.83 mmol) and acetic acid (60 ml) was stirred under an atmosphere of hydrogen at room temperature until 3 equivalents of hydrogen gas were consumed (25.01 mmol). The reaction mixture was filtered through Hyflo, washed with methanol and evaporated. Purification of the residue by flash column chromatography, eluting with a gradient of methanol in DCM, gave the title compound as a colourless oil.

### Step U.2: 7-Bromo-2-furan-2-yl-chroman-4-ol

A solution of borane THF complex (1 M, 18 ml, 18.0 mmol) was added to a solution of 7-bromo-2-furan-2-yl-chroman-4-one (Step U.3, 3.21 g, 10.9 mmol) in THF (120 ml) cooled at 0° C. The reaction mixture was allowed to warm to room temperature over 15 minutes, then recooled to 0° C. and quenched by the slow addition of water. The mixture was extracted with ethyl acetate, the combined organic layers were washed with brine, dried over sodium sulphate and evaporated. Trituration of the residue with methanol gave the title compound as a white solid. ¹H NMR (400 MHz, DMSO) δ ppm 2.04-2.38 (m, 1H), 4.87 (br. s., 1H), 5.33 (d, J=10.9 Hz, 1H), 5.66 (d, J=6.6 Hz, 1H), 6.43-6.52 (m, 1H), 6.55 (d, J=3.1 Hz, 1H), 6.94 (d, J=2.0 Hz, 1H), 7.08 (dd, J=8.4, 1.8 Hz, 1H), 7.36 (d, J=8.2 Hz, 1H), 7.69 (s, 1 H).

### Step U.3: 7-Bromo-2-furan-2-yl-chroman-4-one

A mixture of (E)-1-(4-Bromo-phenyl)-3-furan-2-yl-propenone (Step U.4, 24.95 g, 85 mmol) and acetic acid were refluxed for 18 hours. The cooled reaction mixture was evaporated, the residue partitioned between water and DCM, the DCM layers dried over sodium sulphate and evaporated. Purification of the residue by flash column chromatography, eluting with a gradient of hexane and ethyl aetate, gave the title compound as pale-yellow solid. ¹H NMR (400 MHz, CDCl₃) δ ppm 2.95-3.03 (m, 1H), 3.19-3.29 (m, 1H), 5.52-5.58 (m, 1H), 6.37-6.42 (m, 1H), 6.42-6.47 (m, 1H), 7.16-7.20 (m, 1H), 7.21-7.24 (m, 1H), 7.45-7.49 (m, 1H), 7.75-7.80 (m, 1H).

### Step U.4: (E)-1-(4-Bromo-phenyl)-3-furan-2-yl-propenone

Potassium hydroxide (10.44 g, 186 mmol) was added to a solution of 4-bromo-2-hydroxacetophenone (20 g, 93 mmol) and furan-2-carbaldehyde (10.443 g, 109 mmol) in ethanol (200 ml) and the mixture stirred for 3.5 hours at room temperature. The volume was then reduced under vacuum and the residue partitioned between ditethyl ether and 1N aqueous HCl, the combined organic layers dried over sodium sulphate and evaporated. Trituration of the residue with hexanes gave the title compound as a yellow solid. ¹H NMR (400 MHz, CDCl₃) δ ppm 6.55 (dd, J=3.3, 1.8 Hz, 1H), 6.79 (d, J=3.5 Hz, 1H), 7.07 (dd, J=8.6, 1.6 Hz, 1H), 7.21 (d, J=2.0 Hz, 1H), 7.47 (d, J=14.8 Hz, 1H), 7.57 (s, 1H), 7.69 (d, J=14.8 Hz, 1H), 7.76 (d, J=8.6 Hz, 1H), 13.03 (s, 1H).

### Intermediates V and W: (S)—(S)-2-(Tetrahydro-furan-2-yl)-chroman-7-ol and (S)—(R)-2-(tetrahydro-furan-2-yl)-chroman-7-ol

A suspension of palladium on carbon (10%, 10 mg) in a solution of (S)-2-furan-2-yl-chroman-7-ol (Step V.1, 150 mg, 0.694 mmol) in acetic acid (10 ml) was stirred for 29 hours under an atmosphere of hydrogen, to take up 2 equivalents of hydrogen. The reaction mixture was then filtered and evaporated. Purification by normal phase chromatography, eluting with a gradient of methanol in DCM, was followed by reversed phase chromatography (Method R). Fractions containing each of the 2 diastereoisomers were separately partitioned between DCM and aqueous sodium hydrogen carbonate solution, the organic layers dried over sodium sulphate and evaporated to give the title compounds. The first eluting diastereoisomer by reversed phase HPLC (Intermediate V) was shown to be the like-stereoisomer by single-crystal X-ray structural analysis.

Intermediate V (S,S)-isomer as a yellow solid. HPLC/MS (Method Z) t_{R} 0.88 minutes, M+H 221.4 (100%). ¹H NMR (400 MHz, MeOD) δ ppm 6.83 (d, 1H), 6.28 (dd, 1H), 6.25 (d, 1H), 4.03-3.95 (m, 1H), 3.92-3.81 (m, 3H), 2.80-2.71 (m, 1H), 2.71-2.63 (m, 1H), 2.10-1.89 (m, 4H), 1.88-1.71 (m, 2H).

Intermediate W (S,R)-isomer as a yellow glass. HPLC/MS (Method Z) t_{R} 0.91 minutes, M+H 221.4 (100%). ¹H NMR (400 MHz, MeOD) δ ppm 6.81 (d, 1H), 6.27 (dd, 1H), 6.17 (d, 1H), 3.98-3.92 (m, 1H), 3.92-3.80 (m, 2H), 3.80-3.73 (m, 1H), 2.75-2.61 (m, 2H), 2.11-2.00 (m, 2H), 2.00-1.86 (m, 3H), 1.68-1.59 (m, 1H).

### Step V.1: (S)-2-Furan-2-yl-chroman-7-ol

A solution of tertabutylammonium fluoride (1 M, 3.1 ml, 3.10 mmol) was added to a solution of ((S)-2-furan-2-yl-chroman-7-yloxy)-triisopropyl-silane (Step V.2, 550 mg, 1.03 mmol) in THF (10 ml). After stirring for 45 minutes at room temperature the reaction mixture was partitioned between water and ethyl acetate, extracted 2× with ethyl acetate, the combined organic layers washed with brine, dried over sodium sulphate and evaporated. Purification by normal phase chromatography, eluting with a gradient of methanol in DCM, gave the title compound. HPLC/MS (Method Z) t_{R} 0.99 minutes, M+H 217.2 (60%).

### Step V.2: ((S)-2-Furan-2-yl-chroman-7-yloxy)-triisopropyl-silane

A solution of nBuLi in hexanes (2.5 M, 1.05 ml, 2.61 mmol) was added dropwise to a solution of [4-bromo-3-((S)-3-chloro-1-furan-2-yl-propoxy)-phenoxy]-triisopropyl-silane (850 mg, 1.74 mmol) in THF (5 ml) cooled to −78° C. The reaction mixture was stirred at −78° C. for 10 minutes, then warmed to 0° C. and stirred for a further 30 minutes before quenching by the addition of aqueous ammonium chloride solution. Extraction with DCM, drying the combined organic layers over sodium sulphate and evaporation gave a yellow oil which was purified by normal phase chromatography, eluting with a gradient of ethyl acetate in hexane, to give the title compound. ¹H NMR (400 MHz, CDCl₃) δ ppm 7.44 (d, 1H), 6.87 (d, 1H), 6.94-6.90 (m, 2H), 6.88-6.85 (m, 2H), 5.06 (dd, 1H), 2.92-2.82 (m, 1H), 2.77-2.71 (m, 1H), 2.29-2.18 (m, 2H), 1.12-1.04 (m, 18H), 0.96 (septet, 3H).

### Step V.3: [4-Bromo-3-((S)-3-chloro-1-furan-2-yl-propoxy)-phenoxy]trisopropyl-silane

Diisopropyl azodicarboxylate (1.15 ml, 5.91 mmol) was added to a mixture of (R)-3-chloro-furan-2-yl-propan-1-ol (Step V.4, 1.05 g, 5.91 mmol), 2-bromo-5-triisopropylsilanyloxy-phenol (step V.5, 1.7 g, 4.92 mmol), triphenylphosphine (1.36 g, 5.17 mmol) and THF (30 ml) at room temperature. After stirring 16 hours at room temperature the reaction mixture was partitioned between water and ethyl acetate, the combined organic layers washed with saturated brine, dried over sodium sulphate and evaporated. The residue was purified by normal phase chromatography, eluting with a gradient of ethyl acetate in hexanes, to give the title compound as a colourless oil. ¹H NMR (400 MHz, CDCl₃) δ ppm 7.37 (d, 1H), 7.30 (d, 1H), 6.48 (d, 1H), 6.41 (dd, 1H), 6.32 (d, 1H), 6.28 (d, 1H), 5.37-5.32 (m, 1H), 3.94-3.58 (m, 1H), 3.72-3.64 (m, 1H), 2.71-2.2.66 (m, 1H), 2.42-2.33 (m, 1H), 1.11 (septet, 3H), 1.07-1.01 (m, 18H).

### Step V.4: (R)-3-Chloro-furan-2-yl-propan-1-ol

Borane-THF complex (1 M, 37.8 ml, 37.8 mmol) was added to a solution of 3-chloro-1-furan-2-yl-propan-1-one (Step V.6, 10 g, 63.1 mmol) in THF (60 ml) cooled at 0° C. After 30 minutes at 0° C., the reaction mixture was partitioned between water and DCM, the combined organic layers dried over sodium sulphate and evaporated. The residue was purified by normal phase chromatography, eluting with a gradient of methanol in DCM, to give the title compound as a pale-brown oil. ¹H NMR (400 MHz, CDCl₃) δ ppm 7.48 (d, 1H), 6.84 (d, 1H), 6.77 (d, 1H), 4.98-4.92 (m, 1H), 3.78-3.72 (m, 1H), 3.71-3.56 (m, 1H), 2.38-2.19 (m, 2H).

### Step V.5: 2-Bromo-5-triisopropylsilanyloxy-phenol

To the stirred solution of 4-bromo-benzene-1,3-diol (71 g, 0.376 mol) and DMF (1 ml) was subsequently added imidazole (25.6 g, 0.376 mol) and triisopropylsilyl chloride (80 ml, 0.376 mol) at 0° C. The reaction mixture was stirred for 3 days at room temperature and then partitioned between HCl 0.5M and tert-butylmethyl ether. The combined organic layers were washed with water and brine, dried over sodium sulphate and evaporated. The residue was purified by normal phase chromatography, eluting with a gradient of ethyl actetate in n-heptane, to give the title compound as slightly yellow oil. TLC (ethyl actetate n-heptane 1:10) Rf=0.33. ¹H NMR (600 MHz, DMSO-d₆) δ ppm 10.16 (s, 1H), 7.29 (d, 1H), 6.53 (s, 1H), 6.28 (d, 1H), 1.22 (m, 3H), 1.04 (m, 18H).

### Step V.6: 3-Chloro-1-furan-2-yl-propan-1-one

A solution of furan (5 g, 72.7 mmol) in 1,2-dichloroethane (5 ml) was added dropwise over 30 minutes to a suspension of aluminium chloride (10.7 g, 80.0 mmol) in 1,2-dichloroethane (35 ml) cooled at 0° C. After stirring for a further 2 hours at 0° C. the reaction mixture was poured into a mixture of ice and water and filtered. The organic layer was washed with saturated aqueous sodium hydrogencarbonate solution, dried over sodium sulphate and evaporated to give the title compound as a brown oil. ¹H NMR (400 MHz, CDCl₃) δ ppm 7.61 (d, 1H), 7.24 (d, 1H), 6.57 (dd, 1H), 3.89 (t, 2H), 3.32 (t, 2H).

### Intermediate X: (S)-2-Phenyl-7-(4,4,5,5-tetramethyl-[1,3,2]dioxaborolan-2-yl)-chroman

A mixture of (S)-7-bromo-2-phenyl-chroman (Intermediate Z, 1.68 g, 5.81 mmol), bis(pinacolato)diboron (2.95 g, 11.62 mmol), potassium acetate (1.71 g, 17.43 mmol), [1,1-bis(diphenylphosphino)ferrocene]dichloropalladium(II)-DCM complex (0.47 g, 0.58 mmol) and DMSO (18 ml) were heated under an argon atmosphere for 1 hour at 100° C. The cooled reaction mixture was partitioned between saturated sodium bicarbonate solution and ethyl acetate, the combined organic layers washed with brine, dried over sodium sulphate and evaporated. The residue was purified by normal phase chromatography, eluting with a gradient of ethyl acetate in hexanes, to give the title compound as a white solid. ¹H NMR (400 MHz, CDCl₃) δ ppm 7.44-7.25 (m, 7H), 7.09 (d, 1H), 5.08 (dd, 1H), 3.04-2.94 (m, 1H), 2.84-2.74 (m, 1H), 2.28-2.19 (m, 1H), 2.10-1.99 (m, 1H), 1.33 (s, 12H).

### Intermediate Y: (R)-2-Phenyl-7-(4,4,5,5-tetramethyl-[1,3,2]dioxaborolan-2-yl)-chroman

The title compound is prepared in an analagous manner to Intermediate X using (R)-7-bromo-2-phenyl-chroman (Intermediate AA) in place of (S)-7-bromo-2-phenyl-chroman (Intermediate Z).

Alternatively, Intermediate Y (R)-2-phenyl-7-(4,4,5,5-tetramethyl-[1,3,2]dioxaborolan-2-yl)-chroman can be prepard in the following manner.

A mixture of trifluoro-methanesulfonic acid (R)-2-phenyl-chroman-7-yl ester (Intermediate Y.1, 92 mg, 0.22 mmol), bis(pinacolato)diboron (79 mg, 0.31 mmol), potassium acetate (130 mg, 0.133 mmol), [1,1-bis(diphenylphosphino)ferrocene]dichloropalladium(II)-DCM complex (24.2 mg, 0.033 mmol) and dioxane (1 ml) were heated under an argon atmosphere for 3 hours at 90° C. The cooled reaction mixture was partitioned between ammonium chloride solution and ethyl acetate, the combined organic layers washed with brine, dried over sodium sulphate and evaporated. The residue was purified by flash chromatography, eluting with ethyl acetate in hexanes (1:4), to give the title compound as a white solid. ¹H NMR (600 MHz, DMSO-d₆) δ ppm 7.43-7.39 (m, 4H), 7.32 (t, 1H), 7.14 (d, 1H), 7.12 (d, 1H), 7.09 (s, 1H), 5.14 (d, 1H), 2.96 (m, 1H), 2.73-2.70 (m, 1H), 2.21-2.15 (m, 1H), 1.95 (m, 1H), 1.27 (s, 12H).

### Step Y.1: Trifluoro-methanesulfonic acid (R)-2-phenyl-chroman-7-yl ester

To the stirred solution of (R)-2-phenyl-chroman-7-ol (Intermediate Y.2, 72 mg, 0.318 mmol), triethylamine (0.053 ml, 0.382 mmol) and dichloromethane (1 ml) was added dropwise a solution of N-phenyl-bis(trifluoromethane sulfonamide) (0.125 g, 0.350 mmol) and dichloromethane (0.5 ml) at 0° C. The reaction mixture was stirred for 3 hours at 0° C. and then partioned between saturated sodium bicarbonate solution and tert-butylmethyl ether. The combined organic layers were washed with brine, dried over sodium sulphate and evaporated. The residue was purified by flash chromatography, eluting with ethyl acetate in hexanes (1:3), to give the title compound as a white solid. HPLC/MS (Method Z) t_{R} 1.39 minutes, M−H 357.1 (87%).

### Step Y.2: (R)-2-Phenyl-chroman-7-ol

To the stirred solution of triisopropyl-((R)-2-phenyl-chroman-7-yloxy)-silane (Intermediate Y.3, 0.65 g, 0.68 mmol) and THF (10 ml) was added TBAF in THF (1M, 2.04 ml, 2.04 mmol) at 0° C. The reaction mixture was stirred for 1 hour at 0° C. and then partitioned between water and ethyl acetate. The combined organic layers were washed with water and brine, dried over sodium sulphate and evaporated. The residue was purified by reversed phase preparative chromatography (Method R) to give the title compound as a oil. HPLC/MS (Method Z) t_{R} 1.04 minutes, M+H 227.1. ¹H NMR (600 MHz, DMSO-d₆) δ ppm 9.17 (s, 1H), 7.41-7.39 (m, 4H), 7.32 (m, 1H), 6.86 (d, 1H), 6.28 (d, 1H), 6.21 (s, 1H), 5.04 (d, 1H), 2.81 (m, 1H), 2.59-2.57 (m, 1H), 2.09 (m, 1H), 1.94 (m, 1H).

### Step Y.3: Triisopropyl-((R)-2-phenyl-chroman-7-yloxy)-silane

To the stirred solution of [4-bromo-3-((R)-3-chloro-1-phenyl-propoxy)-phenoxy]-triisopropyl-silane (Intermediate Y.4, 1.20 g, 2.41 mmol) and THF (24 ml) was added dropwise n-butyl lithium in n-hexane (1.6M, 1.58 ml, 2.53 mmol) at −50° C. The reaction mixture was stirred at −50° C. for 10 minutes, then warmed to room temperature and stirred for a further 3 hours before quenching by the addition of brine. Extraction with ethyl acetate, drying the combined organic layers over sodium sulphate and evaporation gave the crude title compound as colourless oil which was used in the next step without purification.

### Step Y.4: [4-Bromo-3-((R)-3-chloro-1-phenyl-propoxy)-phenoxy]triisopropyl-silane

To the stirred solution of 2-bromo-5-triisopropylsilanyloxy-phenol (Intermediate V.5, 1.045 g, 3.0 mmol), (S)-3-chloro-1-phenylpropan-1ol and THF (30 ml) were subsequently added triphenylphosphine (1.57 g, 6.0 mmol) and diisopropyl azodicarboxylate (1.24 ml, 6.0 mmol). The reaction mixture was stirred for 1 hour at room temperature and then partitioned between brine and ethyl acetate. The combined organic layers were dried over sodium sulphate and evaporated. The residue was purified by flash chromatography, eluting with ethyl acetate in hexanes (1:30), to give the title compound as beige oil. ¹H NMR (400 MHz, DMSO-d6) δ ppm 7.25-7.40 (m, 6H), 6.34 (d, 1H), 6.23 (s, 1H), 5.40 (m, 1H), 3.85 (m, 1H), 3.72 (m, 1H), 2.39 (m, 1H), 2.19 (m, 1H), 1.10-0.80 (m, 21H).

### Intermediates Z and AA: (S)-7-Bromo-2-phenyl-chroman and (R)-7-bromo-2-phenyl-chroman

The racemic mixture of 7-bromo-2-phenyl-chroman (Step Z.1) was separated by preparative chiral chromatography to give the individual enantiomers.

Column: CHIRALPAK-AD prep. (20 um) 5×50 cm
Eluent (Mobile-phase): 70% n-Heptane 99% HPLC (Art. Nr. 08071677 Biosolve Chemie Brunschwig AG), 30% Ethanol (Art. Nr. 02855 Sigma-Aldrich GmbH)
Flow: 30 ml/min

### Detection: UV=210 nm

### Injection: 1.0 g in 10 ml EtOH/Mobile-phase 50:50

First eluting (S)-enantiomer
Second eluting (R)-enantiomer

### Step Z.1: 7-bromo-2-phenyl-chroman

n-Butylsilane (8.9 ml, 68.5 mmol) was added dropwise over 15 minutes to a solution of 7-bromo-2-phenyl-chroman-4-ol (Step Z.2, 20.9 g, 68.5 mmol) and tris(pentafluorophenyl)-borane (1.81 g, 3.42 mmol) in DCM (420 ml) cooled at 0° C., caution: gas evolution! The reaction mixture was stirred for 3 hours at 0° C., aqueous sodium bicarbonate solution added, extracted with diethyl ether, the combined organic layers washed with water and then brine, dried over sodium sulphate and evaporated. The residue was purified by normal phase chromatography, eluting with a gradient of tert-butylmethyl ether in hexanes, to give the title compound as a viscous oil. ¹H NMR (400 MHz, CDCl₃) δ ppm 7.34-7.24 (m, 5H), 7.02 (dd, 1H), 6.93 (dd, 1H), 6.67 (d, 1H), 4.98 (dd, 1H), 2.88-2.78 (m, 1H), 2.70-2.63 (m, 1H), 2.18-2.11 (m, 1H), 2.07-1.92 (m, 1H).

Alternatively, Step Z.1 7-bromo-2-phenyl-chroman can be prepared in the following manner. A suspension of platinum(IV) oxide (65 mg, 0.29 mmol) in a solution of 7-bromo-2-phenyl-chromen-4-one (Intermediate H, 325 mg, 1.13 mmol) in THF (10 ml) was stirred under an atmosphere of hydrogen for 1.5 hours (1 equiv. hydrogen taken up, 1.13 mmol). The reaction mixture was then filtered and evaporated. The residue was purified by normal phase chromatography, eluting with a gradient of ethyl acetate in hexane, to give the title compound. ¹H NMR (400 MHz, CDCl₃) δ ppm 2.01-2.13 (m, 1H), 2.18-2.27, (m, 1H), 2.69-2.79 (m, 1H), 2.86-2.97 (m, 1H), 5.07 (dd, 1H), 6.92-6.97 (m, 1H), 6.97-7.02 (m, 1H), 7.09 (d, 1H), 7.32-7.36 (m, 1H), 7.39 (d, 3H).

### Step Z. 2: 7-Bromo-2-phenyl-chroman-4-ol

Sodium borohydride (2.61 g, 69.0 mmol) was added portionwise to a solution of 7-bromo-2-phenyl-chroman-4-one (Step Z.3, 20.92 g, 69.0 mmol) in methanol (800 ml) at room temper-ature. After stirring for 2 hours at room temperature the reaction mixture was partitioned between saturated sodium bicarbonate solution and ethyl acetate, the organic layer washed with water and brine, dried over sodium sulphate and evaporated to give the title compound. ¹H NMR (400 MHz, CDCl₃) δ ppm 7.49-7.37 (m, 6H), 7.16-7.09 (m, 2H), 5.22 (dd, 1H), 5.11-5.05 (m, 1H), 2.61-2.52 (m, 1H), 2.21-2.11 (m, 1H), 1.62 (s, br, 1H).

### Step Z.3: 7-Bromo-2-phenyl-chroman-4-one

(E)-1-(4-Bromo-2-hydroxy-phenyl)-3-phenyl-propenone (Intermediate I, 10.79 g, 35.6 mmol) was added to acetic acid (130 ml) and stirred for 56 hours at room temperature. The reaction mixture was evaporated and partitioned between ethyl acetate and aqueous sodium bicarbonate solution, the combined organic layers washed with water and brine, dried over sodium sulphate and evaporated. The residue was purified by normal phase chromatography, eluting with a gradient of DCM in heptane, to give the title compound. ¹H NMR (400 MHz, CDCl₃) δ ppm 7.82 (d, 1H), 7.53-7.39 (m, 5H), 7.32 (d, 1H), 7.23 (d, 1H), 5.53 (dd, 1H), 3.12 (dd, 1H), 2.93 (dd, 1H).

### Intermediate AB: 8-[cis-3-(4-Amino-5-bromo-pyrrolo[2,3-d]pyrimidin-7-yl)-cyclobutylmethyl]-8-aza-bicyclo[3.2.1]octan-(3-exo)-ol

The title compound was prepared in analogy to the procedure described for the preparation of Intermediate AC, but with the following modifications. Regarding the procedure of intermediate AC, the crude product was purified by silica gel column chromatography (DCM/MeOH/NH₃^{aq}, 89:10:1). In step AC.1, 1 eq of Br₂ was used. In step AC.2, the reaction mixture was stirred for 1 h after addition of 6N HCl. In step AC.3, the reaction time was 1 h and the intermediate prepared in step AB.1 was used. The title compound: ES-MS: 406/408 [M+H]⁺; R_{f}=0.14 (DCM/MeOH/NH₃^{aq}, 89:10:1).

### Step AB.1: [cis-3-((3-exo)-Hydroxy-8-aza-bicyclo[3.2.1]oct-8-ylmethyl)-cyclobutyl]-carbamic acid benzyl ester

Sodium triacetoxyborohydride (1.8 g, 8.6 mmol, 2 eq) was added to a mixture of (cis-3-formyl-cyclobutyl)-carbamic acid benzyl ester (Step O.5, 1.0 g, 4.3 mmol) and 8-aza-bicyclo[3.2.1]octan-(3-exo)-ol (Baeckvall, J. E.; Renko, Z. D.; Bystroem, S. E.; Tetrahedron Letters (1987), 28(36), 4199-4202) (0.91 g, 5.6 mmol, 1.3 eq) in DCM (20 mL), under an argon atmosphere. The resulting mixture was stirred for 2 h at rt, quenched by addition of NaHCO₃^{sat} and extracted with DCM. The combined organic layers were dried (Na₂SO₄), filtered and concentrated. The residue was purified by silica gel column chromatography (DCM/MeOH/NH₃^{aq}, 94:5:1) to afford 1.1 g of the title compound as a white solid: ES-MS: 345.3 [M+H]+; R_{f}=0.07 (DCM/MeOH/NH₃^{aq}, 94:5:1).

### Intermediate AC: 1-[cis-3-(4-Amino-5-bromo-pyrrolo[2,3-d]pyrimidin-7-yl)-cyclobutylmethyl]-piperidin-4-ol

A mixture of 1-[cis-3-(5-bromo-4-chloro-pyrrolo[2,3-d]pyrimidin-7-yl)-cyclobutylmethyl]-piperidin-4-ol (Step AC.1, 91 mg, 0.228 mmol), NH₄OH (30% NH₃ aqueous solution, 1 mL) and EtOH (1 mL) was stirred at 120° C. for 16 h in a sealed vessel, allowed to cool to rt and diluted in ethyl acetate/H₂O. The aqueous layer was extracted with DCM. The combined organic extracts were washed with H₂O and brine, dried (Na₂SO₄), filtered and concentrated. The crude material was purified by silica gel column chromatography (DCM/MeOH/NH₃^{aq}, 91.5:7.5:1) to afford 59 mg of the title compound as a white foam: ES-MS: 380/382 [M+H]⁺; R_{f} 0.06 (DCM/MeOH/NH₃^{aq}, 91.5:7.5:1).

### Step AC.1: 1-[cis-3-(5-Bromo-4-chloro-pyrrolo[2,3-d]pyrimidin-7-yl)-cyclobutylmethyl]-piperidin-4-ol

A mixture of the intermediate prepared in step AC.2 (185 mg, 0.577 mmol), bromine (0.036 mL, 0.692 mmol, 1.2 eq) and AcOH (1 mL) was stirred for 30 min at rt, concentrated, diluted with NaHCO₃^{sat}/DCM and extracted with DCM. The combined organic layers were dried (Na₂SO₄), filtered and concentrated. The residue was purified by silica gel column chromatography (DCM/MeOH/NH₃^{aq}, 94:5:1) to afford 96 mg of the title compound: ES-MS: 399/401 [M+H]⁺; R_{f}=0.19 (DCM/MeOH/NH₃^{aq}, 94:5:1).

### Step AC.2: 1-[cis-3-(4-Chloro-pyrrolo[2,3-d]pyrimidin-7-yl)-cyclobutylmethyl]-piperidin-4-ol

A mixture of (4,6-dichloro-pyrimidin-5-yl)-acetadehyde (612 mg, 3.20 mmol), the intermediate prepared in step AC.3 (590 mg, 3.20 mmol) and DIEA (559 4, 3.20 mmol) in EtOH (10 mL) was stirred for 2 h at reflux, allowed to cool at rt, concentrated, diluted with a 6N aqueous solution of HCl, stirred for 10 min, basified by addition of NaHCO₃ and extracted with DCM. The residue was purified by silica gel column chromatography (DCM/MeOH/NH₃^{aq}, 94:5:1) to afford 702 mg of the title compound: ES-MS: 321 [M+H]⁺; R_{f}=0.14 (DCM/MeOH/NH₃^{aq}, 94:5:1).

### Step AC.3: 1-(cis-3-Amino-cyclobutylmethyl)-piperidin-4-ol

A mixture of the intermediate prepared in step AC.4 (1.03 g, 3.2 mmol) and palladium on carbon (200 mg) in MeOH (20 mL) was stirred for 16 h at rt and under a hydrogen atmosphere. The reaction mixture was filtered through a pad of celite and concentrated to afford 595 mg of the title compounds as a white solid: API-ES: 185 [M+H]⁺.

### Step AC.4: [cis-3-(4-Hydroxy-piperidin-1-ylmethyl)-cyclobutyl]-carbamic acid benzyl ester

Sodium triacetoxyborohydride (1 g, 4.7 mmol, 1.1 eq) was added to a mixture of (cis-3-formyl-cyclobutyl)-carbamic acid benzyl ester (Step O.5, 1.0 g, 4.3 mmol) and 4-hydroxypiperidine (0.87 g, 8.6 mmol, 2 eq) in DCM (20 mL), under an argon atmosphere. The resulting mixture was stirred for 10 min at rt, quenched by addition of NaHCO₃^{sat} and extracted with DCM. The combined organic layers were washed with H₂O, brine, dried (Na₂SO₄), filtered and concentrated. The residue was purified by silica gel column chromatography (DCM/MeOH/NH₃^{aq}, 91.5:7.5:1) to afford 1 g of the title compound as a white solid: ES-MS: 319.2 [M+H]+; R_{f}=0.15 (DCM/MeOH/NH₃^{aq}, 91.5:7.5:1).

### Intermediate AD: 1-{4-[cis-3-(4-Amino-5-bromo-pyrrolo[2,3-d]pyrimidin-7-yl)-cyclobutyl]-piperazin-1-yl}-ethanone

The title compound was prepared in analogy to the procedure described in Step AC.1 but with the following modifications. The intermediate prepared in Step AD.1 was used. The reaction mixture was stirred for 18 h at 120° C., concentrated and the residue was purified by silica gel column chromatography (DCM/MeOH/NH₃^{aq}, 98:1:1 then 95:4:1) to afford the title compound: ES-MS: 393/395 [M+H]⁺; R_{f}=0.44 (DCM/MeOH, 9:1).

### Step AD.1: 1-{4-[cis-3-(5-Bromo-4-chloro-pyrrolo[2,3-d]pyrimidin-7-yl)-cyclobutyl]-piperazin-1-yl}-ethanone

A mixture of the intermediate prepared in step AD.2 (960 mg, 2.88 mmol), bromine (0.148 mL, 2.88 mmol) and AcOH (6 mL) was stirred for 30 min at rt, quenched by addition of NaH-CO₃^{sat} and extracted with DCM. The combined organic layers were washed with NaHCO₃^{sat}, dried (Na₂SO₄), filtered and concentrated. The residue was purified by silica gel column chromatography (DCM/MeOH/NH₃^{aq}, 98:1:1 then 97:2:1) to afford 114 mg of the pure (cis isomer) title compound and 735 mg of an isomeric (cis/trans, 3:7) mixture (step BZ.1). The title compound (cis isomer): ES-MS: 412.1/414.1 [M+H]⁺; R_{f}=0.43 (DCM/MeOH, 9:1).

### Step AD.2: 1-{4-[3-(4-Chloro-pyrrolo[2,3-d]pyrimidin-7-yl)-cyclobutyl]-piperazin-1-yl}-ethanone

The title compound was prepared in analogy to the procedure described in step AC.2 but with the following modifications. The intermediate prepared in step AD.3 and 2.2 eq of DIEA were used. The reaction mixture was stirred for 18 h at 80° C., concentrated, diluted with a 6N aqueous solution of HCl, stirred for 10 min, basified by addition of NaHCO₃ and extracted with DCM. The residue was purified by silica gel column chromatography (DCM/MeOH/NH₃^{aq}, 99:0:1 then 97:2:1) to afford the title compound: ES-MS: 334.2 [M+H]⁺; R_{f}=0.47 (DCM/MeOH, 9:1).

### Step AD.3: 1-[4-(3-Amino-cyclobutyl)-piperazin-1-yl]-ethanone

A mixture of the intermediate prepared in step AD.4 (1.49 g, 5.0 mmol) and TFA (3.9 mL, 50 mmol, 10 eq) in DCM (4 mL) was stirred for 1 h at rt, concentrated, diluted with a 7N solution of NH₃ in MeOH and concentrated. The residue was purified by silica gel column chromatography (DCM/MeOH/NH₃^{aq}, 96:3:1 then 92:7:1) to afford 843 mg of the title compound as a white solid: ES-MS: 198.2 [M+H]⁺; R_{f}=0.02 (DCM/MeOH, 9:1).

### Step AD.4: [3-(4-Acetyl-piperazin-1-yl)cyclobutyl]-carbamic acid tert-butyl ester

A mixture of (3-oxo-cyclobutyl)-carbamic acid tert-butyl ester (1 g, 5.40 mmol, purchased from PrincetonBio), 1-acetyl-piperazine (0.830 g, 6.48 mmol, 1.2 eq) and sodium triacetoxyborohydride (3.43 g, 16.20 mmol, 3 eq) in DCM (10 mL) was stirred for 1 h at rt, quenched by addition of NaHCO₃^{sat} and extracted with DCM. The combined organic layers were washed with NaHCO₃^{sat}, dried (Na₂SO₄), filtered and concentrated. The residue was purified by silica gel column chromatography (DCM/MeOH/NH₃^{aq}, 98:1:1 then 95:4:1) to afford 1.49 g of the title compound as a white solid: ES-MS: 298.3 [M+H]+; R_{f}=0.34 (DCM/MeOH, 9:1).

### Intermediate AE: (E)-1-Aminomethyl-3-[4-amino-5-(2-phenyl-chroman-7-yl)-pyrrolo[2,3-d]pyrimidin-7-yl]-cyclobutanol

(E)-3-[4-Amino-5-(2-phenyl-chroman-7-yl)-pyrrolo[2,3-d]pyrimidin-7-yl]-1-azidomethylcyclobutanol (Step AE.1, 0.52 g, 1.06 mmol) and triphenylphosphine (0.429 g, 1.59 mmol)) were dissolved in THF (7.5 mL), MeOH (7.5 mL), H₂O (1.9 mL), and aqueous ammonia (25%, 0.66 mL) and stirred under Ar at RT for 16 h. After adding H₂O (6 mL) and evaporating the organic solvents, the reaction mixture was extracted with DCM (10 mL, 3×). The combined organic phases were washed with brine (2 mL), dried (Na₂SO₄), and concentrated under reduced pressure. The resulting residue was taken up in DCM (3 mL), Isolute was added and the solvent was evaporated. The Isolute absorbed material was transferred on a Redi-Sept silica gel column (40 g) and eluted by using a Flash-Master2.0 chromatographer (DCM->DCM/MeOH with 10% aqueous ammonia (25%)=4:1 in 35 min, 40 mL/min) yielding the title compound as white solid. HPLC (Method B) t^{Ret}=2.36 min. HPLC/MS t_{R}=0.79 min, M+H=442.3. ¹H NMR (600 MHz, DMSO-d₆) δ ppm 8.13 (s, 1H), 7.58 (s, 1H), 7.46 (d, 2H), 7.40 (t, 2H), 7.34 (t, 1H), 7.21 (d, 1H), 6.97 (d, 1H), 6.96 (s, 1H), 6.15 (s/b, 2H), 5.32 (quintet, 1H), 5.21 (d, 1H), 5.09 8 (s/broad, 1H), 3.00/2.55 (m/m, 1H/1H), 2.61 (s, 2H), 2.55/2.30 (t/t, 2H/2H), 2.22/2.02 (m/m, 1H/1H).

### Step AE.1: (E)-3-[4-Amino-5-(2-phenyl-chroman-7-yl)-pyrrolo[2,3-d]pyrimidin-7-yl]-1-azidomethylcyclobutanol

Toluene-4-sulfonic acid 3-[4-amino-5-(2-phenyl-chroman-7-yl)-pyrrolo[2,3-d]pyrimidin-7-yl]-1-hydroxy-cyclobutylmethyl ester (Step AE.2, 665 mg, 1.088 mmol) and NaN₃ (286 mg, 4.35 mmol) dissolved in DMF (5 mL, dried over molecular sieve) were stirred at 65° C. for 3 h under Ar. After adding H₂O (10 mL), the reaction mixture was extracted with AcOEt (20 mL, 3 x). The combined organic phases were washed with brine (5 mL), dried (MgSO₄), and the solvent was evaporated under reduced pressure to give the title compound as white solid. HPLC (Method B) t^{Ret}=2.78 min. HPLC/MS t_{R}=1.16 min, M+H=468.3. ¹H NMR (600 MHz, DMSO-d₆) δ ppm 8.13 (s, 1H), 7.63 (s, 1H), 7.46 (d, 2H), 7.40 (t, 2H), 7.34 (t, 1H), 7.21 (d, 1H), 6.97 (d, 1H), 6.96 (s, 1H), 6.15 (s/b, 2H), 5.69 (s, 1H), 5.43 (quintet, 1H), 5.21 (d, 1H), 3.44 (s, 1H), 3.00/2.55 (m/m, 1H/1H), 2.55/2.30 (t/t, 2H/2H), 2.22/2.02 (m/m, 1H/1H).

### Step AE.2: Toluene-4-sulfonic acid (E)-3-[4-amino-5-(2-phenyl-chroman-7-yl)-pyrrolo[2,3-d]pyrimidin-7-yl]-1-hydroxy-cyclobutylmethyl ester

[(E)-3-[4-Amino-5-(2-phenyl-chroman-7-yl)-pyrrolo[2,3-d]pyrimidin-7-yl]-1-hydroxymethyl-cyclobutanol (Examples 42 and 43, 1.63 g 3.68 mmol), dibutyltin oxide (1.01 g (4.05 mmol) dissolved in DCM/CHCl₃/MeOH (94 mL, 3:6:1) were stirred at 65° C. for 2 h under Ar. After evaporating the solvent and drying the resulting white residue under vacuum at 50° C. for 4 h, DCM (100 mL) and Ts—Cl (780 mg, 4.05 mmol) were added and the reaction mixture was stirred at RT for 90 min. After adding H₂O (0.13 mL, 7.37 mmol) the reaction mixture was vigorously stirred at RT for 30 min. After evaporating the solvent, the residue was taken up in DCM (6 mL) and filtered over a frit filled with silica gel (16 g, elution with AcOEt followed by elution of the product with DCM/MeOH/aqueous NH3 (9:1:01)) to give the title compound as white solid. HPLC (method B) t^{Ret}=2.93 min. HPLC/MS t_{R}=1.29 min, M+NH₃=597.3. ¹H NMR (600 MHz, DMSO-d₆) δ ppm 8.13 (5, 1H), 7.84 (d, 2H), 7.57 (s, 1H), 7.53 (d, 2H), 7.43 (d, 2H), 7.40 (1, 2H), 7.34 (t, 1H), 7.21 (d, 1H), 6.97 (d, 1H), 6.96 (s, 1H), 6.15 (s/b, 2H), 5.80 (s, 1H), 5.19 (d, 1H), 5.80 (quintet, 1H), 4.09 (s, 2H), 3.00/2.55 (m/m, 1H/1H), 2.39 (s, 3H), 2.55/2.30 (t/t, 2H/2H), 2.22/2.02 (m/m, 1H/1H).

### Intermediate AF: 4-[cis-3-(4-Amino-5-iodo-pyrrolo[2,3-d]pyrimidin-7-yl)-cyclobutylmethyl]-1-methyl-piperazin-2-one

A solution of 4-[cis-3-(4-chloro-5-iodo-pyrrolo[2,3-d]pyrimidin-7-yl)-cyclobutylmethyl]-1-methyl-piperazin-2-one (Step AF.1, 313 mg, 0.681 mmol) and aqueous ammonia (25%, 8.2 mL) in dioxan (4.1 mL) was stirred in a glass autoclave (10 mL, Büchi) at 100° C. for 16 h. After evaporation of the solvent, the resulting residue was purified by means of a Sepacore Control chromatographer (Büchi, Flawil, Switzerland) using RediSept silica gel column (12 g) (30 mL/min; DCM:10 min, DCM->DCM/MeOH/NH3 (90:5:0.5) in 30 min) yielding the title compound as white solid. HPLC (method B) t^{Ret}=1.724 min. HPLC/MS t_{R}=0.52 min, M+H=441.1. ¹H NMR (600 MHz, DMSO-d₆) δ ppm 8.16 (s, 1H), 7.74 (s, 1H), 6.15 (s/b, 2H), 5.00 (quintet, 1H), 3.26 (t, 2H), 2.95 (s, 2H), 2.78 (s, 3H), 2.65 (t, 2H), 2.56 (t, 2H), 2.50/2.15 (m/m, 2H/2H), 2.29 (m, 1H).

### Step AF.1: 4-[3-(4-Chloro-5-iodo-pyrrolo[2,3-d]pyrimidin-7-yl)-cyclobutyl methyl]-1-methyl-piperazin-2-one

Methylpiperazin-2-one HCl salt (290 mg, 1.2832 mmol), 3-(4-Chloro-5-iodo-pyrrolo[2,3-d]pyrimidin-7-yl)-cyclobutanecarbaldehyde (Step AF.2, 265 mg, 0.733 mmol), and DIPEA (1.306 mL, 7.33 mmol) were dissolved in 1,2-dichloroethane (32 mL) and stirred at RT for 45 min. After adding NaBH(OAc)₃ (409 mg, 1.832 mmol) the reaction mixture was stirred for 35 min at RT. Then, concentrated NaHCO3 solution (50 mL) was added and the reaction mixture was extracted with DCM (40 mL, 4×). The combined organic phases were washed with brine (10 mL), dried (Na₂SO₄), and the solvent was evaporated. the resulting residue was purified by means of a Sepacore Control chromatographer (Büchi, Flawil, Switzerland) using RediSept silica gel column (12 g) (30 mL/min; DCM:10 min, DCM->DCM/MeOH/NH3 (99.45:0.5:05) in 30 min) yielding the title compound as white solid. HPLC (Method B) t″=1.724 min. HPLC/MS t_{R}=0.52 min, M+H=441.1. ¹H NMR (600 MHz, DMSO-d₆) δ ppm 8.16 (s, 1H), 7.74 (s, 1H), 6.15 (s/b, 2H), 5.00 (quintet, 1H), 3.26 (t, 2H), 2.95 (s, 2H), 2.78 (s, 3H), 2.65 (t, 2H), 2.56 (t, 2H), 2.50/2.15 (m/m, 2H/2H), 2.29 (m, 1H).

### Step AF.2: cis-3-(4-Chloro-5-iodo-pyrrolo[2,3-d]pyrimidin-7-yl)-cyclobutanecarbaldehyde

To a stirred solution of (Z)-3-(4-chloro-5-iodo-pyrrolo[2,3-d]pyrimidin-7-yl)-cyclobutanecarboxylic acid methyl ester (prepared as described in WO2005097800, 2.77 g, 7.07 mmol) and DCM (40 mL) was added slowly DIBAL-H (1.7M in toluene) (5.41 mL, 9.20 mmol, 1.3 eq) at −78° C. Stirring was continued for 4 h at −78° C. The reaction mixture was quenched with sat. ammonium chlorid (5 mL). The reaction mixture was partitioned between HCl 1M and DCM. The combined organic layers were washed with brine, dried (Na₂SO₄), filtered and concentrated. The residue was purified by silica gel column chromatography (EtOAc-n hexane 1:1) to afford 2.01 g of the title compound as white solid: HPLC-MS: M+H=362.0 (Rt=1.13. ¹H NMR (400 MHz, DMSO-d₆) δ ppm 9.70 (s, 1H), 8.62 (s, 1H), 8.26 (s, 1H), 5.29 (m, 1H), 3.08 (m, 1H), 2.73 (m, 2H), 2.61 (m, 2H).

### Intermediate AG: 2-Ethoxymethyl-7-(4,4,5,5-tetramethyl-[1,3,2]dioxaborolan-2-yl)-chroman

A mixture of trifluoro-methanesulfonic acid 2-ethoxymethyl-chroman-7-yl ester (Step AG.1, 1.14 g, 3.35 mmol), bis-(pinacolato)-diboron (1.91 g, 4.69 mmol), potassium acetate (0.99 g, 10.1 mmol), [1,1-bis(diphenylphosphino)ferrocene]dichloropalladium(II)-DCM complex (0.41 g, 0.50 mmol) and dioxane were heated at 90° C. for 2.25 hours under an argon atmosphere. The cooled reaction mixture was taken up in ethyl acetate and washed with aqueous ammonium chloride and brine, dried over sodium sulphate and evaporated. The isolated material was purified by normal phase chromatography, eluting with a gradient of methanol in DCM to give the title compound as an orange oil.

### Step AG.1: Trifluoro-methanesulfonic acid 2-ethoxymethyl-chroman-7-yl ester

N-Phenyl-bis(trifluoromethanesulphonamide) (1.89 g, 5.28 mmol) was added to a mixture of 2-ethoxymethyl-chroman-7-ol (Step AG.2, 1.0 g, 4.80 mmol), triethylamine (0.80 ml, 5.76 mmol) and DCM (24 ml) at 0° C. After stirring for 3 hours at 0° C. aqueous NaHCO₃ was added, the organic layer dried over Na₂SO₄ and evaporated. The isolated material was purified by normal phase chromatography, eluting with a gradient of ethyl acetate in hexane to give the title compound as a clear colourless oil. ¹H NMR (400 MHz, CDCl₃) δ ppm 1.21-1.29 (m, 3H), 1.76-1.88 (m, 1H), 2.02-2.11 (m, 1H), 2.73-2.91 (m, 2H), 3.55-3.63 (m, 3H), 3.64-3.71 (m, 1H), 4.16-4.24 (m, 1H), 6.72-6.76 (m, 1H), 6.76-6.79 (m, 1H), 7.07 (d, J=8.6 Hz, 1H).

### Step AG.2: 2-Ethoxymethyl-chroman-7-ol

A suspension of PdIC (1 g, 10% ex Engelhard 4505) in a solution of 7-benzyloxy-2-ethoxymethyl-chroman (Step AG.3, 6.25 g, 19.9 mmol) in methanol was stirred at room temperature for 10 hours under an atmosphere of hydrogen. The reaction mixture was filtered through celite and evaporated. The residue was partitioned between water and DCM, the organic layers dried over sodium sulphate and evaporated to give yellow which crystallised on standing. ¹H NMR (400 MHz, CDCl₃) δ ppm 1.11 (t, J=7.0 Hz, 3H), 1.53-1.67 (m, 1H), 1.89 (d, J=2.3 Hz, 1H), 2.51-2.73 (m, 2H), 3.40-3.57 (m, 4H), 3.99-4.08 (m, 1H), 6.10 (d, J=2.7 Hz, 1H), 6.22 (dd, J=8.2, 2.34 Hz, 1H), 6.79 (d, J=8.2 Hz, 1H), 9.08 (s, 1H).

### Step AG.3: 7-Benzyloxy-2-ethoxymethyl-chroman

A solution of (7-benzyloxy-chroman-2-yl)-methanol (Step AG.4, 5.38 g, 19.90 mmol) and dimethoxyethane (0.21 ml, 1.99 mmol) in DMF (28 ml) was added dropwise to a suspension of sodium hydride (60% dispersion in mineral oil, 1.35 g, 33.8 mmol) in DMF (14 ml) at room temperature. After stirring for 3.5 hours the reaction mixture was cooled to 0° C. and ethyl iodide (2.73 ml, 33.8 mmol) in DMF (28 ml) added. After stirring for a further 1 hour at room temperature the reaction mixture was partitioned between DCM and water, the organic layers dried over sodium sulphate and evaporated to give the title compound as a yellow oil. ¹H NMR (400 MHz, DMSO) δ ppm 1.11 (t, J=7.0 Hz, 3H), 1.52-1.74 (m, 1H), 1.84-2.01 (m, 1 H), 2.51-2.77 (m, 2H), 3.39-3.63 (m, 4H), 3.97-4.16 (m, 1H), 5.02 (s, 2H), 6.37 (d, J=2.7 Hz, 1H), 6.46 (dd, J=8.4, 2.5 Hz, 1H), 6.91 (d, J=8.2 Hz, 1H), 7.25-7.44 (m, 5H).

### Step AG.4: (7-Benzyloxy-chroman-2-yl)-methanol

Lithium borohydride in THF (21.2 ml of a 2M solution, 45.2 mmol) was added portionwise over 40 minutes to a solution of 7-benzyloxy-chroman-2-carboxylic acid ethyl ester (Step AG.5, 7.00 g, 17.7 mmol) in THF (65 ml) at 0° C. and the mixture stirred for 15.5 hours at room temperature. Further lithium borohydride in THF (21.2 ml of a 2M solution, 45.2 mmol) was added and the reaction mixture stirred at room temperature for 6.5 hours before quenching with ethyl acetate followed by water. Extraction with ethyl acetate, drying of the organic layers over MgSO₄ and evaporation gave the title compound as a pale-yellow oil. ¹H NMR (400 MHz, DMSO) δ ppm 1.52-1.67 (m, 1H), 1.88-1.99 (m, 1H), 2.51-2.75 (m, 2H), 3.45-3.61 (m, 2H), 3.86-3.97 (m, 1H), 4.83 (t, J=5.7 Hz, 1H), 5.01 (s, 2H), 6.34 (d, J=2.3 Hz, 1H), 6.45 (dd, J=8.2, 2.7 Hz, 1H), 6.91 (d, J=8.6 Hz, 1H), 7.32-7.42 (m, 5H).

### Step AG.5: 7-Benzyloxy-chroman-2-carboxylic acid ethyl ester

A mixture of 7-hydroxy-chroman-2-carboxylic acid ethyl ester (Step AG.64.44 g, 20.0 mmol), benzyl bromide (5.75 ml, 50.0 mmol), K₂CO₃ (6.9 g, 50.0 mmol) and DMF (20 ml) was stirred at room temperature for 65 hours. The reaction mixture was partitioned between water and ether, the combined organic layers washed with brine and dried over sodium sulphate. Evaporation gave the title compound as a yellow oil. ¹H NMR (400 MHz, CDCl₃) δ ppm 1.29 (t, J=7.2 Hz, 3H), 2.11-2.20 (m, 1H), 2.25 (dd, J=6.4, 3.7 Hz, 1H), 2.65-2.81 (m, 2H), 4.26 (q, J=7.0 Hz, 2H), 4.69 (dd, J=7.4, 3.5 Hz, 1H), 5.02 (s, 2H), 6.53-6.60 (m, 2H), 6.92 (d, J=8.6 Hz, 1H), 7.29-7.43 (m, 6H).

### Step AG.6: 7-Hydroxy-chroman-2-carboxylic acid ethyl ester

A suspension of Pd/C (10% Engelhard 4505, 4.2 g) in a solution of ethyl 7-hydroxy-4-oxo-4H-chromene-2-carboxylate (25 g, 107 mmol) in acetic acid (260 ml) was stirred for 36.5 hours at room temperature under a 3.5 bar pressure of hydrogen. Filtration through celite, washing with ethyl acetate and evaporation gave a yellow oil to which was added hexane (100 ml) to precipitate the title compound as a white solid which was collected by filtration. ¹H NMR (400 MHz, CDCl₃) d ppm 1.29 (t, J=7.2 Hz, 3H), 2.07-2.31 (m, 2H), 2.58-2.82 (m, 2 H), 4.25 (q, J=7.0 Hz, 2H), 4.69 (dd, J=7.4, 3.5 Hz, 1H), 4.92 (s, 1H), 6.38 (dd, J=8.2, 2.7 Hz, 1H), 6.45 (d, J=2.7 Hz, 1H), 6.87 (d, J=8.2 Hz, 1H).

### Intermediate AH (an alternative route to Intermediate A): 4,4,5,5-tetramethyl-2-(2-phenylchroman-7-yl)-1,3,2-dioxaborolane

A solution of 2-phenylchroman-7-yl trifluoromethanesulfonate (Step AH.1, 1.47 g, 4.10 mmol) in dioxane (15 mL) was treated with bis(pinacolato)diboron (1.04 g, 4.10 mmol), potassium acetate (2.0 g, 12.3 mmol) and Pd(dppf)Cl₂ (375 mg, 0.41 mmol). The solution was purged with N₂ (g) for 3 minutes and then sealed and heated at 90° C. for 15 h. The reaction was cooled to room temperature and poured into saturated ammonia chloride aqueous solution. The mixture was extracted with ethyl acetate (3×30 mL). The organic layers were combined, washed with brine, and dried over sodium sulfate. After concentration, the residue was purified with silica gel column chromatography (10-20% gradient ethyl acetate in hexanes) to afford the title compound as white solid. MS m/z 337 (M+H⁺) (Method M).

### Step AH.1: 2-phenylchroman-7-yl trifluoromethanesulfonate

To a mixture of 2-phenylchroman-7-ol (Step AH.2, 1.46 g, 6.46 mmol) and triethyl amine (2.70 mL, 19.4 mmol) in DCM (20 mL) cooled to 0° C., was added N-phenyl-bis-trifluoromethanesulfonimide (2.53 g, 7.1 mmol) in DCM (5 mL) slowly via syringe. The mixture was stirred at room temperature overnight. After concentration, the residue was purified with silica gel flash column chromatography (0-20% gradient ethyl acetate in hexanes) to afford the title compound as colorless oil. MS m/z 359 (M+H⁺) (Method M).

### Step AH.2: 2-Phenylchroman-7-ol

2-Phenylchroman-4,7-diol obtained from Step AH.3 was dissolved in DCM (20 mL). To the solution was slowly added triethylsilane (5.82 g, 50 mmol). After stirring for 5 min, TFA (5 mL) was added drop by drop to the reaction mixture. The mixture was stirred at room temperature for 30 min. The mixture was concentrated. The residue was dissolved in ethyl acetate, and sequentially washed with sodium carbonate aqueous solution and brine and then dried over Na₂SO₄. After concentration, the residue was purified with silica gel flash column chromatography (0-30% gradient ethyl acetate in hexanes) to afford the title compound as colorless oil. MS m/z 227 (M+H⁺) (Method M).

### Step AH.3: 2-Phenylchroman-4,7-diol

To a solution of 7-hydroxyflavanone (2 g, 8.33 mmol) in THF (30 mL) cooled in an ice bath, was added Borane-THF in THF 12.5 mL) via syringe. The mixture was gradually warmed to room temperature, and continued to stir for 2 h. The reaction was cooled to 0° C., and quenched by adding water slowly until gas evolution ceased. The mixture was concentrated and the residue was partitioned between ethyl acetate and saturated ammonium chloride aqueous solution. The aqueous layer was washed with ethyl acetate.

The organic layers were combined, washed with brine and then dried over Na₂SO₄. After concentration, the title compound was obtained as white solid (MS m/z 225 (M−H₂O+H⁺) (Method M)) and was used directly for next step (Step AH.2) without further purification.

Intermediates AI and AJ: (±)-4,4,5,5-tetramethyl-2-(2-(trans-tetrahydrofuran-2-yl)chroman-7-yl)-1,3,2-dioxaborolane and (±)-4,4,5,5-tetramethyl-2-(2-(cis-tetrahydrofuran-2-yl)chroman-7-yl)-1,3,2-dioxaborolane

The title compounds were prepared in a similar manner to Intermediate AH starting from (±)-trans-2-(tetrahydrofuran-2-yl)chroman-7-yl trifluoromethanesulfonate and (±)-cis-2-(tetrahydrofuran-2-yl)chroman-7-yl trifluoromethanesulfonate (obtained in Steps A1.1 and AJ.1) respectively. MS m/z 331.2 (M+H⁺) (Method M).

Steps A1.1 and AJ.1: (±)-2-(trans-tetrahydrofuran-2-yl)chroman-7-yl trifluoromethanesulfonate and (±)-2-(cis-tetrahydrofuran-2-yl)chroman-7-yl trifluoromethanesulfonate

The title compounds were prepared in a similar manner to Intermediate AH, Step AH.1 starting from (±)-2-(trans-tetrahydrofuran-2-yl)chroman-7-ol and (±)-2-(cis-tetrahydrofuran-2-yl)chroman-7-ol (obtained in Steps A1.2 and AJ.2) respectively. MS m/z 353.1 (M+H⁺) (Method M).

Steps AI.2 and AJ.2: (±)-2-(trans-tetrahydrofuran-2-yl)chroman-7-ol and (±)-2-(cis-tetrahydrofuran-2-yl)chroman-7-ol

To a solution of 2-(furan-2-yl)chroman-4,7-diol (Step A1.3, 300 mg, 1.29 mmol) in acetic acid was added Pd/C (30 mg). The reaction mixture was degassed and purged with H₂ for several minutes and then stirred under 1 atm. Hydrogen gas overnight. The mixture was filtered and concentrated. The residue was purified with silica gel column chromatography (20-30% gradient of ethyl acetate in hexanes) to afford the trans isomer as the first eluent, and the cis isomer as the second eluent. Both had the same ESMS m/z 221.1 (M+H⁺) (Method M).

### Step A1.3: 2-(furan-2-yl)chroman-4,7-diol

The title compound was prepared via a similar sequence as described for Intermediate U, Steps U.2, U.3 and U.4, starting from appropriate starting materials. MS m/z 233.0 (M+H⁺) (Method M).

### Intermediate AK: 7-(cis-4-aminocyclohexyl)-5-(2-phenylchroman-7-yl)-7H-pyrrolo[2,3-d]pyrimidin-4-amine

To a solution of cis-tert-butyl-4-(4-amino-5-(2-phenylchroman-7-yl)-7H-pyrrolo[2,3-d]pyrimidin-7-yl)cyclohexylcarbamate (Step AK.1, 150 mg, 0.28 mmol) in DCM (3 mL), was slowly added TFA (1 mL). The mixture was stirred at room temperature for 1 hour. After evaporation of the solvent using a steady stream of air, the TFA salt of the title compound was obtained as a yellow oil, MS m/z 440.2 (M+H⁺) (Method M).

### Step AK.1: cis-tert-butyl-4-(4-amino-5-(2-phenylchroman-7-yl)-7H-pyrrolo[2,3-d]pyrimidin-7-yl)cyclohexylcarbamate

A mixture of cis-tert-butyl-4-(4-amino-5-iodo-7H-pyrrolo[2,3-d]pyrimidin-7-yl)cyclohexylcarbamate (Step AK.2, 100 mg, 0.22 mmol), (R,S)-2-Phenyl-7-(4,4,5,5-tetramethyl-[1,3,2]dioxaborolan-2-yl)chroman (Intermediate AH, 81 mg, 0.24 mmol), tetrakis(triphenylphosphine)palladium (13 mg, 5% mol), sodium carbonate (93 mg, 0.88 mmol), DMF (4 ml) and water (1 ml) was heated in a sealed vessel at 90° C. for 5 hours under an argon atmosphere. After cooling, water was added and the mixture extracted 3× with DCM, dried over sodium sulphate and evaporated to give the crude product. Purification by silica gel chromatography, eluting with a 0-10% gradient of methanol in DCM gave the title compound. ESMS m/z 540.2 (M+H⁺) (Method M).

### Step AK.2: cis-tert-butyl-4-(4-amino-5-iodo-7H-pyrrolo[2,3-d]pyrimidin-7-yl)cyclohexylcarbamate

A suspension of cis-tert-butyl-4-(4-chloro-5-iodo-7H-pyrrolo[2,3-d]pyrimidin-7-yl)cyclohexylcarbamate (Step AK.3, 500 mg, 0.48 mmol) in ammonium hydroxide (2 mL) and ethanol (1 mL) was heated in a microwave at 130° C. for 1 h. The reaction was cooled. The solid was collected by filtration to afford the title compound as a light yellow solid. ESMS m/z 458 (M+H⁺) (Method M).

### Step AK.3: cis-tert-butyl-4-(4-chloro-5-iodo-7H-pyrrolo[2,3-d]pyrimidin-7-yl)cyclohexylcarbamate

To a mixture to 4-chloro-5-iodo-7H-pyrrolo[2,3-d]pyrimidine (2.79 g, 10 mmol), trans-tert-butyl-4-hydroxycyclohexylcarbamate (2.15 g, 10 mmol), and triphenyphosine (5.2 g, 20 mmol) in anhydrous THF (10 mL) at 0° C., was added DIAD (2.95 mL, 15 mmol). The mixture was stirred at room temperature for 15 hr. The solid formed was filtered, the filtrate was concentrated and purified with silica gel flash column chromatography (25-30% gradient of ethyl acetate in hexanes) to afford the title compound as a white solid, ESMS m/z 477 (M+H⁺) (Method M).

### Intermediate AL: 4-(4-amino-5-(2-phenylchroman-7-yl)-7H-pyrrolo[2,3-d]pyrimidin-7-yl)cyclohexanone

The title compound was prepared in a similar manner to Intermediate AK, Step AK.1 starting from 4-(4-amino-5-iodo-7H-pyrrolo[2,3-d]pyrimidin-7-yl)cyclohexanone (Step AL.1) and Intermediate AH. MS m/z 439.2 (M+H⁺) (Method M).

### Step AL.1: 4-(4-amino-5-iodo-7H-pyrrolo[2,3-d]pyrimidin-7-yl)cyclohexanone

To a mixture of 5-iodo-7-(1,4-dioxaspiro[4.5]decan-8-yl)-7H-pyrrolo[2,3-d]pyrimidin-4-amine (Step AL.2, 430 mg, 1.08 mmol) in acetone/water (8/8 mL), was added oxalic acid dihydrate (274 mg, 2.1 mmol). The mixture was heated at 60° C. overnight. After cooling to room tem-perature, the mixture was concentrated to ˜half of the volume. The precipitate formed was collected by filtration, washed with water and dried under vacuum to afford the title compounds as a biege solid. MS m/z 357.0 (M+H⁺) (Method M).

### Step AL.2: 5-iodo-7-(1,4-dioxaspiro[4.5]decan-8-yl)-7H-pyrrolo[2,3-d]pyrimidin-4-amine

The title compound was prepared in a similar manner to Intermediate AK, Step AK.2 starting from 4-chloro-5-iodo-7-(1,4-dioxaspiro[4.5]decan-8-yl)-7H-pyrrolo[2,3-d]pyrimidine (Step AL.3). MS m/z 401.0 (M+H⁺) (Method M)

### Step AL.3: 4-chloro-5-iodo-7-(1,4-dioxaspiro[4.5]decan-8-yl)-7H-pyrrolo[2,3-d]pyrimidine.

The title compound was prepared in a similar manner to Intermediate AK, Step AK.3 starting from 4-chloro-5-iodo-7H-pyrrolo[2,3-d]-pyrimidine and 1,4-dioxaspiro[4.5]decan-8-ol. MS m/z 419.9 (M+H⁺) (Method M).

### Intermediate AM: 7-[cis-4-(1,1-dioxo-1λ6-thiomorpholin-4-yl)cyclohexyl]5-iodo-7H-pyrrolo[2,3-d]pyrimidin-4-ylamine

To a mixture of 7-(cis-4-aminocyclohexyl)-5-iodo-7H-pyrrolo[2,3-d]pyrimidin-4-amine (Step AM.1, 80 mg, 0.22 mmol), triethyl amine (153 uL, 1.1 mmol) in ethanol (1 mL) was added divinyl sulfone (40 mg, 0.33 mmol). The mixture was stirred at room temperature for 2 hours. The mixture was concentrated and purified with silica gel chromatography (5% MeOH in DCM with 0.1 N NH₃) to afford the title compound as a white solid, MS m/z 476.0 (M+H⁺) (Method M).

### Step AM.1: 7-(cis-4-aminocyclohexyl)-5-iodo-7H-pyrrolo[2,3-d]pyrimidin-4-amine

The title compound was obtained as its TFA salt from removal of the Boc group from Intermediate AK, Step AK.2 using DCM/TFA (5:1). MS m/z 358.0 (M+H⁺) (Method M).

### Intermediate AN: N-(cis-4-(4-amino-5-iodo-7H-pyrrolo[2,3-d]pyrimidin-7-yl)cyclohexyl)acetamide

To a mixture of acetic acid (45 mg, 0.75 mmol), DIEA (0.35 mL), and HATU (280 mg, 0.75 mmol) in DCM (3 mL), was added 7-(cis-4-aminocyclohexyl)-5-iodo-7H-pyrrolo[2,3-d]pyrimidin-4-amine (Intermediate AM, Step AM.1,180 mg, 0.5 mmol) in DCM (0.5 mL). The mixture was stirred for 2 hours, and concentrated. The residue was purified with silica gel chromatography (5% methanol in DCM with 0.1 N NH₃) to afford the title compound. MS m/z 400.0 (M+H⁺) (Method M).

### Intermediate AO: 7-[cis-3-((S,S)-2,2-dioxo-2λ6-thia-5-aza-bicyclo[2,2,1]hept-5-ylmethyl)-cyclobutyl]-5-iodo-7H-pyrrolo[2,3-d]pyrimidin-4-ylamine

The title compound was prepared in a manner similar to 7-(cis-3-((1S,4S)-2-Thia-5-azabicyclo[2.2.1]heptan-5-ylmethyl)cyclobutyl)-5-iodo-7H-pyrrolo[2,3-d]pyrimidin-4-amine (Intermediate AS) starting from cis-3-(4-Amino-5-iodo-pyrrolo[2,3-d]pyrimidin-7-yl)-cyclobutyl]-methanol (Intermediate J), and utilizing 2-thia-5-aza-bicyclo[2.2.1]heptane 2,2-dioxide (Prepared from trans-L-Prolinol via the method described in Huang, X. et al. Bioorg. Med. Chem. Lett. 2009, 19, 4130-4133). MS m/z 474.0 (M+H⁺) (Method M).

### Intermediate AP: 4,4,5,5-tetramethyl-2-(spiro[chroman-2,1′-cyclohexane]-7-yl)-1,3,2-dioxaborolane

The mixture of spiro[chroman-2,1′-cyclohexane]-7-yl trifluoromethanesulfonate (Step AP.1, 593 mg, 1.69 mmol), 4,4,4′,4′,5,5,5′,5′-octamethyl-2,2′-bi(1,3,2-dioxaborolane) (473 mg, 1.86 mmol), Pd(dppf)Cl2 (124 mg, 0.17 mmol), and KOAc (332 mg, 3.3 mmol) in 5.0 mL of 1,4-dioxane was degassed and heated at 90° C. over night. The reaction mixture was partitioned between EtOAc and brine, the collected organic extracts were dried (Na₂SO₄), concentrated in vacuo, and purified by silica chromatography (EtOAc/Hexanes:1/9) to afford the title compound. MS m/z 329.2 (M+H⁺) (Method M).

### Step AP.1: spiro[chroman-2,1′-cyclohexane]-7-yl trifluoromethanesulfonate

Under nitrogen, to the 0° C. solution of spiro[chroman-2,1′-cyclohexan]-7-ol (Step AP.2, 429 mg, 1.97 mmol) in DCM (10.0 mL) was sequentially added Et₃N (0.4 mL, 2.95 mmol), and PhNTf₂ (774 mg, 2.17 mmol). The reaction was allowed to gradually warm to room temperature and stirred overnight. The reaction mixture was partitioned between DCM and brine, the collected organic extracts were dried (Na₂SO₄), concentrated in vacuo, and purified by silica chromatography (EtOAc/Hexanes:1/4) to afford the title compound. MS m/z 351.1 (M+H⁺) (Method M).

### Step AP.2: spiro[chroman-2,1′-cyclohexan]-7-ol

To the solution of spiro[chroman-2,1-cyclohexane]-4,7-diol (Step AP.3, 1.02 g, 4.36 mmol) in DCM (15 mL) at room temperature was added Et₃SiH (6.9 mL, 43.0 mmol), and stirred 5 min. TFA (3.0 mL) was then added to the reaction which was then stirred at room temperature for an additional 30 min. The reaction mixture was concentrated in vacuo and then partitioned between EtOAc and Saturated aqueous NaHCO₃. The collected organic extracts were washed with brine, dried (Na₂SO₄), concentrated in vacuo, and purified by silica chromatography (EtOAc/Hexanes:1/4) to afford the title compound. MS m/z 219.2 (M+H⁺) (Method M).

### Step AP.3: spiro[chroman-2,1′-cyclohexane]-4,7-diol

To the 0° C. solution of 7-hydroxyspiro[chroman-2,1′-cyclohexan]-4-one (Purchased from Princeton BioMolecular Research Inc., 1.0 g, 4.3 mmol) in THF (15 mL) was slowly added BH₃-THF (1.0 M in THF, 6.46 mL, 6.46 mmol). The reaction mixture was allowed to gradually warm to room temperature and stirred for 30 min. The reaction was cooled to 0° C., then saturated aqueous NH₄Cl was slowly added until gas evolution ceased. The mixture was partitioned between EtOAc and brine, the collected organic extracts were dried (Na₂SO₄), concentrated in vacuo, and the resulting crude title product was used directly in the next step (Step AP.2) without further purification or characterization.

### Intermediate AQ: 2-(7-(4,4,5,5-tetramethyl-1,3,2-dioxaborolan-2-yl)chroman-2-yl)pyridine

The mixture of 2-(7-bromochroman-2-yl)pyridine (Step AQ.1, 73 mg, 0.25 mmol), 4,4,4′,4′,5,5,5′,5′-octamethyl-2,2′-bi(1,3,2-dioxaborolane) (63 mg, 0.25 mmol), Pd₂(dba)₃ (11.4 mg, 0.01 mmol), tricyclohexylphosphine (7.0 mg, 0.03 mmol) and KOAc (49 mg, 0.5 mmol) in 2.0 mL of 1,4-dioxane was degassed and then heated at 80° C. for 10 h. The reaction mixture was partitioned between EtOAc and brine, the collected organic extracts were dried (Na₂SO₄), concentrated in vacuo and purifed with silica chromatography (EtOAc/Hexanes:1/4) to afford the title compound. MS m/z 338.2 (M+H⁺) (Method M).

### Step AQ.1: 2-(7-Bromochroman-2-yl)pyridine

To a solution of 7-bromo-2-(pyridin-2-yl)chroman-4-one (Step AQ.2, 400 mg, 1.32 mmol) in TEA (4 mL) was added triethylsilane (1.0 mL, 6.2 mmol). After stirring at room temperature for 14 h, additional triethylsilane (0.5 mL, 3.1 mmol) was added. The reaction was then stirred at 65° C. Additional triethylsilane was added until LCMS indicated the completion of the reaction. The solvent was evaporated. The residue was basified with saturated aqueous NaHCO₃ and extracted with dichloromethane. The dichloromethane extracts were washed with brine, dried over Na₂SO₄ and evaporated. The resulting residue was purified by flash chromatography (SiO₂, 0-5% gradient of MeOH in DCM) to give the title compound as an off-white solid. ¹H NMR (400 MHz, CDCl₃) δ 8.61 (d, J=4.8 Hz, 1H), 7.75 (dt, J=2.0, 7.6 Hz, 1H), 7.53 (d, J=8.0 Hz, 1H), 7.25 (ddd, J=1.2, 4.8, 7.6 Hz, 1H), 7.15 (d, J=2.0 Hz, 1H), 7.03 (dd, J=1.6, 8.0 Hz, 1H), 6.96 (d, J=8.4 Hz, 1H), 5.23 (dd, J=2.8, 9.2 Hz, 1H), 2.91 (m, 1H), 2.72 (t, J=4.8 Hz, 1H), 2.68 (t, J=5.2 Hz, 1H), 2.46 (m, 1H), 2.14 (m, 1H); MS m/z 290.0/292.0 (M+H⁺) (Method M).

### Step AQ.2: 7-Bromo-2-(pyridin-2-yl)chroman-4-one

A mixture of (E)-1-(4-bromo-2-hydroxyphenyl)-3-(pyridin-2-yl)prop-2-en-1-one (Step AQ.3, 1.02 g, 3.37 mmol) and sodium acetate (2.66 g, 32.4 mmol) in ethanol (15 mL) was stirred at room temperature overnight. The reaction was diluted with water (30 mL) and extracted with dichloromethane (3×50 mL). The combined dichloromethane extracts were washed with brine (20 mL), dried over Na₂SO₄ and evaporated. The resulting residue was purified by flash chromatography (SiO₂, 20-50% gradient of EtOAc in hexanes) to give the title compound as a light yellow solid. ¹HNMR (400 MHz, CDCl₃) δ 8.62 (m, 1H), 7.89 (m, 2H), 7.57 (d, J=8.0 Hz, 1H), 7.30 (m, 2H), 7.20 (dd, J=2.0, 8.4 Hz, 1H), 5.62 (dd, J=4.0, 11.2 Hz, 1H), 3.24-3.10 (m, 2H); MS m/z 304.0/306.0 (M+H⁺) (Method M).

### Step AQ.3: (E)-1-(4-Bromo-2-hydroxyphenyl)-3-(pyridin-2-yl)prop-2-en-1-one

To a solution of 1-(4-bromo-2-hydroxyphenyl)ethanone (3.00 g, 14.0 mmol) and picolinaldehyde (1.50 g, 15.6 mmol) in methanol (50 mL) was added potassium hydroxide (2.70 g, 48.2 mmol). After stirring at room temperature for 40 h, the reaction was then stirred at 40° C. for additional 10 h. The solvent was removed by evaporation and HCl solution (1N, 50 mL) was added. The yellow precipitate was collected by filtration, washed with water, dried and the resulting crude title product was used directly in the next step (Step AQ.2) without further purification. MS m/z 304.0/306.0 (M+H⁺) (Method M).

### Intermediate AR: N-((cis-3-(4-amino-5-iodo-7H-pyrrolo[2,3-d]pyrimidin-7-yl)cyclobutyl)methyl)acetamide

The mixture of 7-(cis-3-aminomethyl-cyclobutyl)-5-iodo-7H-pyrrolo[2,3-d]pyrimidin-4-ylamine (Step AR.1, 274 mg, 0.8 mmol), acetic acid (48 mg, 0.8 mmol), HATU (367 mg, 0.96 mmol) and DIEA (0.17 mL, 0.96 mmol) in 5.0 mL of DMF was stirred at room temperature for 2 h. The reaction mixture was partitioned between EtOAc and brine, the collected organic extracts were dried (Na₂SO₄), concentrated in vacuo, and purified by silica chromatography (EtOAc/Hexanes:1/1) to afford the title compound. MS m/z 386.0 (M+H⁺) (Method M).

### Step AR.1: 7-(3-Aminomethyl-cyclobutyl)-5-iodo-7H-pyrrolo[2,3-d]pyrimidin-4-ylamine

Triphenylphosphine (833 mg, 3.18 mmol) was added to a mixture of 7-(3-azidomethyl-cyclobutyl)-5-iodo-7H-pyrrolo[2,3-d]pyrimidin-4-ylamine (Step AR.2, 920 mg, 2.12 mmol), ammonium hydroxide solution (25%, 1.32 ml, 8.47 mmol), water (1.4 ml), methanol (7 ml) and THF (7 ml). The reaction mixture was stirred overnight at room temperature, then diluted with water, extracted 2× with ethyl acetate, the combined organic phases washed with brine, dried over sodium sulphate and evaporated. Purification of the residue by flash column chromatography, eluting with a gradient of methanol in DCM containing 1% concentrated ammonia solution, gave the title compound as a white solid. ¹H NMR (400 MHz, DMSO) δ ppm 1.74 (s, br, 2H), 2.06-2.18 (m, 3H), 2.32-2.39 (m, 2H), 2.57-2.60 (m, 2H), 4.95-5.02 (m, 1H), 6.59 (s, br, 2H), 7.68 (s, 1H), 8.08 (s, 1H).

### Step AR.2: 7-(cis-3-azidomethyl-cyclobutyl)-5-iodo-7H-pyrrolo[2,3-d]pyrimidin-4-ylamine

A mixture of toluene-4-sulfonic acid cis-3-(4-amino-5-iodo-pyrrolo[2,3-d]pyrimidin-7-yl)-cyclobutylmethyl ester (Step AR.3, 18.0 g, 18.1 mmol), sodium azide (4.70 g, 72.2 mmol) and DMF (60 ml) was heated at 65° C. for 1 hour. The cooled reaction mixture was diluted with water, extracted 3× with ethyl acetate, the combined organic phases washed with brine, dried over magnesium sulphate and evaporated to give the title compound as a yellow solid. HPLC/MS t_{R} 0.97 min, M+H 369.9 (Method X).

### Step AR.3: Toluene-4-sulfonic acid cis-3-(4-amino-5-iodo-pyrrolo[2,3-d]pyrimidin-7-yl)-cyclobutylmethyl ester para-Toluene sulphonyl chloride (11.52 g, 60.4 mmol) was added portion-wise over 45 minutes to a solution of [cis-3-(4-amino-5-iodo-pyrrolo[2,3-d]pyrimidin-7-yl)-cyclobutyl]-methanol (Intermediate J, 7.0 g, 20.14 mmol) in pyridine (20 ml) cooled at −20° C. After 18 hours at −25° C. the reaction mixture was partitioned between 1N sulphuric acid and DCM cooled air 0° C., extracted 2× with DCM, the combined organic layers dried over sodium sulphate and evaporated to give the title compound as a yellow solid. HPLC/MS t_{R} 1.12 min, M+H 498.9 (Method X).

### Intermediate AS: 7-(cis-3-((1S,4S)-2-Thia-5-azabicyclo[2.2.1]heptan-5-ylmethyl)cyclobutyl)-5-iodo-7H-pyrrolo[2,3-d]pyrimidin-4-amine

A suspension of cis-3-(4-Amino-5-iodo-pyrrolo[2,3-d]pyrimidin-7-yl)-cyclobutyl]-methanol (Intermediate J, 200 mg, 0.58 mmol) and 2-Iodoxybenzoic acid (325 mg, 1.16 mmol) in anhydrous MeCN (10 mL) was stirred at 80° C. After 1 h, LCMS (Method M) showed complete conversion of the primary alcohol in the starting material to the corresponding aldehyde. MS m/z 343.1 (M+H+) (Method M). To the above reaction mixture were added NaBH(OAc)₃ (369 mg, 1.74 mmol), (1S,4S)-2-thia-5-azabicyclo[2.2.1]heptane HCl salt (Prepared from trans-L-Prolinol via the method described in Huang, X. et al. Bioorg. Med. Chem. Lett. 2009, 19, 4130-4133, 124 mg, 0.82 mmol), DIEA (987 uL, 2.59 mmol) and dichloromethane (10 mL). After stirring at room temperature for 30 min, the solvent was evaporated. Water (20 mL) was added. The mixture was extracted with dichloromethane (3×30 mL). The combined dichloromethane extracts were sequentially washed with water (10 mL), brine (10 mL), dried over Na₂SO₄ and evaporated. The resulting residue was purified by flash chromatography (SiO2, 0-10% gradient of MeOH in dichloromethane) to give the title compound as an off-white sol-id. MS m/z 442.0 (M+H⁺) (Method M).

### Intermediate AT: 3-[4-Amino-5-[2-phenyl-chroman-7-yl)-pyrrolo[2,3-d]pyrimidin-7-yl]cyclobutanone

Sodium periodate (91 mg, 0.426 mmol) was added to a stirred mixture of (Z)-3-[4-amino-5-(2-phenyl-chroman-7-yl)-pyrrolo[2,3-d]pyrimidin-7-yl]-1-hydroxymethyl-cyclobutanol (Example 41, 145 mg, 0.328 mmol) in 13.3 mL of THF/H₂O (3/1, v:v). the reaction mixture was stirred for 19 h at rt, diluted with ethyl acetate/brine and extracted with ethyl acetate. The combined organic extracts were washed with brine, dried (Na₂SO₄), flitered and concen-trated. The residue was purified by flash chromatography (DCM/MeOH/NH4OH c., 200:10:1) to afford of the title compound as a beige foam. HPLC/MS (Method Z) t_{R} 1.01 minutes, M+H 411.2.

Intermediate AU: (cis)-5-Iodo-7-[3-(1-oxo-thiomorpholin-4-ylmethyl)-cyclobutyl]-7H-pyrrolo[2,3-d]pyrimidin-4-ylamine

To the stirred solution of (cis)-[3-(4-amino-5-iodo-pyrrolo[2,3-d]pyrimidin-7-yl)cyclobutyl]methanol (Intermediate J: 348 mg, 1.0 mmol) and acetonitrile (70 mL) was added IBX (Atlantic SciTech 86900: 561 mg, 2.0 mmol, 2 eq). The reaction mixture was stirred for 1 hour at 80° C. The reaction mixture was filtered at 40° C. and the filtrate was concentrated. To the residue was added subsequently DCM (50 mL), ethyl-diisopropyl-amine (3.43 mL, 20 mmol, 20 eq), 1-oxide thiomorpholin hydrochloride (312 mg, 2.0 mmol, 2 eq) and sodium triacetoxyborohydride (637 mg, 3.0 mmol, 3 eq) with stirring at room temperature. The reaction mixture was stirred for 1 hour at ro temperature and then partitioned between NaHCO₃ 1M and EtOAc. The combined organic layers were washed with water and brine, dried (Na₂SO₄), filtered and concentrated. The residue was purified by silica gel column chromatography (DCM/MeOH/NH₃^{aq}, 200:20:1) to afford the title compound as pale yellow crystals: HPLC/MS (Method Z) t_{R} 0.92 minutes, M+H 446.2. TLC; R_{f}=0.26 (DCM/MeOH/NH₃^{aq}, 200:20:1).

### III Chemical Synthesis—Compounds According to the Invention

### Example 1

### (R,S)-{cis-3-[4-Amino-5-(2-phenyl-chroman-7-yl)-pyrrolo[2,3-d]pyrimidin-7-yl]-cyclobutyl}-methanol

A stirred mixture of [cis-3-(4-amino-5-iodo-pyrrolo[2,3-d]pyrimidin-7-yl)-cyclobutyl]-methanol (Intermediate J, 0.50 g, 1.45 mmol), (R,S)-2-phenyl-7-(4,4,5,5-tetramethyl-[1,3,2]dioxaborolan-2-yl)chroman (Intermediate A, 0.49 g, 1.45 mmol), tetrakis(triphenylphosphine)palladium(0) (0.17 g, 0.15 mmol) and sodium carbonate (0.31 g, 2.91 mmol) in DMF (4 ml) and water (2 ml) was heated under an argon atmosphere for 17 hours at 80° C. The cooled reaction mixture was partitioned between water and dichloromethane, the organic layers were washed with brine, dried over sodium sulphate and evaporated to give the crude product which was purified by reversed phase preparative chromatography (Method R) to give the title compound as a green/white foam. MS (Method L) M+H 427.1 (100%).

### Example 2

### (R,S)-{Trans-3-[4-amino-5-(2-phenyl-chroman-7-yl)-pyrrolo[2,3-d]pyrimidin-7-yl]-cyclobutyl}-methanol

A stirred mixture of [trans-3-(4-amino-5-iodo-pyrrolo[2,3-d]pyrimidin-7-yl)-cyclobutyl]-methanol (Intermediate L, 0.20 g, 0.57 mmol), (R,S)-2-phenyl-7-(4,4,5,5-tetramethyl-[1,3,2]dioxaborolan-2-yl)chroman (Intermediate A, 0.20 g, 0.57 mmol), tetrakis(triphenylphosphine)palladium(0) (0.07 g, 0.06 mmol) and sodium carbonate (0.12 g, 1.13 mmol) in DMF (3 ml) and water (1.5 ml) was heated under an argon atmosphere for 21 hours at 80° C. The cooled reaction mixture was partitioned between water and dichloromethane, the organic layers were washed with brine, dried over sodium sulphate and evaporated to give the crude product which was purified by silica gel chromatography, eluting with a gradient of methanol in dichloromethane. The product containing fractions were combined, evaporated and further purified by reversed phase chromatography (Method R). Product containing fractions were eluted through a 300 mg Bond Elut-SCX cartridge and then released by eluting with 7M ammonia in methanol and evaporated to give the title compound as a green/white foam. HPLC/MS (Method Z) t_{R} 1.06 minutes, M+H 427.0 (100%).

### Example 3

### (R,S)-{cis-3-[8-Amino-1-(2-phenyl-chroman-7-yl)-imidazo[1,5-a]pyrazin-3-yl]-cyclobutyl}-methanol

[cis-3-(8-Amino-1-iodo-imidazol[1,5-a]pyrazin-3-yl-cyclobutyl]-methanol (prepared according to US20070129547, 0.18 g, 0.51 mmol) was dissolved in dioxane (3 mL). Water (3 mL), (R,S)-2-phenyl-7-(4,4,5,5-tetramethyl-[1,3,2]dioxaborolan-2-yl)chroman (Intermediate A, 0.22 g, 0.66 mmol), K₃PO₄ (0.43 g, 2.03 mmol) and Pd(PPh₃)₄ (0.12 g, 0.10 mmol) were added and the reaction mixture was flushed with argon and heated to 60° C. for 2 hours. The reaction mixture was allowed to cool and diluted with EtOAc. The organic layer was washed with brine, dried and concentrated. The crude product was purified by flash column chromatography, eluting with a DCM/MeOH gradient to give the title compound as an off-white solid. HPLC/MS (Method Z) t_{R} 0.94 minutes, M+H 427.1 (100%).

### Example 4

### 1-{cis-3,8-Amino-1-((R)-2-phenyl-chroman-7-yl)-imidazo[1,5-a]pyrazin-3-yl]-cyclobutylmethyl}-(S)-pyrrolidine-2-carboxylic acid amide and 1-{cis-3-[8-amino-1-((S)-2-phenyl-chroman-7-yl)-imidazo[1,5-a]pyrazin-3-yl]-cyclobutylmethyl}-(S)-pyrrolidine-2-carboxylic acid amide

(R,S)-cis-3-[8-Amino-1-(2-phenyl-chroman-7-yl)-imidazo[1,5-a]pyrazin-3-yl]-cyclobutanecarbaldehyde (Intermediate N, 40 mg, 0.09 mmol) was dissolved in 1,2-dichloroethane (3 mL) and L-proline amide (5 mg, 0.47 mmol) and acetic acid (54 μL, 0.95 mmol) were added. The reaction mixture was stirred at room temperature for 30 minutes followed by the addition of sodium triactetoxy borohydrate (40 mg, 0.19 mmol). Stirring was continued for a further 48 hours at room temperature. The reaction was diluted with DCM and quenched by addition of saturated aqueous NaHCO₃ solution. The organic layer was se-parated, washed with brine dried and concentrated. The crude material was purified by preparative TLC (SiO₂/MeOH 10%, 90% CH₂Cl₂). HPLC/MS (Method Z) t_{R} 0.72 minute, M+H 523.2 (100%). HPLC_{A}: t_{R} 3.51 min.

### Example 5

### (R,S)—(Z)-3-[8-Amino-1-(2-phenyl-chroman-7-yl)-imidazo[1,5-a]pyrazin-3-yl]-1-hydroxymethyl-cyclobutanol and (R,S)-(E)-3-[8-amino-1-(2-phenyl-chroman-7-yl)-imidazo[1,5-a]pyrazin-3-yl]-1-hydroxymethyl-cyclobutanol

The title compound was prepared in analogy to the procedure described for Example 1 from (Z)-3-(8-Amino-1-iodo-imidazo[1,5-a]pyrazin-3-yl)-1-hydroxymethyl-cyclobutanol and (E)-3-(8-amino-1-iodo-imidazo[1,5-a]pyrazin-3-yl)-1-hydroxymethyl-cyclobutanol (Intermediate N) and (R,S)-2-phenyl-7-(4,4,5,5-tetramethyl-[1,3,2]dioxaborolan-2-yl)chroman (Intermediate A). M+H 444.4 (Method X). ¹H NMR (400 MHz, CD₃OD) δ ppm 7.41-7.26 (m, 7H), 7.08-7.06 (m, 2H), 6.95 (d, 1H), 5.10 (d, 1H), 4.01-3.94 (m, 1H), 3.49 (5, 2H), 3.05-3.00 (m, 1H), 2.82-2.69 (m, 1H), 2.51-2.45 (m, 2H), 2.22-2.19 (m, 2H), 2.07-2.04 (m, 2H).

### Example 6

### (R,S)-7-[cis-3-(1,1-Dioxo-thiomorpholin-4-ylmethyl)-cyclobutyl]-5-(2-phenyl-chroman-7-yl)-7H-pyrrolo[2,3-d]pyrimidin-4-ylamine

A stirred mixture of 5-bromo-7-[cis-3-(1,1-dioxo-thiomorpholin-4-ylmethyl)-cyclobutyl]-7H-pyrrolo[2,3-d]pyrimidin-4-ylamine (Intermediate O, 0.10 g, 0.24 mmol), (R,S)-2-phenyl-7-(4,4,5,5-tetramethyl-[1,3,2]dioxaborolan-2-yl)chroman (Intermediate A, 0.122 g, 0.36 mmol), tetrakis(triphenylphosphine)palladium(0) (0.022 g, 0.019 mmol) and sodium carbonate (0.051 g, 0.483 mmol) in DMF (2 ml) and water (0.1 ml) was heated under an argon atmosphere for 1 hour at 100° C. The cooled reaction mixture was partitioned between water and dichloro-methane, the organic layers were washed with brine, dried over sodium sulphate and evaporated to give the crude product which was purified by silica gel chromatography, eluting with a gradient of methanol in dichloromethane. The product containing fractions were combined, evaporated and further purified by Sepacore flash chromatography (8 g cartridge). Product containing fractions were collected and evaporated to give the title compound as a white foam. HPLC/MS (Method N) t_{R} 1.13 minutes, M+H 544.5 (100%).

### Example 7

### (R,S)-3-[cis-3-(2-Methyl-imidazol-1-ylmethyl)-cyclobutyl]-1-(2-phenyl-chroman-7-yl)-imidazo[1,5-a]pyrazin-8-ylamine

2-Methyl imidazole (23 mg, 0.28 mmol) was dissolved in DMF (2.5 mL) and cooled to 0° C. Sodium hydride (60% oil dispersion, 11 mg, 0.28 mmol) was added and the reaction stirred for 30 minutes at room temperature. (R,S)-toluene-4-sulphonic acid cis-3-[8-amino-1-(2-phenyl-chroman-7-yl)-imidazo[1,5-a]pyrazin-3-yl]-cyclobutylmethyl ester (Intermediate P, 107 mg, 0.18 mmol) was then added and the reaction mixture stirred at 80° C. for 8 hours. The reaction mixture was allowed to cool to room temperature and submitted to aqueous workup. The crude material was purified by flash chromatography (SiO₂; DCM/MeOH; gradient 0-5% MeOH). MS M+H 491.1 (Method X). ¹H NMR (400 MHz, CD₃OD) δ ppm 7.45 (d, 1H), 7.43 (d, 1H), 7.39-7.35 (m, 5H), 7.30 (d, 1H), 7.25 (d, 1H), 7.12 d, 1H), 7.03 (s, 1H), 6.97 (d, 1), 6.82 (d, 1H), 5.16 (d, 1H), 4.04 (d, 2H), 3.83 (dd, 1H), 3.07-3.04 (m, 1H), 2.85-2.81 (m, 2H), 2.60-2.56 (m, 2H), 2.38 (s, 3H), 2.36-2.26 (m, 3H), 2.25-2.08 (m, 1H).

### Examples 8 and 9

### (R,S)-(E)-3-[4-Amino-5-(2-phenyl-chroman-7-yl)-pyrrolo[2,3-d]pyrimidin-7-yl]-1-hydroxymethyl-cyclobutanol and (R,S)—(Z)-3-[4-amino-5-(2-phenyl-chroman-7-yl)-pyrrolo[2,3-d]pyrimidin-7-yl]-1-hydroxymethyl-cyclobutanol

A mixture (2:1) of ((E)-3-(4-amino-5-iodo-pyrrolo[2,3-d]pyrimidin-7-yl)-1-hydroxymethyl-cyclobutanol and (Z)-3-(4-amino-5-iodo-pyrrolo[2,3-d]pyrimidin-7-yl)-1-hydroxymethyl-cyclobutanol) (Intermediates Q and R, 0.3 g, 0.833 mmol) and the racemic mixture of (R,S)-2-phenyl-7-(4,4,5,5-tetramethyl-[1,3,2]dioxaborolan-2-yl)chroman) (Intermediate A, 354 mg, 1 mol), tetrakis(triphenylphosphine)palladium(0) (48.1 mg, 0.042 mmol), Na₂CO₃ (117 mg, 1.67 mmol), and K₃PO₄ (290 mg, 1.67 mmol) were dissolved in degassed DMF (5.55 mL)/H₂O (0.28 mL) under argon. After stirring at 100° C. for 1 hour, the solvent was evaporated under reduced pressure and H₂O added (4 mL). The reaction mixture was extracted with AcOEt (10 mL, 4×) and the combined organic phases were washed with brine (3 mL, 2 x), dried (MgSO4), and concentrated under reduced pressure. The crude product was purified using a Sepacore Control chromatographic separator (Büchi) and 40 g silica gel columns (RediSept) (eluent: CH₂Cl₂→10% MeOH (10% NH₃) in CH₂Cl₂ in 40 minutes). After multiple column runs, the geometric isomers, Examples 8 and 9, were completely separated:

### Example 8

### E-Isomer

Beige solid. MS (method L) M+H=443 (100%). HPLC (Method B): t_{R} 2.51 minutes. TLC (NH₃/MeOH/CH₂Cl₂=1:10:89): R^{F}=0.38. ¹H-NMR (600 MHz, DMSO-d₆): δ ppm 8.13 (s, 1H), 7.47 (s, 1H), 7.55 (d, 2H), 7.41 (t, 2H), 7.34 (t, 1H), 7.22 (d, 1H), 6.94 (d, 1H), 6.74 (s, 1H), 6.15 (s/broad, 2H), 5.34 (quint, 1H), 5.21 (d, 1H), 5.11 (s, 1H), 4.82 (t, 1H), 3.29 (d, 2H), 3.00/2.76 (m/m, 2H), 2.65/2.30 (t/t, 4H), 2.24/2.03 (m/m, 2H). ROESY observed between HO-cyclobutyl and HC-cyclobutyl.

### Example 9

### Z-Isomer

Beige solid. MS (Method L) M+H=443 (100%). HPLC (Method B): t_{R} 2.51 minutes. TLC (NH₃/MeOH/CH₂Cl₂=1:10:89): R^{E}=0.45. ¹H-NMR (600 MHz, DMSO-d₆): δ ppm 8.11 (s, 1H), 7.62 (s, 1H), 7.47 (d, 2H), 7.41 (t, 2H), 7.34 (t, 1H), 7.22 (d, 1H), 6.98 (d, 1H), 6.96 (s, 1H), 6.12 (s/broad, 2H), 5.26 (s, 1H), 5.19 (d, 1H), 4.85 (m, 1H), 3.35 (d, 2H), 3.00/2.79 (m/m, 2H), 2.72/2.41 (t/t, 4H), 2.22/2.02 (m/m, 2H).

### Example 10

### (R,S)-1-{cis-3-[8-Amino-1-(2-phenyl-chroman-7-yl)-imidazo[1,5-a]pyrazin-3-yl]-cyclobutylmethyl}-1H-imidazole-2-carboxylic acid ethyl ester

The title compound was obtained according to the procedure described for Example 7 from (R,S)-toluene-4-sulphonic acid cis-3-[8-amino-1-(2-phenyl-chroman-7-yl)-imidazo[1,5-a]pyrazin-3-yl]-cyclobutylmethyl ester (Intermediate P) and ethylimidazol-2-carboxylate. HPLC/MS (Method N) t_{R} 1.01 minute, M+H 549.24 (100%). ¹H NMR (400 MHz, CD₃OD) δ ppm 7.49-7.29 (m, 9H), 7.14-7.10 (m, 2H), 6.96 (d, 1H), 5.16 (d, 1H), 4.56 (d, 2H), 4.39 (q, 2H), 3.82 (dd, 1H), 3.14-2.90 (m, 2H), 2.95-2.88 (m, 2H), 2.65-2.55 (m, 2H), 2.37-2.29 (m, 3H), 2.12-2.01 (m, 1H), 1.38 (t, 3H).

### Example 11 and 12

### {cis-3-[4-Amino-5-((S)-2-phenyl-chroman-7-yl)-pyrrolo[2,3-d]pyrimidin-7-yl]-cyclobutyl}-methanol and {cis-3-[4-Amino-5-((R)-2-phenyl-chroman-7-yl)-pyrrolo[2,3-d]pyrimidin-7-yl]-cyclobutyl}-methanol

(R,S)-{cis-3-[4-Amino-5-(2-phenyl-chroman-7-yl)-pyrrolo[2,3-d]pyrimidin-7-yl]-cyclobutyl}-methanol (Example 1) was separated by preparative chiral chromatography (IB 250×20 mm, Chiralpak ODH; 90% hexanes/5% MeOH/5% EtOH; flow 10 ml/min) HPLC/MS (Method N) t_{R} 1.04 minutes, M+H 427.0 (100%).

### Example 13

### (R,S)-7-(cis-3-Aminomethyl-cyclobutyl)-5-(2-phenyl-chroman-7-yl)-7H-pyrrolo[2,3-d]pyrimidin-4-ylamine

(R,S)-7-(cis-3-Azidomethyl-cyclobutyl)-5-(2-phenyl-chroman-7-yl)-7H-pyrrolo[2,3-d]pyrimidin-4-ylamine (Intermediate S) (580 mg, 1.0 mmol) was dissolved in THF (4 mL) and water (1 mL) and MeOH (4 mL) were added, followed by PPh₃ (380 mg, 1.5 mmol) and aqueous ammonia solution (25% wt, 0.64 mL) at room temperature. The reaction was allowed to stir for 12 hours at room temperature, then diluted with EtOAc and submitted to aqueous workup. The crude material was purified by flash chromatography (SiO₂, CH₂Cl₂/MeOH; gradient: 0-20% MeOH) to give the title compound as a yellow solid. MS M+H 426.1 (Method X). ¹H NMR (400 MHz, CD₃OD) δ ppm 1.94-2.07 (m, 1H) 2.14-2.23 (m, 2H) 2.23-2.32 (m, 2H), 2.60-2.80 (m, 2H) 2.60-2.80 (m, 2H) 2.90-2.96 (m, 1H) 3.27-3.33 (m, 1H) 5.05 (d, 2H) 6.94 (d, 2H) 7.13 (d, 1H) 7.25-7.29 (m, 1H) 7.29-7.36 (m, 3H) 7.36-7.41 (m, 2H) 8.10 (s, 1H).

### Example 14

### (1-{cis-3-[4-Amino-5-((R)-2-phenyl-chroman-7-yl)-pyrrolo[2,3-d]pyrimidin-7-yl]-cyclobutylmethyl}-(S)-pyrrolidin-2-yl)-methanol and (1-{cis-3-[4-amino-5-[((S)-2-phenyl-chroman-7-yl)-pyrrolo[2,3-d]pyrimidin-7-yl]-cyclobutylmethyl}-(S)-pyrrolidin-2-yl)-methanol Toluene-4-sulphonic acid cis-3-[4-amino-5-(2-phenyl-chroman-7-yl)-pyrrolo[2,3-d]pyrimidin-7-yl]-cyclobutylmethyl ester (Intermediate T, 110 mg, 0.19 mmol) was dissolved in DMF (3 mL) at room temperature. L-Prolinol (78 mg, 0.77 mmol) and DIPEA (135 μL, 0.77 mmol) were added and the reaction mixture stirred at 60° C. for 12 hours. The reaction mixture was allowed to cool to room temperature and then submitted to aqueous workup. The crude product was purified by flash chromatography (SiO₂; DCM/MeOH; gradient 0-50% MeOH). HPLC_{A}: t_{R} 3.71 minutes. HPLCIMS (Method N) t_{R} 0.92 minutes, M+H 510.2 (100%).

### Example 15

### (R,S)-1-{cis-3-[4-Amino-5-(2-phenyl-chroman-7-yl)-pyrrolo[2,3-d]pyrimidin-7-yl]-cyclobutylmethyl}-1H-imidazole-2-carboxylic acid ethyl ester

The title compound was obtained according to the procedure described for Example 10 from toluene-4-sulphonic acid cis-3-[4-amino-5-(2-phenyl-chroman-7-yl)-pyrrolo[2,3-d]pyrimidin-7-yl]-cyclobutylmethyl ester (Intermediate T). HPLCIMS (Method N) t_{R} 0.88 minute, M+H 549.3 (99%). ¹H NMR (400 MHz, CD₃OD) δ ppm 8.11 (s, 1H), 7.44 (d, 2H), 7.36-7.33 (m, 3H), 7.20 £8d, 1H), 7.18 (d, 1H), 7.12 (s, 1H), 6.99-6.96 (m, 2H), 5.13-5.05 (m, 2H), 4.63 (d, 2H), 4.36 (q 2H), 3.10-2.99 (m, 1H), 2.70-2.62 (m, 1H), 2.60-2.56 (m, 1H), 2.55-2.50 (m, 2H), 2.39-2.34 (m, 2H), 2.24-2.20 (m, 1H), 2.07-2.04 (m, 1H), 1.36 (t, 3H).

### Example 16

### (R,S)—N-{cis-3-[4-Amino-5-(2-phenyl-chroman-7-yl)-pyrrolo[2,3-d]pyrimidin-7-yl]-cyclobutylmethyl}-acetamide

(R,S)-7-(cis-3-Aminomethyl-cyclobutyl)-5-(2-phenyl-chroman-7-yl)-7H-pyrrolo[2,3-d]pyrimidin-4-ylamine (Example 13, 105 mg, 0.25 mmol) was dissolved in DCM and cooled to −40° C. under an argon atmosphere. At this temperature acetic anhydride (27 mg, 0.27 mmol) and DIEA (95 μL, 0.54 mmol) were added and stirring continued for a further 48 hours while the reaction mixture was allowed to warm slowly to room temperature. The reaction was then submitted to aqueous workup and the crude material purified by flash chromatography (SiO₂; DCM/MeOH; gradient 0-10% MeOH). HPLC/MS (Method N) t_{R} 1.03 minutes, M+H 468.1 (100%). ¹H NMR (400 MHz, CDCl₃) δ ppm 8.31 (s, 1H), 7.47 (d, 2H), 7.45 (d, 2H), 7.37-7.34 (m, 1H), 7.29 (5, 1H), 7.20 (d, 1H), 7.06 (d, 2H), 6.99 (d, 1H), 6.44 (bs, 1H), 5.65 (bs, 2H), 5.14 (d, 1H), 5.01 (quin, 1H), 3.43 (dd, 2H), 3.08-3.04 (m, 1H), 2.91-2.84 (m, 1H), 2.70-2.66 (m, 2H), 2.57-2.45 (m, 3H). 2.29-2.24 (m, 1H) 2.18-2.13 (m, 2H), 2.09 (s, 3H).

### Example 17

### (R,S)—N-{cis-3-[4-Amino-5-(2-phenyl-chroman-7-yl)-pyrrolo[2,3-d]pyrimidin-7-yl]-cyclobutylmethyl}-methanesulfonamide

(R,S)-7-(cis-3-Aminomethyl-cyclobutyl)-5-(2-phenyl-chroman-7-yl)-7H-pyrrolo[2,3-d]pyrimidin-4-ylamine (Example 13, 105 mg, 0.25 mmol) was dissolved in DCM under an argon atmosphere. Methanesulfonic anhydride (47 mg, 0.27 mmol) and DIEA (95 μL, 0.54 mmol) were added at room temperature and stirring continued for a further 12 hours. The reaction was then submitted to an aqueous workup and the crude material purified by flash chromatography (SiO₂; DCM/MeOH; gradient 0-10% MeOH). HPLC/MS (Method N) t_{R} 1.08 minute, M+H 426.2 (100%). ¹H NMR (400 MHz, CDCl₃) δ ppm 8.40 (s, 1H), 7.47 (d, 2H), 7.43-7.41 (m, 2H), 7.36 (dd, 1H), 7.20 (d, 1H), 7.02 (s, 1H), 6.98-6.96 (m, 2H), 5.65 (bs, 2H), 5.14 (d, 1H), 4.92 (quin, 1H), 3.34 (dd, 2H), 3.07-3.01 (m, 1H), 3.00 (s, 3H), 2.89-2.83 (m, 1H), 2.81-2.77 (m, 2H), 2.71-2.68 (m, 2H), 2.30-2.27 (m, 1H), 2.19-2.12 (m, 1H).

### Example 18

### (R,S)-(1-{cis-3-[4-Amino-5-(2-phenyl-chroman-7-yl)-pyrrolo[2,3-d]pyrimidin-7-yl]-cyclobutylmethyl}-1H-imidazol-2-yl)-methanol

(R,S)-1-{cis-3-[4-Amino-5-(2-phenyl-chroman-7-yl)-pyrrolo[2,3-d]pyrimidin-7-yl]-cyclobutylmethyl}-1H-imidazole-2-carboxylic acid ethyl ester (Example 15, 30 mg, 0.05 mmol) was dissolved in THF (1.5 mL) and cooled to −78° C. under argon. At this temperature LiAlH₄ (2.0 M solution in THF, 71 mL, 0.16 mmol) was added dropwise and the reaction warmed to room temperature over 30 minutes. The reaction mixture was then submitted to aqueous workup and the crude material purified by flash chromatography (SiO₂; DCM/MeOH; gradient 0-10% MeOH). HPLC/MS (Method N) t_{R} 0.95 minute, M+H 507.2 (100%). HPLC_{A}: t_{R} 3.75 minutes.

### Example 19

### (R,S)—(Z)-3-[8-Amino-1-(2-phenyl-chroman-7-yl)-imidazo[1,5-a]pyrazin-3-yl]-1-methylcyclobutanol

The title compound is prepared in analogy to Example 1 from (R,S)-2-phenyl-7-(4,4,5,5-tetramethyl-[1,3,2]dioxaborolan-2-yl)chroman (Intermediate A) and 3-(8-amino-1-iodoimidazo[1,5a]pyrazin-3-yl)-1-methyl-cyclobutanol (WO2009/008992). HPLC/MS (Method N) t_{R} 0.92 minute, M+H 428.2 (100%). ¹H NMR (400 MHz, CD₃OD δ ppm 1.49 (s, 3H), 2.05-2.18 (m, 1H), 2.23-2.35 (m, 1H), 2.51-2.63 (m, 1H), 2.51-2.63 (m, 4H), 2.87 (ddd, 1H), 3.06 (dd, 1H), 3.43-3.53 (m, 1H), 5.18 (d, 1H), 6.97 (d, 2H), 7.12 (s, 1H), 7.27 (d, 1H), 7.31 (d, 1H), 7.37 (t, 2H), 7.42-7.48 (m, 3H).

### Example 20

### 7-[cis-3-(1,1-Dioxo-1-thiomorpholin-4-ylmethyl)-cyclobutyl]-5-(2-phenyl-4H-chromen-7-yl)-7H-pyrrolo[2,3-d]pyrimidin-4-ylamine

A stirred mixture of 5-bromo-7-[3-(1,1-dioxo-thiomorpholin-4-ylmethyl)-cyclobutyl]-7H-pyrrolo[2,3-d]pyrimidin-4-ylamine (intermediate O, 0.12 g, 0.30 mmol), 2-phenyl-7-(4,4,5,5-tetramethyl-[1,3,2]dioxaborolan-2-yl)-4H-chromene (Intermediate F, 0.15 g, 0.45 mmol), tetrakis(triphenylphosphine)palladium(0) (0.03 g, 0.02 mmol), sodium carbonate (0.06 g, 0.59 mmol) and potassium phosphate (0.13 g, 0.59 mmol) in DMF (2 ml) and water (0.11 ml) was heated under an argon atmosphere for 2 hours at 100° C. The cooled reaction mixture was partitioned between water and ethyl acetate, the organic layers were washed with brine, dried over sodium sulphate and evaporated to give the crude product which was purified by silica gel chromatography, eluting with a gradient of methanol in dichloromethane, to give the title compound as an orange/white solid. HPLC/MS (Method N) t_{R} 1.11 minutes, M+H 542.16 (100%).

### Example 21

### (R,S)-7-[cis-3-(1,1-Dioxo-1-thiomorpholin-4-ylmethyl)-cyclobutyl]-5-(2-phenyl-2H-chromen-7-yl)-7H-pyrrolo[2,3-d]pyrimidin-4-ylamine

A stirred mixture of 5-bromo-7-[cis-3-(1,1-dioxo-thiomorpholin-4-ylmethyl)-cyclobutyl]-7H-pyrrolo[2,3-d]pyrimidin-4-ylamine (Intermediate O, 0.10 g, 0.24 mmol), (R,S)-2-phenyl-7-(4,4,5,5-tetramethyl-[1,3,2]dioxaborolan-2-yl)-2H-chromene (Intermediate D, 0.121 g, 0.36 mmol), tetrakis(triphenylphosphine)palladium(0) (0.022 g, 0.019 mmol) and sodium carbonate (0.051 g, 0.483 mmol) in DMF (2 ml) and water (0.1 ml) was heated under an argon atmosphere for 1 hour at 100° C. The cooled reaction mixture was partitioned between water and dichloromethane, the organic layers were washed with brine, dried over sodium sulphate and evaporated to give the crude product which was purified by silica gel chromatography, eluting with a gradient of methanol in dichloromethane. The product containing fractions were combined, evaporated and further purified by Sepacore flash chromatography (8 g car-tridge). Product containing fractions were collected and evaporated to give the title compound as a white foam. HPLC/MS (Method N) t_{R} 1.10 minutes, M+H 542.5 (100%).

### Example 22

### (R,S)-{cis-3-[8-Amino-1-(2-phenyl-2H-chromen-7-yl)-imidazo[1,5-a]pyrazin-3-yl]-cyclobutyl}-methanol

The compound was obtained in analogy to the procedure described for Example 1 from [3-(8-amino-1-iodo-imidazol[1,5-a]pyrazin-3-yl-cyclobutyl]-methanol (prepared according to US20070129547 as 5:1 cis/trans mixture) and (R,S)-2-phenyl-7-(4,4,5,5-tetramethyl-[1,3,2]dioxaborolan-2-yl)-2H-chromene (Intermediate D). HPLC/MS (Method N) t_{R} 0.93 minutes, M+H 426.3 (100%). ¹H NMR (400 MHz, CD₃OD) δ ppm 2.18-2.31 (m, 2H), 2.47-2.71 (m, 3H), 3.55 (d, 2H), 3.75-3.88 (m, 1H), 5.94-6.02 (m, 2H), 6.68 (dd, 1H), 6.95 (d, 1H), 7.02 (s, 1H) 7.13 (d, 1H), 7.22 (d, 1H), 7.28-7.39 (m, 3H), 7.42-7.51 (m, 3H).

### Example 23

### (R,S)-{cis-3-[4-Amino-5-(2-phenyl-2H-chromen-7-yl)-pyrrolo[2,3-d]pyrimidin-7-yl]-cyclobutyl}-methanol

A stirred mixture of [cis-3-(4-amino-5-iodo-pyrrolo[2,3-d]pyrimidin-7-yl)-cyclobutyl]-methanol (Intermediate J, 0.13 g, 0.30 mmol), (R,S)-2-phenyl-7-(4,4,5,5-tetramethyl-[1,3,2]dioxaborolan-2-yl)-2H-chromene (Intermediate D, 0.12 g, 0.32 mmol), tetrakis(triphenylphosphine)palladium(0) (0.04 g, 0.03 mmol) and sodium carbonate (0.06 g, 0.60 mmol) in DMF (2 ml) and water (1 ml) was heated under an argon atmosphere for 20 hours at 80° C. The cooled reaction mixture was partitioned between water and dichloromethane, dried over sodium sulphate and evaporated to give the crude product which was purified by slica gel chromatography, eluting with a gradient of ethyl acetate in heptane, to give the title compound as a green/white foam. ¹H-NMR (d₆-DMSO, 600 MHz): b ppm 8.19 (s, 1H), 7.57 (s, 1H), 7.42 (d, 2H), 7.38 (d, 2H), 7.34-7.30 (m, 1H), 7.18 (d, 1H), 6.97 (d, 1H), 6.87 (s, 1H), 6.66 (d, 1H), 6.20 (s, br, 2H), 6.02 (s, 1H), 5.95-5.93 (m, 1H), 5.08-5.01 (m, 1H), 4.56 (t, 1H), 3.47-3.41 (m, 2H), 2.41-2.35 (m, 3H), 2.21-2.15 (m, 2H).

### Examples 24 and 25

### (R)-7-[cis-3-(1,1-Dioxo-thiomorpholin-4-ylmethyl)-cyclobutyl]-5-(2-phenyl-chroman-7-yl)-7H-pyrrolo[2,3-d]pyrimidin-4-ylamine and (S)-7-[cis-3-(1,1-dioxo-thiomorpholin-4-ylmethyl)-cyclobutyl]-5-(2-phenyl-chroman-7-yl)-7H-pyrrolo[2,3-d]pyrimidin-4-ylamine

(R,S)-7-[cis-3-(1,1-Dioxo-thiomorpholin-4-ylmethyl)-cyclobutyl]-5-(2-phenyl-chroman-7-yl)-7H-pyrrolo[2,3-d]pyrimidin-4-ylamine (Example 6) was separated by preparative chiral chromatography: Gilson preparative HPLC system; column: IA 250×10 mm, Chiralpak; eluent: 75% TBME/25% dichloromethane; injection volume 2 ml; flow 4 ml/min; detection UV 220 nm.

### Examples 26 and 27

### (S)-(E)-3-[4-Amino-5-(2-phenyl-chroman-7-yl)-pyrrolo[2,3-d]pyrimidin-7-yl]-1-hydroxymethyl-cyclobutanol and (R)-(E)-3-[4-amino-5-(2-phenyl-chroman-7-yl)-pyrrolo[2,3-d]pyrimidin-7-yl]-1-hydroxymethyl-cyclobutanol

Racemic (R,S)-(E)-3-[4-Amino-5-(2-phenyl-chroman-7-yl)-pyrrolo[2,3-d]pyrimidin-7-yl]-1-hydroxymethyl-cyclobutanol (Example 8, 96 mg, 0.217 mmol) was separated using a 20×210 mm Chiralpak IB column (Daicel Chemical Industries) and a Gilson chromatographic separator (isocratic mode: hexane/(EtOH/MeOH=1:1)=89:11; 12 mL/min; 3 mg portions per run) to give both enantiomers after evaporation of the solvent as colorless solids: First eluting enantiomer (Example 26): 42 mg (0.095 mmol, 43.8%), t^{R}=32.2 min. Second eluting enantiomer 2 (Example 27): 43 mg (0.097 mmol, 44.8%), t^{R}=35.2 min.

Alternatively, Example 26 (S)-(E)-3-[4-amino-5-(2-phenyl-chroman-7-yl)-pyrrolo[2,3-d]pyrimidin-7-yl]-1-hydroxymethyl-cyclobutanol can be prepared in the following manner. A mixture of (E)-3-(4-amino-5-iodo-pyrrolo[2,3-d]pyrimidin-7-yl)-1-hydroxymethyl-cyclobutanol (Intermediate Q, 2.25 g, 5.95 mmol), (S)-2-Phenyl-7-(4,4,5,5-tetramethyl-[1,3,2]dioxaborolan-2-yl)-chroman (Intermediate X, 2.00 g, 5.95 mmol), sodium carbonate (1.26 g, 11.90 mmol), potassium phosphate (2.14 g, 11.90 mmol), tetrakis-(triphenylphosphine)palladium(0) (0.55 g, 0.48 mmol), DMF (37.7 ml) and water (1.9 ml) was heated under an argon atmosphere for 3.5 hours at 100° C. The reaction mixture was partitioned between water and ethyl acetate, the organic layers washed with brine, dried over sodium sulphate and evaporated. Purification of the residue by normal phase chromatography, eluting with a gradient of methanol in DCM containing 0.5% concentrated ammonia solution, was followed by trituration in DCM to give the title compound as an off-white solid. ¹H NMR (400 MHz, DMSO) δ ppm 8.13 (s, 1H), 7.48 (s, 1H), 7.46 (d, 1H), 7.40 (t, 1H), 7.33 (t, 1H), 7.22 (d, 1H), 6.97 (d, 1H), 6.93 (s, 1H), 6.10 (s, br, 2H), 5.43-5.38 (m, 1H), 5.19 (d, 1H), 5.10 (s, 1H), 4.88 (t, 1H), 3.32 (d, 2H), 3.04-2.90 (m, 1H), 2.79-2.70 (m, 1H), 2.69-2.50 (m, 2H), 2.45-2.25 (m, 2H), 2.25-2.20 (m, 1H), 2.09-1.95 (m, 1H).

Alternatively, Example 27 (R)-(E)-3-[4-amino-5-(2-phenyl-chroman-7-yl)-pyrrolo[2,3-d]pyrimidin-7-yl]-1-hydroxymethyl-cyclobutanol can be prepared in the following manner. A mixture of (E)-3-(4-amino-5-iodo-pyrrolo[2,3-d]pyrimidin-7-yl)-1-hydroxymethyl-cyclobutanol (Intermediate Q, 65 mg, 0.180 mmol), (R)-2-Phenyl-7-(4,4,5,5-tetramethyl-[1,3,2]dioxaborolan-2-yl)-chroman (Intermediate Y, 67 mg, 0.180 mmol), sodium carbonate (38 mg, 0.361 mmol), potassium phosphate (63 mg, 0.361 mmol), tetrakis(triphenylphosphine)palladium(0) (10.4 mg, 0.009 mmol), DMF (1.2 ml) and water (0.06 ml) was heated under an argon atmosphere for 3 hours at 100° C. The reaction mixture was partitioned between water and DCM, the organic layers washed with brine, dried over sodium sulphate and evaporated. Purification of the residue by normal phase chromatography, eluting with a gradient of methanol in DCM containing 0.5% concentrated ammonia solution, was followed by trituration in DCM to give the title compound as an off-white solid. HPLC/MS (Method Z) t_{R} 0.84 minutes, M+H 433.2 (100%).

### Example 28

### (R,S)-4-{3-[4-amino-5-(2-phenyl-chroman-7-yl)-pyrrolo[2,3-d]pyrimidin-7-yl]-cyclobutylmethyl}-piperazin-2-one

2-Oxopiperazine (172 mg, 1.72 mmol) was added to a solution of toluene-4-sulphonic acid cis-3-[4-amino-5-(2-phenyl-chroman-7-yl)-pyrrolo[2,3-d]pyrimidin-7-yl]-cyclobutylmethyl ester (Intermediate T, 100 mg, 0.172 mmol) and triethylamine (0.24 ml, 1.72 mmol) in DMF (0.5 ml) and heated for 1 hour at 90° C., and then for 18 hours at 65° C. The cooled reaction mixture was then filtered and purified by preparative reversed phase chromatography (Method R). Product containing fractions were partitioned between DCM and aqueous sodium hydrogen carbonate solution, the organic layers dried over sodium sulphate and evaporated to give the title compound as an orange solid. HPLC/MS (Method Z) t_{R} 1.09 minutes, M+H 509.2 (100%).

### Example 29

### (E)-3-{4-Amino-5-[2-(tetrahydro-furan-2-yl)-chroman-7-yl]-pyrrolo[2,3-d]pyrimidin-7-yl}-1-hydroxymethyl-cyclobutanol

A mixture of (E)-3-(4-amino-5-iodo-pyrrolo[2,3-d]pyrimidin-7-yl)-1-hydroxymethyl-cyclobutanol (Intermediate Q, 250 mg, 0.69 mmol), 2-(tetrahydro-furan-2-yl)-7-(4,4,5,5-tetramethyl-[1,3,2]dioxaborolan-2-yl)-chroman (Intermediate U, 382 mg, 1.04 mmol), potassium phosphate (161 mg, 1.39 mmol), sodium carbonate (147 mg, 1.39 mmol), water (0.3 ml) and DMF (5 ml) was flushed with argon, tetrakis(triphenylphosphine)palladium(0) (65 mg, 0.06 mmol) added the sealed reaction vessel heated at 100° C. for 3 hours. The cooled reaction mixture was partitioned between water and DCM, extracted 2× with DCM, the organic layers dried over sodium sulphate and evaporated. Purification of the residue by normal phase chromatography, eluting with a gradient of methanol in DCM, gave the title compound as a mixture of the u- and l-diastereoisomers of the tetrahydrofuranyl chroman moiety. HPLC/MS (Method Z) t_{R} 0.83 and 0.86 minutes, M+H 437.1 and 437.1 (100%).

### Examples 30 and 31

### (I)-7-[cis-3-(1,1-Dioxo-1-thiomorpholin-4-ylmethyl)-cyclobutyl]-5-[2-(tetrahydro-furan-2-yl)-chroman-7-yl]-7H-pyrrolo[2,3-d]pyrimidin-4-ylamine and (u)-7-[3-(1,1-Dioxo-1-thiomorpholin-4-ylmethyl)-cyclobutyl]-5-[2-(tetrahydro-furan-2-yl)-chroman-7-yl]-7H-pyrrolo[2,3-d]pyrimidin-4-ylamine

A mixture of 5-bromo-7-[cis-3-(1,1-dioxo-thiomorpholin-4-ylmethyl)-cyclobutyl]-7′-1-pyrrolo[2,3-d]pyrimidin-4-ylamine (Intermediate O, 100 mg, 0.241 mmol), 2-(tetrahydro-furan-2-yl)-7-(4,4,5,5-tetramethyl-[1,3,2]dioxaborolan-2-yl)-chroman (Intermediate U, 133 mg, 0.362 mmol), potassium phosphate (56 mg, 0.483 mmol), sodium carbonate (51 mg, 0.483 mmol), water (0.1 ml) and DMF (1.9 ml) was flushed with argon, tetrakis-(triphenylphosphine)palladium(0) (65 mg, 0.06 mmol) added and the sealed reaction vessel heated at 100° C. for 1 hour. The cooled reaction mixture was partitioned between water and ethyl acetate, extracted 2× with ethyl acetate, the organic layers dried over sodium sulphate and evaporated. Purification of the residue by normal phase chromatography, eluting with a gradient of methanol in DCM, gave the title compound as a mixture of the u- and l-diastereoisomers of the tetrahydrofuranyl chroman moiety. HPLC/MS (Method Z) t_{R} 0.83 and 0.86 minutes, M+H 437.1 and 437.1 (100%).

Preparative reversed phase chromatography (Method R) gave the individual diastereoisomers, which were obtained as free bases following extraction from aqueous sodium bicarbonate solution with DCM, and were assigned based upon their ¹H nmr spectra in comparison with Intermediates V and W.

First eluting like-isomer as a white solid. ¹H NMR (400 MHz, DMSO) δ ppm 8.12 (s, 1H), 7.61 (s, 1H), 7.17 (d, 1H), 6.92 (d, 1H), 6.86 (s, 1H), 6.08 (s, br, 2H), 5.12-5.04 (m, 1H), 3.98-3.91 (m, 2H), 3.79-3.72 (m, 1H), 3.70-3.65 (m, 1H), 3.05 (s, br, 4H), 2.90 (s, br, 4H), 2.89-2.70 (m, 2H), 2.70-2.65 (m, 2H), 2.55-2.50 (m, 2H), 2.35-2.28 (m, 1H), 2.20-2.11 (m, 2H), 2.00-1.68 (m, 6H).

Second eluting unlike-isomer as a white solid. ¹H NMR (400 MHz, DMSO) δ ppm 8.12 (s, 1H), 7.61 (s, 1H), 7.17 (d, 1H), 6.92 (d, 1H), 6.86 (s, 1H), 6.10 (s, br, 2H), 5.12-5.04 (m, 1H), 3.97-3.89 (m, 2H), 3.80-3.73 (m, 1H), 3.69-3.63 (m, 1H), 3.05 (s, br, 4H), 2.89 (s, br, 4H), 2.88-2.71 (m, 2H), 2.71-2.67 (m, 2H), 2.55-2.50 (m, 2H), 2.35-2.29 (m, 1H), 2.22-2.13 (m, 2H), 2.13-2.09 (m, 1H), 2.03-1.95 (m, 1H), 1.92-1.81 (m, 3H), 1.69-1.60 (m, 1H).

### Examples 32 and 33

### (I)-(E)-3-{4-Amino-5-[2-(tetrahydro-furan-2-yl)-chroman-7-yl]-pyrrolo[2,3-d]pyrimidin-7-yl}-1-hydroxymethyl-cyclobutanol and (u)-(E)-3-{4-amino-5-[2-(tetrahydro-furan-2-yl)-chroman-7-yl]-pyrrolo[2,3-d]pyrimidin-7-yl}-1-hydroxymethyl-cyclobutanol

A mixture of (E)-3-(4-amino-5-iodo-pyrrolo[2,3-d]pyrimidin-7-yl)-1-hydroxymethyl-cyclobutanol (Intermediate Q, 160 mg, 0.444 mmol), 2-(tetrahydro-furan-2-yl)-7-(4,4,5,5-tetramethyl-[1,3,2]dioxaborolan-2-yl)-chroman (Intermediate U, 205 mg, 0.622 mmol), potassium phosphate (100 mg, 0.889 mmol), sodium carbonate (94 mg, 0.889 mmol), water (0.2 ml) and DMF (3.0 ml) was flushed with argon, tetrakis-(triphenylphosphine)palladium(0) (41 mg, 0.036 mmol) added and the sealed reaction vessel heated at 100° C. for 6.5 hour. The cooled reaction mixture was partitioned between water and DCM, extracted 2× with DCM, the organic layers washed with brine, dried over sodium sulphate and evaporated. Purification of the residue by normal phase chromatography, eluting with a gradient of methanol in DCM, gave the title compound as a mixture of the u- and l-diastereoisomers of the tetrahydrofuranyl chroman moiety. Preparative reversed phase chromatography (Method R) gave the individual diastereoisomers, which were obtained as free bases following extraction from aqueous sodium bicarbonate solution with DCM, and were assigned based upon their ¹H nmr spectra in comparison with Intermediates V and W.

First eluting like-isomer as a yellow foam. ¹H NMR (400 MHz, DMSO) δ ppm 8.13 (s, 1H), 7.46 (s, 1H), 7.16 (d, 1H), 6.90 (d, 1H), 6.83 (s, 1H), 6.10 (s, br, 2H), 5.44-5.36 (m, 1H), 5.10 (s, 1H), 4.90 (t, 1H), 4.00-3.89 (m, 2H), 3.78-3.74 (m, 1H), 3.71-3.67 (m, 1H), 3.33 (d, 2H), 2.89-2.72 (m, 2H), 2.66-2.60 (m, 2H), 2.32-2.24 (m, 2H), 2.00-1.71 (m, 6H).

Second eluting unlike-isomer as a white solid. ¹H NMR (400 MHz, DMSO) δ ppm 8.13 (s, 1H), 7.46 (s, 1H), 7.16 (d, 1H), 6.91 (d, 1H), 6.83 (s, 1H), 6.10 (s, br, 2H), 5.44-5.36 (m, 1H), 5.10 (s, 1H), 4.90 (t, 1H), 4.00-3.89 (m, 2H), 3.78-3.74 (m, 1H), 3.71-3.67 (m, 1H), 3.33 (d, 2H), 2.88-2.72 (m, 2H), 2.66-2.60 (m, 2H), 2.32-2.24 (m, 2H), 2.15-2.09 (m, 1H), 2.04-1.95 (m, 1H), 1.93-1.80 (m, 3H), 1.72-1.61 (m, 1H).

### Examples 34 and 35

### (R,R)-(E)-3-{4-Amino-5-[2-(tetrahydro-furan-2-yl)-chroman-7-yl]-pyrrolo[2,3-d]pyrimidin-7-yl}-1-hydroxymethyl-cyclobutanol and (S,S)-(E)-3-{4-amino-5-[2-(tetrahydro-furan-2-yl)-chroman-7-yl]-pyrrolo[2,3-d]pyrimidin-7-yl}-1-hydroxymethyl-cyclobutanol

The enantiomers of (I)-(E)-3-{4-amino-5-[2-(tetrahydro-furan-2-yl)-chroman-7-yl]-pyrrolo[2,3-d]pyrimidin-7-yl}-1-hydroxymethyl-cyclobutanol (Example 32, 25 mg, 0.057 mmol) were separated by preparative chiral HPLC: Chiralpak-OD column with 3:1:1 hexane:ethanol:methanol eluent.

First eluting enantiomer, t_{R} 31.79 minutes.
Second eluting enantiomer, t_{R} 49.06 minutes.

### Example 36

### (R,S)-8-{cis-3-[4-Amino-5-(2-phenyl-chroman-7-yl)-pyrrolo[2,3-d]pyrimidin-7-yl]-cyclobutylmethyl}-8-aza-bicyclo[3.2.1]octan-3-ol

A mixture of 8-[cis-3-(4-amino-5-bromo-pyrrolo[2,3-d]pyrimidin-7-yl)-cyclobutylmethyl]-8-aza-bicyclo[3.2.1]octan-(3-exo)-ol (Intermediate AB, 110 mg, 0.271 mmol), (R,S)-2-phenyl-7-(4,4,5,5-tetramethyl-[1,3,2]dioxaborolan-2-yl)chroman (Intermediate A, 100 mg, 0.298 mmol), potassium phosphate (115 mg, 0.541 mmol), sodium carbonate (57.4 mmol), tetrakis(triphenylphosphine)palladium(0) (16 mg, 0.014 mmol), water (0.2 ml) and DMF (2 ml) were heated under an argon atmosphere for 30 minutes at 100° C. The cooled reaction mixture was partitioned between water and ethyl acetate, extracted 2× with ethyl acetate, the organic layers washed with brine, dried over sodium sulphate and evaporated. Purification of the residue by normal phase chromatography, eluting with a gradient of methanol in DCM containing 1% concentrated aqueous ammonia solution, gave the title compound as a white foam. ¹H NMR (400 MHz, DMSO) δ ppm 1.45-1.55 (m, 2H), 1.70-2.25 (m, 11H), 2.40-2.55 (m, 4H), 2.70-3.10 (m, 4H), 3.75-3.85 (m, 1H), 4.23 (br. s., 1H), 5.00-5.20 (m, 2H), 6.10 (br. s, 2H), 6.90-7.01 (m, 2H), 7.19 (d, J=8.1 Hz, 1H), 7.29-7.50 (m, 5H), 7.60 (s, 1H), 8.09 (s, 1H)

### Example 37

### 1-{3-[4-Amino-5-(2-phenyl-chroman-7-yl)-pyrrolo[2,3-d]pyrimidin-7-yl]-cyclobutylmethyl}-piperidin-4-ol

The title compound was prepared in an analagous fashion to Example 36, but using 1-[cis-3-(4-amino-5-bromo-pyrrolo[2,3-d]pyrimidin-7-yl)-cyclobutylmethyl]-piperidin-4-ol (Intermediate AC) in place of Intermediate AB. The title compound was obtained as a white foam. ¹H NMR (400 MHz, DMSO) δ ppm 1.27-1.45 (m, 2H), 1.60-1.70 (m, 2H), 1.94-2.32 (m, 7H), 2.36-2.80 (m, 7H), 2.95-3.05 (m, 1H), 3.35 3.45 (m, 1H), 4.48 (d, J=3.9 Hz, 1H), 5.05 (t, J=8.0 Hz, 1H), 5.16 (d, J=7.8 Hz, 1H), 6.08 (br. s., 2H), 6.90-7.01 (m, 2H), 7.20 (d, J=8.2 Hz, 1H), 7.29-7.50 (m, 5H), 7.58 (s, 1H), 8.09 (s, 1H).

### Example 38

### 1-(4-{cis-3-[4-Amino-5-(2-phenyl-chroman-7-yl)-pyrrolo[2,3-d]pyrimidin-7-yl]-cyclobutyl}-piperazin-1-yl)-ethanone

The title compound was prepared in an analagous fashion to Example 36, but using 1-{4-[cis-3-(4-amino-5-bromo-pyrrolo[2,3-d]pyrimidin-7-yl)-cyclobutyl]-piperazin-1-yl}-ethanone (Intermediate AD) in place of Intermediate AB. The title compound was obtained as a white solid. ¹H NMR (400 MHz, DMSO) δ ppm 1.91-2.11 (m, 4H), 2.15-2.37 (m, 7H), 2.53-2.67 (m, 3H), 2.70-2.81 (m, 1H), 2.92-3.05 (m, 1H), 3.38-3.46 (m, 4H), 4.82-4.96 (m, 1H), 5.08-5.22 (m, 1H), 6.11 (br. s, 2H), 6.88-7.02 (m, 2H), 7.20 (d, J=8.2 Hz, 1H), 7.33 (t, J=7.0 Hz, 1H), 7.39 (t, J=7.6 Hz, 2H), 7.45 (d, J=6.6 Hz, 2H), 7.48 (s, 1 H), 8.11 (s, 1H).

### Example 39

### (1-{cis-3-[8-Amino-1-(2-phenyl-chroman-7-yl)-imidazo[1,5-a]pyrazin-3-yl]-cyclobutylmethyl}-1H-imidazol-2-yl)-methanol

A solution of lithium aluminium hydride in THF (1.7 M, 0.064 ml, 0.109 mmol) was added to a solution of (R,S)-1-{cis-3-[8-Amino-1-(2-phenyl-chroman-7-yl)-imidazo[1,5-a]pyrazin-3-yl]-cyclobutylmethyl}-1H-imidazole-2-carboxylic acid ethyl ester (Example 10, 20 mg, 0.036 mmol) in THF (2 ml) cooled at −78° C. The reaction mixture was stirred for 30 minutes at −78° C., then for 1 hour at room temperature and quenched by the addition of saturated aqueous ammonium chloride solution. The mixture was extracted 2× with DCM, the organic layers dried over sodium sulphate and evaporated. Purification of the residue by normal phase chromatography, eluting with a gradient of methanol in DCM, gave the title compound. HPLC/MS (Method Z) t_{R} 0.55 minutes, M+H 507.4 (10%).

### Example 40

### 7-[cis-3-(5-Methyl-tetrazol-1-ylmethyl)-cyclobutyl]-5-(2-phenyl-chroman-7-yl)-7H-pyrrolo[2,3-d]pyrimidin-4-ylamine

A mixture of toluene-4-sulphonic acid cis-3-[4-amino-5-(2-phenyl-chroman-7-yl)-pyrrolo[2,3-d]pyrimidin-7-yl]-cyclobutylmethyl ester (Intermediate T, 188 mg, 0.323 mmol), 5-methyltriazole (41 mg, 0.494 mmol), sodium hydride (60% dispersion in mineral oil, 20.7 mg, 0.517 mmol) and DMF (3 ml) was heated at 60° C. for 18 hours. The reaction mixture was partitioned between water and ethyl acetate, extracted 2× with ethyl acetate, the organic layers dried over sodium sulphate and evaporated. Purification of the residue by normal phase chromatography, eluting with a gradient of methanol in DCM, gve the title compound as a yellow solid. HPLC/MS (Method Z) t_{R} 1.15 minutes, M+H 493.2 (100%).

### Examples 41 and 42

### (Z)-3-K-Amino-5-(2-phenyl-chroman-7-yl)-pyrrolo[2,3-d]pyrimidin-7-yl]-1-hydroxymethyl-cyclobutanol

A mixture of Intermediates Q and R (0.3 g, 0.833 mmol), Intermediate A (0.345 g, 1.0 mmol), tetrakis triphenyl palladium (48 mg, 0.042 mmol), Na₂CO₃ (177 mg, 1.66 mmol), and K₃PO₄ (290 mg, 1.66 mmol) dissolved in H₂O (0.28 mL) and DMF (5.55 mL) were stirred at 100° C. for 1 h under Ar. After cooling down the reaction mixture to RT and adding H₂O (10 mL), the reaction mixture was extracted with AcOEt (30 mL, 4×). The combined organic phases were dried (MgSO₄), concentrated under reduced pressure, and purified by means of a Sepacore Control chromatographic separator (Büchi, Flawil, Switzerland) using a 40 g silica gel column (RediSept, Isco) (DCM->DCM/B=1:9 in 60 min; B=MeOH/25% aqueous NH₃=9:1, 40 mL/min) yielding the title compound (the Z-isomer) as a beige solid (70 mg).HPLC (Method B) t^{REt}=2.514 min. HPLC/MS t_{R}=0.94 min, M+H=443.1. (Method X). ¹H NMR (600 MHz, DMSO-d₆) δ ppm 8.41 (s, 1H), 7.97 (s, 1H), 7.45 (d, 2H), 7.39 (t, 2H), 7.32 (t, 1H), 7.24 (d, 1H), 7.03 (s, 1H), 6.99 (d, 1H), 6.20 (s/b, 2H), 5.25 (s, 1H), 5.20 (d, 1H), 4.86 (m, 2H), 3.35 (d, 2H), 3.00 (m, 1H), 2.27 (m, 1H), 2.70/2.36 (t/t, 2H/2H), 2.22/2.02 (m/m, 1H/1H).

Additionally, the corresponding (E)-isomer [(E)-3-[4-amino-5-(2-phenyl-chroman-7-yl)-pyrrolo[2,3-d]pyrimidin-7-yl]-1-hydroxymethyl-cyclobutanol] was isolated during the chromatographic separation (116 mg, beige solid). HPLC (method B) t^{Ret}=2.514 min. M+H=443.1. (method X). ¹H NMR (600 MHz, DMSO-d₆) δ ppm 8.12 (s, 1H), 7.48 (s, 1H), 7.47 (d, 2H), 7.41 (t, 2H), 7.34 (t, 1H), 7.21 (d, 1H), 6.97 (d, 1H), 6.94 (s, 1H), 6.15 (s/b, 2H), 5.36 (quintet, 1H), 5.19 (d, 1H), 5.15 (s, 1H), 4.85 (t, 1H), 3.32 (m, 2H), 3.00 (m, 1H), 2.76 (m, 1H), 2.69/2.27 (t/t, 2H/2H), 2.24/2.02 (m/m, 1H/1H).

A mixture of the S and R isomer mixture of (E)-3,4-amino-5-(2-phenyl-chroman-7-yl)-pyrrolo[2,3-d]pyrimidin-7-yl]-1-hydroxymethyl-cyclobutanol was separated by using a Chiral-pak AD 180×48 mm column, n-heptane/EtOH 60:40+0.1% TFA, 40 ml/min. In portions, 5-7 mg of material dissolved in MeOH were separated. By this method the first eluting enantiomer (Example 41) (>99% ee, 9.68 min retention time) was obtained followed by the second eluting enantiomer (Example 42) (>98% ee, 16.15 min redetection time).

### Example 43

### (E)-2-[4-Amino-5-(2-phenyl-chroman-7-yl)-pyrrolo[2,3-d]pyrimidin-7-yl]-5,7-dioxa-spiro[3.4]octan-6-one

[(E)-3-[4-amino-5-(2-phenyl-chroman-7-yl)-pyrrolo[2,3-d]pyrimidin-7-yl]-1-hydroxymethyl-cyclobutanol] (Examples 42 and 43, 20 mg, 0.045 mmol), DMAP (0.6 mg, 0.005 mmol), and 1,1′-carbonyldiimidazole (15.1 mg, 0.09 mmol) dissolved in DCM (0.8 mL) were stirred under Ar at RT for 3 h. After evaporating the solvent, the remaining beige residue (31 mg) was purified by silica gel chromatography according to the procedure described in Example 41. Step 1 using a 12 g column (RediSept) and a gradient from DCM->DCM/B=97:3 in 30 min yielding the title compound as white solid. HPLC (method B) t^{Ret}=2.772 min. M+H=469.3. (Method X). ¹H NMR (600 MHz, DMSO-d₆) δ ppm 8.15 (s, 1H), 7.63 (s, 1H), 7.46 (d, 2H), 7.41 (t, 2H), 7.34 (t, 1H), 7.22 (d, 1H), 6.97 (d, 1H), 6.94 (s, 1H), 6.15 (s/b, 2H), 5.36 (quintet, 1H), 5.19 (d, 1H), 4.65 (s, 2H), 3.09 (m, 2H), 2.99 (m, 3H), 2.76 (m, 1H), 2.24/2.02 (m/m, 1H/1H).

### Example 44

### (E)-2-[4-Amino-5-(2-phenyl-chroman-7-yl)-pyrrolo[2,3-d]pyrimidin-7-yl]-5-oxa-7-aza-spiro[3.4]octan-6-one

(E)-1-Aminomethyl-3-[4-amino-5-(2-phenyl-chroman-7-yl)-pyrrolo[2,3-d]pyrimidin-7-yl]-cyclobutanol (Intermediate AE, 60 mg, 0.129 mmol), and 1,1′-carbonyldiimidazole (25.9 mg, 0.155 mmol) dissolved in THF (1 mL) were stirred under Ar at RT for 6 h. After evaporating the solvent, H₂O (5 mL) was added and the reaction mixture was extracted with AcOEt (4 mL, 3×). The combined organic phases were extracted with brine (2 mL) and dried (MgSO₄).After evaporating the solvent the title compound remained as white solid. HPLC (method B) t^{Ret}=2.62 min. HPLC/MS t_{R}=1.02 min, M+H=468.2. ¹H NMR (600 MHz, DMSO-d₆) δ ppm 8.14 (s, 1H), 7.63 (s, 1H), 7.58 (s, 1H), 7.46 (d, 2H), 7.41 (t, 2H), 7.35 (t, 1H), 7.21 (d, 1H), 6.97 (d, 1H), 6.96 (s, 1H), 6.15 (s/b, 2H), 5.30 (quintet, 1H), 5.19 (d, 1H), 3.62 (d, 2H), 2.99 (m, 1H), 2.96 (m, 2H), 2.77 (t, 2H), 2.74 (m, 1H), 2.22/2.02 (m/m, 1H/1H).

### Example 45

### (E)-3-[4-Amino-5-(2-phenyl-chroman-7-yl)-pyrrolo[2,3-d]pyrimidin-7-yl]-1-(1,1-dioxothiomorpholin-4-ylmethyl)-cyclobutanol

Toluene-4-sulfonic acid (E)-3-[4-amino-5-(2-phenyl-chroman-7-yl)-pyrrolo[2,3-d]pyrimidin-7-yl]-1-hydroxy-cyclobutylmethyl ester (Step AE.2, 60 mg, 0.129 mmol), divinylsulfone (0.014 mL (0.142 mmol) were dissolved in DMF (1 mL) and a trace of silica gel was added. After stirring at RT for 18 h, the solvent was evaporated and the crude residue was purified by preparative TLC (2 20×20 cm silica gel plates, AcOEt/MeOH=85:15) to give the title compound as white solid. HPLC (method B) t^{Ret}=2.43 min. HPLC/MS t_{R}=1.03 min, M+H=560.3. ¹H NMR (600 MHz, DMSO-d₆) δ ppm 8.13 (s, 1H), 7.59 (s, 1H), 7.46 (d, 2H), 7.41 (t, 2H), 7.35 (t, 1H), 7.21 (d, 1H), 6.97 (d, 1H), 6.96 (s, 1H), 6.15 (s/b, 2H), 5.80 (s, 1H), 5.48 (quintet, 1H), 5.20 (d, 1H), 5.09 (s, 1H), 3.08 (s, 8H), 3.00/2.55 (m/m, 1H/1H), 2.73 (s, 2H), 2.55/2.30 (t/t, 2H/2H), 2.22/2.02 (m/m, 1H/1H).

### Example 46

### 4-{3-[4-Amino-5-(2-phenyl-chroman-7-yl)-pyrrolo[2,3-d]pyrimidin-7-yl]-cyclobutylmethyl}-1-methyl-piperazin-2-one

Intermediate A (36.7 mg, 0.109 mmol), 4-[cis-3-(4-amino-5-iodo-pyrrolo[2,3-d]pyrimidin-7-yl)-cyclobutylmethyl]-1-methyl-piperazin-2-one (Intermediate AF, 40 mg, 0.091 mmol), Na₂CO₃ (19.3 mg, 0.182 mmol), K₃PO₄ (38.6 mg, 0.182 mmol), and tetrakis triphenylphosphine palladium (5.25 mg, 4.54 mmol) dissolved in DMF 0.6 mL and H₂O (0.03 mL) and stirred at 100° C. under Ar for 100 min. After evaporation of the solvent, H₂O (5 mL) was added and the reaction mixture was extracted with AcOEt (10 mL, 3×). The combined organic phases were extracted with H₂O (2 mL) and brine (5 mL, 2×), dried (MgSO₄), and concentrated under reduced pressure. The resulting residue was purified by means of a Sepacore Control chromatographer (Büchi, Flawil, Switzerland) using RediSept silica gel column (12 g) (30 mL/min; DCM:10 min, DCM->DCM/MeOH/NH3 (90:10:1) in 30 min) yielding the title compound as white solid. HPLC (method B) t^{Ret}=2.396 min. HPLC/MS t_{R}=0.84 min, M+H=523.3. ¹H NMR (600 MHz, DMSO-d₆) δ ppm 8.13 (s, 1H), 7.63 (s, 1H), 7.46 (d, 2H), 7.41 (t, 2H), 7.35 (t, 1H), 7.21 (d, 1H), 6.97 (d, 1H), 6.96 (s, 1H), 6.15 (s/b, 2H), 5.19 (d, 1H), 5.12 (quintet, 1H), 3.26 (t, 2H), 3.08 (s, 8H), 3.02/2.55 (m/m, 1H/1H), 2.95 (s, 2H), 2.76 (s, 3H), 2.69 (t, 2H), 2.60 (d, 2H), 2.30 (m, 1H), 2.55/2.25 (m/m, 2H/2H), 2.22/2.02 (m/m, 1H/1H).

### Example 47

### (E)-3-[4-Amino-5-(2-phenyl-chroman-7-yl)-pyrrolo[2,3-d]pyrimidin-7-yl]-1-[1,2,4]triazol-4-ylmethylcyclobutanol

To the stirred mixture of (E)-1-aminomethyl-3-[4-amino-5-(2-phenyl-chroman-7-yl)-pyrrolo[2,3-d]pyrimidin-7-yl]-cyclobutanol (Intermediate AE. 60 mg, 0.135 mmol) and toluene (1.35 ml) was subsequently added N,N′-bis(dimethylaminomethylene)hydrazine (68 mg, 0.47 mmol) and pTsOH*H2O (2.5 mg, 0.013 mmol). The reaction mixture was stirred for 16 hours at 80° C. and then partitioned between saturated sodium bicarbonate solution and ethyl acetate. The combined organic layers were washed with brine, dried over sodium sulphate and evaporated. The residue was purified by flash chromatography, eluting with (DCM/MeOH/NH₃^{aq}, 200:20:1), to give the title compound as beige solid. HPLC/MS (Method Z) t_{R} 0.92 minutes, M+H 494.2. ¹H NMR (600 MHz, DMSO-d₆) δ ppm 8.45 (s, 2H), 8.13 (s, 1H), 7.65 (s, 1H), 7.47 (d, 2H), 7.43 (m, 1H), 7.24 (d, 1H), 6.98 (d, 2H), 6.16 (bs, 2H), 5.68 (s, 1H), 5.45 (m, 1H), 5.19 (m, 1H), 4.27 (s, 2H), 3.32 (s, 2H), 3.01 (m, 1H), 2.79 (m, 1H), 2.71 (m, 2H), 2.33 (m, 2H), 2.22 (m, 1H), 2.04 (m, 1H).

### Example 48

### cis-3-[4-Amino-5-(2-phenyl-chroman-7-yl)-pyrrolo[2,3-d]pyrimidin-7-yl]-cyclobutanol

To the stirred solution of 3-[4-amino-5-(2-phenyl-chroman-7-yl)-pyrrolo[2,3-d]pyrimidin-7-yl]-cyclobutanone (Intermediate AT, 130 mg, 0314 mol) and THF (3.14 ml) was added dropwise L-selectride in THF (1M, 0.627 mmol) at −78° C. The reaction mixture was stirred at −78° C. for 1 hour before quenching by the addition of NH4Cl 1M and extraction with ethyl acetate. The combined organic layers were washed with brine, dried over sodium sulphate and evaporated. The residue was purified by flash chromatography, eluting with (DCM/MeOH/NH₃^{aq}, 200:20:1), to give the title compound as beige foam. HPLC/MS (Method Z) t_{R} 1.04 minutes, M+H 413.1. ¹H NMR (600 MHz, DMSO-d₆) δ ppm 8.11 (s, 1H), 7.53 (s, 1H), 7.46 (d, 2H), 7.41 (t, 2H), 7.35 (t, 1H), 7.23 (d, 1H), 6.99 (d, 1H), 6.96 (s, 1H), 6.13 (bs, 2H), 5.25 (s, 1H), 5.19 (d, 1H), 4.72 (m, 1H), 4.01 (m, 1H), 3.0 (m, 1H), 2.78 (m, 3H), 2.35 (m, 2H), 2.20 (m, 1H), 2.05 (m, 1H).

### Example 49

### trans-3-[4-Amino-5-(2-phenyl-chroman-7-yl)-pyrrolo[2,3-d]pyrimidin-7-yl]-cyclobutanol

To the stirred solution of cis-3-{4-amino-5-[3-(7-oxa-bicyclo[2.2.1]hept-1-ylmethoxy)-phenyl]-pyrrolo[2,3-d]pyrimidin-7-yl}-cyclobutanol (Example 48; 108 mg, 0.25 mmol) and THF (2.5 mL) were added subsequently at room temperature, benzoic acid (61.2 mg, 0.50 mmol), triphenylphosphine (131 mg, 0.50 mmol) and diisopropyl azodicarboxylate (0.104 ml, 0.50 mmol). The reaction mixture was stirred for 50 min at room temperature and then partitioned between brine and EtOAc. The combined organic layers were dried (Na₂SO₄), filtered and concentrated. The residue was dissolved in MeOH (2.5 mL) and THF (0.5 mL) and potassium cabonate (173 mg, 1.25 mmol) were added. The mixture was stirred for 16 hours at room temperature and then partitioned between brine and EtOAc. The combined organic layers were dried (Na₂SO₄), filtered and concentrated. The residue was purified by flash chromatography (DCM/MeOH/NH₃^{aq}, 200:20:1) to afford the title compound as white foam: HPLC/MS (Method Z) t_{R} 0.92 minutes, M+H 413.3. ¹H NMR (600 MHz, DMSO-d₆) δ ppm 8.12 (s, 1H), 7.55 (s, 1H), 7.47 (d, 2H), 7.41 (t, 2H), 7.34 (m, 1H), 7.22 (d, 1H), 6.97 (m, 2H), 6.12 (bs, 2H), 5.38 (m, 1H), 5.21 (s, 1H), 5.17 (d, 1H), 4.45 (m, 1H), 3.00 (m, 1H), 2.79 (m, 1H), 2.70 (m, 2H), 2.37 (m, 2H), 2.23 (m, 1H), 2.04 (m, 1H).

### Example 50

### 5-(2-Ethoxymethyl-chroman-7-yl)-7-[cis-3-(1-oxo-thiomorpholin-4-ylmethyl)-cyclobutyl]-7H-pyrrolo[2,3-d]pyrimidin-4-ylamine

To the mixture of 5-iodo-7-[cis-3-(1-oxo-thiomorpholin-4-ylmethyl)-cyclobutyl]-7H-pyrrolo[2,3-d]pyrimidin-4-ylamine (Intermediate AU, 95 mg, 0.20 mmol), 2-ethoxymethyl-7-(4,4,5,5-tetramethyl-[1,3,2]dioxaborolan-2-yl)-chroman (Intermediate AG, 136 mg, 0.30 mmol), Na₂CO₃ (43 mg, 0.40 mmol), K₃PO₄ (85 mg, 0.40 mmol), DMF (2.0 ml) and water (0.10 ml) was added tetrakis triphenylphosphine palladium (11.56 mg, 0.01 mmol) under argon. The mixture was stirred for 1.5 hours at 100° C. and then partitioned between brine and EtOAc. The combined organic layers were dried (Na₂SO₄), filtered and concentrated. The residue was purified by reversed phase preparative chromatography (Method R) to afford the title compound as white foam: HPLC/MS (Method Z) t_{R} 0.64 minutes, M+H 510.4. ¹H NMR (600 MHz, DMSO-d₆) δ ppm 8.15 (bs, 1H), 7.61 (s, 1H), 7.17 (d, 1H), 6.93 (d, 1H), 6.88 (s, 1H), 6.11 (bs, 2H), 5.08 (m, 1H), 4.19 (m, 1H), 3.59 (m, 2H), 3.50 (m, 2H), 2.87 (m, 4H), 2.80-2.48 (m, 10H), 2.35 (m, 1H), 2.18 (m, 2H), 2.02 (m, 1H), 1.73 (m, 1H), 1.14 (t, 3H).

### Example 51

### (±)-N-(cis-4-(4-amino-5-(2-phenylchroman-7-yl)-7H-pyrrolo[2,3-d]pyrimidin-7-yl)cyclohexyl)cyclopropanecarboxamide

To a solution of 7-(cis-4-aminocyclohexyl)-5-(2-phenylchroman-7-yl)-7H-pyrrolo[2,3-d]pyrimidin-4-amine (Intermediate AK, 50 mg, 0.11 mmol) in DCM (2 mL), was added triethyl-lamine (0.2 mL), and cyclopropanecarbonyl chloride (17 mg, 0.165 mmol) respectively. The mixture was stirred for 30 min, and concentrated. The residue was purified by reverse phase preparative HPLC (Method 5), yielding the title compound. MS m/z 508.3 (M+H+) (Method M), ¹H NMR (CD₃OD-d4, 400 MHz): δ ppm 8.27 (s, 1H), 7.46-7.44 (m, 2H), 7.39-7.37 (m, 3H), 7.32-7.29 (m, 1H), 7.21 (d, 1H), 7.01-6.99 (m, 2H), 5.14 (dd, 1H), 4.69-4.64 (m, 1H), 4.17 (br, 1H), 3.08-3.01 (m, 1H), 2.85-2.81 (m, 1H), 2.28-2.03 (m, 4H), 1.87-1.71 (m, 7H), 0.88-0.84 (m, 2H), 0.78-0.73 (m, 2H).

### Example 52

### (±)-N-(cis-4-(4-amino-5-(2-phenylchroman-7-yl)-7H-pyrrolo[2,3-d]pyrimidin-7-yl)cyclohexyl)isobutyramide

To a mixture of isobutyric acid (15 mg, 0.165 mmol), triethylamine (0.2 mL), and HATU (50 mg, 0.13 mmol) in DCM (3 mL), was added 7-(cis-4-aminocyclohexyl)-5-(2-phenylchroman-7-yl)-7H-pyrrolo[2,3-d]pyrimidin-4-amine (Intermediate AK, 50 mg, 0.11 mmol) in DCM (0.5 mL). The mixture was stirred for 2 hours, and concentrated. The residue was purified by reverse phase preparative HPLC (Method S), yielding the title compound. MS m/z 510.3 (M+H+) (Method M). ¹H NMR (Acetone-d6, 400 MHz): δ ppm 8.14 (s, 1H), 7.97 (d, 1H), 7.44 (d, 2H), 7.37-7.35 (m, 3H), 7.32-7.28 (m, 1H), 7.21-7.19 (m, 1H), 7.00-6.98 (m, 2H), 5.14-5.11 (m, 1H), 4.68-4.62 (m, 1H), 4.12 (br, 1H), 3.08-3.01 (m, 1H), 2.83-2.79 (m, 1H), 2.63-2.56 (m, 1H), 2.26-2.24 (m, 1H), 2.17-2.05 (m, 3H), 1.92-1.83 (m, 6H), 1.13 (d, 6H).

### Example 53

### 7-(cis-3-(1,1-dioxo-thiomorpholin-4-ylmethyl)cyclobutyl)-5-(spiro[chroman-2,1′-cyclohexane]-7-yl)-7H-pyrrolo[2,3-d]pyrimidin-4-amine

The mixture of Intermediate O (87 mg, 0.21 mmol), Intermediate AP (69 mg, 0.21 mmol), Pd(PPh₃)₄ (12.1 mg, 0.01 mmol), and Na₂CO₃ (89 mg, 0.84 mmol) in a mixture of DMF (2.0 mL) and water (0.5 mL) was degassed and then heated in a microwave reactor at 120° C. for 10 min. The crude reaction mixture was purified by reverse phase preparative HPLC (Method S), yielding the title compound. MS m/z 536.3 (M+H+) (Method M).

### Example 54

### (±)-7-(cis-3-(1,1-dioxo-thiomorpholin-4-ylmethyl)cyclobutyl)-5-(2-(pyridin-2-yl)chroman-7-yl)-7H-pyrrolo[2,3-d]pyrimidin-4-amine

The mixture of Intermediate O (96.0 mg, 0.21 mmol), intermediate AQ (70 mg, 0.21 mmol), Pd(PPh₃)₄ (12.1 mg, 0.01 mmol), and Na₂CO₃ (89 mg, 0.84 mmol) in a mixture of DMF (2.0 mL) and water (0.5 mL) was degassed and then heated at 120° C. in the microwave reactor for 10 min. The crude reaction mixture was purified by reverse phase preparative HPLC (Method S), yielding the title compound. MS m/z 545.2 (M+H+) (Method M).

### Example 55

### (±)-N-(cis-4-(4-amino-5-(2-phenylchroman-7-yl)-7H-pyrrolo[2,3-d]pyrimidin-7-yl)cyclohexyl)acetamide

The title compound was prepared in a similar manner to Example 52 starting from 7-(cis-4-aminocyclohexyl)-5-(2-phenylchroman-7-yl)-7H-pyrrolo[2,3-d]pyrimidin-4-amine (Intermediate AK) and acetic acid. MS m/z 482.2 (M+H+) (Method M).

### Example 56

### (±)-(E)-3-(4-amino-5-(2-(pyridin-2-yl)chroman-7-yl)-7H-pyrrolo[2,3-d]pyrimidin-7-yl)-1-(hydroxymethyl)cyclobutanol

The mixture of Intermediates Q/R (53 mg, 0.15 mmol), intermediate AQ (50 mg, 0.15 mmol), Pd(PPh₃)₄ (8.6 mg, 0.01 mmol), and Na₂CO₃ (63 mg, 0.60 mmol) in a mixture of DMF (2.0 mL) and water (0.5 mL) was degassed and heated at 120° C. in the microwave reactor for 10 min. The crude reaction mixture was purified by reverse phase preparative HPLC (Method S), yielding the title compound. MS m/z 444.2 (M+H+) (Method M).

### Example 57

### (±)-4-((cis-3-(4-amino-5-(2-(pyridin-2-yl)chroman-7-yl)-7H-pyrrolo[2,3-d]pyrimidin-7-yl)cyclobutyl)methyl)-1-methylpiperazin-2-one

The mixture of Intermediate AF (26 mg, 0.08 mmol), intermediate AQ (34 mg, 0.08 mmol), Pd(PPh₃)₄ (4.4 mg, 0.008 mmol), and Na₂CO₃ (32 mg, 0.32 mmol) in a mixture of DMF (1.0 mL) and water (0.25 mL) was degassed and heated at 120° C. in the microwave reactor for 10 min. The crude reaction mixture was purified by reverse phase preparative HPLC (Method S), yielding the title compound. MS m/z 524.3 (M+H+) (Method M).

### Example 58

### (±)-N-((cis-3-(4-amino-5-(2-(pyridin-2-yl)chroman-7-yl)-7H-pyrrolo[2,3-d]pyrimidin-7-yl)cyclobutyl)methyl)acetamide

The mixture of Intermediate AR (19 mg, 0.05 mmol), Intermediate AQ (17 mg, 0.05 mmol), Pd(PPh₃)₄ (3.0 mg, 0.008 mmol), and Na₂CO₃ (21 mg, 0.2 mmol) in a mixture of DMF (1.0 mL) and water (0.25 mL) was degassed and heated at 120° C. for 10 min. in the microwave reactor. The crude reaction mixture was purified by reverse phase preparative HPLC (Method S), yielding the title compound. MS m/z 469.2 (M+H+) (Method M).

### Example 59

### (±)-N-(cis-4-(4-amino-5-(-2-(trans-tetrahydrofuran-2-yl)chroman-7-yl)-7H-pyrrolo[2,3-d]pyrimidin-7-yl)cyclohexyl)acetamide

The title compound was prepared in a similar manner to Intermediate AK, Step AK.1 starting from Intermediate AN and Intermediate AI. MS m/z 476.2 (M+H+) (Method M).

### Example 60

### (±)-N-(cis-4-(4-amino-5-(-2-(cis-tetrahydrofuran-2-yl)chroman-7-yl)-7H-pyrrolo[2,3-d]pyrimidin-7-yl)cyclohexyl)acetamide

The title compound was prepared in a similar manner to Intermediate AK, Step AK.1 starting from Intermediate AN and Intermediate AJ. MS m/z 476.2 (M+H+) (Method M). ¹H NMR (Acetone-d6, 400 MHz): δ ppm 8.37 (s, 1H), 7.57 (s, 1H), 7.29 (br, 1H), 7.20 (d, 1H), 7.00-6.97 (m, 1H), 6.90 (d, 1H), 4.89-4.83 (m, 1H), 4.18 (br, 1H), 4.03-4.01 (m, 2H), 3.86-3.72 (m, 3H), 2.95-2.80 (m, 3H), 2.32-2.20 (m, 2H), 2.01-1.82 (m, 7H), 1.88 (s, 3H).

### Example 61

### 7-[cis-3-((S,S)-2-oxo-2,6-thia-5-aza-bicyclo[2,2,1]hept-5-ylmethyl)-cyclobutyl]-5-(2-pyridin-2-yl-chroman-7-yl)-7H-pyrrolo[2,3-d]pyrimidin-4-ylamine

To a suspension of 7-(cis-3-((1S,4S)-2-thia-5-azabicyclo[2.2.1]heptan-5-ylmethyl)cyclobutyl)-5-iodo-7H-pyrrolo[2,3-d]pyrimidin-4-amine (Intermediate AS, 46 mg, 0.1 mmol) in MeCN (2 mL) was added oxone solution in water (0.1 M, 1 mL). After stirring at room temperature for 30 min, LCMS (Method M) showed complete conversion of the thioether in the starting material to the corresponding sulfoxide. The reaction mixture was dried using lyophylization to give an orange colored solid. This crude sulfoxide product was used in the next step without further purification. MS m/z 458.1 (M+H+) (Method M). The crude sulfoxide obtained in the previous step was reacted with Intermediate AQ in a similar manner as described for Example 58. The crude reaction mixture was purified by reverse phase preparative HPLC (Method S), yielding the title compound. MS m/z 541.2 (M+H+) (Method M).

### Example 62

### 7-[cis-3-((S,S)-2,2-Dioxo-2λ6-thia-5-aza-bicyclo[2,2,1]kept-5-ylmethyl)-cyclobutyl]-5-(2-pyridin-2-yl-chroman-7-yl)-7H-pyrrolo[2,3-d]pyrimidin-4-ylamine

The title compound was prepared in a manner similar to Example 58 via coupling Intermediate AO with Intermediate AQ. ¹H NMR (400 MHz, CD₃OD, TFA salt) δ 8.56 (m, 1H), 8.13 (s, 1H), 7.88 (dt, J=1.6, 7.6 Hz, 1H), 7.64 (d, J=7.6 Hz, 1H), 7.42 (s, 1H), 7.38 (ddd, J=0.8, 4.8, 7.6 Hz, 1H), 7.22 (d, J=7.6 Hz, 1H), 7.06 (d, J=1.2 Hz, 1H), 7.03 (dd, J=1.6, 8.0 Hz, 1H), 5.21 (dd, J=1.8, 9.6 Hz, 1H), 5.10 (m, 1H), 3.81 (t, J=2.8 Hz, 1H), 3.57 (m, 1H), 3.38 (m, 1H), 3.26 (d, J=11.6 Hz, 1H), 3.13 (dd, J=4.4, 11.2 Hz, 1H), 3.05 (m, 1H), 2.94 (dd, J=3.2, 12.8 Hz, 1H), 2.83 (m, 3H), 2.69 (m, 2H), 2.46-2.30 (m, 4H), 2.24 (m, 2H), 2.13 (m, 1H); MS m/z 557.2 (M+H+) (Method M).

### Example 63

### (±)-7-(cis-3-(1-oxo-thiomorpholin-4-ylmethyl)cyclobutyl)-5-(2-(pyridin-2-yl)chroman-7-yl)-7H-pyrrolo[2,3-d]pyrimidin-4-amine

The title compound was prepared in a manner similar as described for Intermediate AS and Example 61, using appropriate starting materials. ¹H NMR (400 MHz, CD₃OD, TFA salt) δ 8.55 (d, J=4.8 Hz, 1H), 8.13 (s, 1H), 7.87 (dt, J=1.6, 7.6 Hz, 1H), 7.62 (d, J=7.6 Hz, 1H), 7.41 (s, 1H), 7.37 (dd, J=5.6, 7.2 Hz, 1H), 7.20 (d, J=8.0 Hz, 1H), 7.03 (m, 2H), 5.20 (dd, J=2.4, 9.6 Hz, 1H), 5.10 (m, 1H), 3.07-2.94 (m, 5H), 2.89 (m, 2H), 2.78 (m, 3H), 2.73-2.66 (m, 4H), 2.46-2.37 (m, 2H), 2.41 (m, 2H), 2.12 (m, 1H); MS m/z 529.2 (M+H+) (Method M).

### Example 64

### (±)-7-[cis-4-(1,1-dioxo-1λ6-thiomorpholin-4-yl)-cyclohexyl]-5-[2-trans-(tetrahydro-furan-2-yl)chroman-7-yl]-7H-pyrrolo[2,3-d]pyrimidin-4-ylamine

The title compound was prepared in a similar manner to Intermediate AK, Step AK.1 starting from Intermediate AM and Intermediate AI. MS m/z 552.2 (M+H+) (Method M). ¹H NMR (CDCl₃, 400 MHz): δ ppm 10.27 (s, 1H), 8.22 (s, 1H), 7.14 (d, 1H), 6.83-6.81 (m, 2H), 6.08 (s, 1H), 4.90 (s, 1H), 4.02-3.83 (m, 4H), 3.81 (m, 4H), 3.47-3.38 (m, 5H), 2.87-2.83 (m, 2H), 2.50-2.46 (m, 2H), 2.18-1.73 (m, 11H).

### Example 65

### (±)-7-[cis-4-(1,1-dioxo-1λ6-thiomorpholin-4-yl)-cyclohexyl]-5-[2-cis-(tetrahydro-furan-2-yl)chroman-7-yl]-7H-pyrrolo[2,3-d]pyrimidin-4-ylamine

The title compound was prepared in a similar manner to Intermediate AK, Step AK.1 starting from Intermediate AM and Intermediate AJ. MS m/z 552.2 (M+H+) (Method M). ¹H NMR (CDCl3, 400 MHz): δ ppm 8.27 (s, 1H), 7.09 (d, 1H), 7.03-7.01 (m, 2H), 6.93 (dd, 1H), 5.23 (s, 1H), 4.82-4.77 (m 1H), 3.98-3.81 (m, 4H), 3.13-3.05 (m, 7H), 2.92-2.78 (m, 2H), 2.67 (br, 1H), 2.12-1.68 (m, 14H).

### Example 66

### 7-[cis-3-((S,S)-2,2-dioxo-2λ6-thia-5-aza-bicyclo[2,2,1]hept-5-ylmethyl)-cyclobutyl]-5-[2-trans-(tetrahydro-furan-2-yl-chroman-7-yl)-7H-pyrrolo[2,3-d]pyrimidin-4-ylamine

The title compound was prepared in a similar manner to Intermediate AK, Step AK.1 starting from Intermediate AO and Intermediate AI. MS m/z 550.2 (M+H+) (Method M). ¹H NMR (CD₃OD-d4, 400 MHz): δ ppm 8.12 (s, 1H), 7.38 (s, 1H) 7.15 (d, 1H), 6.95 (dd, 1H), 6.89 (d, 1H), 5.13-5.04 (m, 1H), 4.04-3.96 (m, 2H), 3.94-3.89 (m, 1H), 3.83-3.78 (m, 2H), 3.56 (br, 1H), 3.37 (dd, 1H), 3.26 (d, 1H), 3.13 (dd, 1H), 2.94 (dd, 1H), 2.90-2.78 (m, 3H), 2.70-2.64 (m, 2H0, 2.46-2.32 (m, 3H), 2.27-2.16 (m, 3H), 2.10-2.07 (m, 1H), 1.99-1.94 (m, 2H), 1.80-1.70 (m, 1H).

### Example 67

### 7-[cis-3-((S,S)-2,2-dioxo-2λ6-thia-5-aza-bicyclo[2,2,1]hept-5-ylmethyl)-cyclobutyl]-5-[2-cis-(tetrahydro-furan-2-yl-chroman-7-yl)-7H-pyrrolo[2,3-d]pyrimidin-4-ylamine

The title compound was prepared in a similar manner to Intermediate AK, Step AK.1 starting from Intermediate AO and Intermediate AJ. MS m/z 550.2 (M+H+) (Method M). ¹H NMR (CD₃OD-d4, 400 MHz): δ ppm 8.12 (s, 1H), 7.39 (s, 1H), 7.16 (d, 1H), 6.96 (dd, 1H), 6.93 (d, 1H), 5.13-5.05 (m, 1H), 4.05-3.81 (m, 5H), 3.57 (br, 1H), 3.36 (dd, 1H), 3.26 (d, 1H), 3.14 (dd, 1H), 2.95 (dd, 1H), 2.91-2.81 (m, 3H), 2.71-2.63 (m, 2H), 2.46-2.31 (m, 3H), 2.28-2.18 (m, 2H), 2.03-1.83 (m, 5H).

### Examples 68 and 69

### 7-[cis-3-((S,S)-2,2-dioxo-2λ6-thia-5-aza-bicyclo[2,2,1]hept-5-ylmethyl)-cyclobutyl]-5-[(R,R)-2-(tetrahydro-furan-2-yl-chroman-7-yl)-7H-pyrrolo[2,3-d]pyrimidin-4-ylamine and 7-[cis-3-((S,S)-2,2-dioxo-2λ6-thia-5-aza-bicyclo[2,2,1]hept-5-ylmethyl)-cyclobutyl]-5-[(S,S)-2-(tetrahydro-furan-2-yl-chroman-7-yl)-7H-pyrrolo[2,3-d]pyrimidin-4-ylamine

Example 66 was separated into its pure enantiomers via chiral Preparatory HPLC (Column: 20×250 mm ChiralPak IA; Conditions: 24 mL/min flow rate, 60/30/10 Hexane/CHCl₃/EtOH with 0.1% DEA as modifier; Run Time: 15 minutes). Analytical chiral HPLC retention times: 13.35 min. (Example 68) and 16.79 min (Example 69) under these analytical chiral HPLC conditions: Column: 4.6×250 mm ChiralPak IA; 1 mL/min flow rate, 60/30/10 Hexane/CHCl₃/EtOH). Example 68 as the first eluting enanantimer. MS m/z 542.3 (M+H+) (Method M). Example 69 as the second eluting enantiomer. MS m/z 542.3 (M+H+) (Method M). ¹H NMR data were identical for Example 66, 68 and 69.

### Example 70

### (±)-4-(cis-4-(4-amino-5-(2-phenylchroman-7-yl)-7H-pyrrolo[2,3-d]pyrimidin-7-yl)cyclohexyl)-1-methylpiperazin-2-one

To a mixture of 4-(4-amino-5-(2-phenylchroman-7-yl)-7H-pyrrolo[2,3-d]pyrimidin-7-yl)cyclohexanone (Intermediate AL, 100 mg, 0.23 mmol), 1-methylpiperazin-2-one (137 mg, 0.92 mmol), diisopropylethylamine (1.84 mmol, 327 uL) in dichloroethane (3 mL), was added sodium triacetoxyborohydride (195 mg, 0.92 mmol). The mixture was stirred at room temperature for 2 h and then concentrated. The resulting residue was purified to a mixture of the cis and trans isomers via reverse phase preparative HPLC (Method S). Preparative silica TLC, eluting with 10% MeOH in DCM with 0.1N ammonia, was used to isolate the cis (lower band) isomer. MS m/z 537.3 (M+H+) (Method M).

IVa Cellular IGF-1R and InsR assays

Compound-mediated inhibition of IGF1R and INSR phosphorylation in Hek293 cells transduced with the corresponding receptors is assessed in a capture ELISA format using the MSD (Meso Scale Discovery) platform. Briefly, 30′000 cells washed and diluted in starvation medium (DMEM high glucose supplemented with 0.1% BSA) are seeded in 90 μL per well into 96-well plates pre-coated with poly-D-lysine (0.1 mg/mL in PBS/O). After 24 h incubation at 37° C. and 5% CO2, dose-response effects are determined with 3-fold serial compound dilutions, starting at 10 μM. The final vehicle concentration is 0.1% DMSO in all wells. Following pre-incubation with compounds for 1 h, receptor phosphorylation is triggered by a 10 min exposure to 1.0 ng/μL IGF for Hek293-IGF1R cells, and 5.0 ng/μL insulin for Hek293-InsR cells. Cell lysis is achieved by addition of 80 μL MSD lysis buffer per aspirated well, incubation on ice for 20 min, and a freeze-thaw cycle. Target phosphorylation is then assessed by transferring volumes corresponding to approx. 6 μg Hek293-IGF1R or 0.6 μg Hek293-lnsR lysates to MSD assay plates pre-coated with total-IGF1R or total-lnsR Abs, respectively. After incubation for 2 h at rt, wells are exposed for 1 hr to a rabbit monoclonal antibody (CST #3024, 1:1000) detecting pIGF1R(Tyr1135/1136) as well as pINSR(Tyr1150/1151). Immune complexes are detected by a SULFO-Tag™-coupled anti-rabbit IgG antibody in the presence of 150 μL MSD read-buffer. Light emission at 620 nm triggered by application of electric current is recorded on a MSD Sectorlmager 6000. Acquired raw data (mean Ru-ECL units) are processed in an Excel analysis template. The plate blank (MSD lysis buffer) is subtracted from all data points. The effect of a particular test compound concentration on receptor phosphorylation is expressed relative to the window defined by ligand-stimulated vs unstimulated control cells (set as 100%). IC50 values [nM] are determined using 4-parametric curve-fitting (XLfit software, V4.3.2).

### IVb Enzymatic Assays

Two methods were used to analyze phosphorylated peptides and proteins produced by the listed tyrosine and serine/threonine-specific protein kinases: either using a filter-binding assay (FB), or using a flashplate assay (FP). The activities of protein kinases were assayed in the presence or absence of inhibitors, by measuring the incorporation of ³³P from [γ³³P]ATP into appropriate substrates.

### Filter-Binding Assay

96-well polypropylene microplates were used to assay the activity in the FB mode. To determine the inhibitory activity of compounds 10 μL of compound dilutions were pipetted into 96-well plates followed by the addition of 10 μL of assay mix and 10 μL of individual enzymes. With the addition of the enzymes the reactions were initiated and continued at RT. The reactions were stopped by the addition of 50 μL of a 125 mM EDTA solution pH 8.0. The final concentration of DMSO in the enzyme assays was 1%.

### Flashplate Assay

Flashplates are available as 96-well standard (STFPs) or as streptavidin-(SAFPs) or nickel coated FPs (NiFPs) from Perkin Elmer. STFPs are 96-well polystyrene microplates in which the interior of each well is permanently coated with a thin layer of polystyrene-based scintillant. Streptavidin flashplates (SAFP) are 96 or 384-well STFPs coated with streptavidin. SAFPs are suitable for a wide variety of assay applications which utilize biotinylated capture molecules. NiFP or nickel chelate flashplates are 96- or 384-well STFPs coated with nickel chelate. NiFPs are designed for in-plate, radiometric assays which utilize 4- or 6-histidine tagged proteins and peptides.

All kinase assays were performed in STFPs for 60 mins at RT and stopped with 50 μL of 0.5% H₃PO₄ except PKA which were carried out in polypropylene 96- and 384-well plates, respectively. PKA assays were stopped with 50 μL of 125 mM EDTA (pH 8.0) and 50 μL were transferred to either SAFPs or NIFPs to capture the biotinylated or histidine tagged peptides phosphorylated by PKA (SAFP) or by NiFPs. All wells were then washed three times with 200 μL of 0.5% H₃PO₄ and the plates were dried at room temperature. The plates were sealed and counted in a microplate scintillation counter (TopCount NXT, TopCount NXT HTS). The final concentration of DMSO in the enzyme assays was 1%.

Format and Incubation (min) of the Protein Kinase Assays

| | Filter-binding, | Flashplate, | Flashplate, |
|---|---|---|---|
| Enzyme | 96 wells | 96 wells | 384 wells |
| c-Abl-T315l | 10 | 30 | 30 |
| CDK2/A (Bt-P) | 30 | 30 | NT |
| c-Kit | 10 | 30 | 30 |
| c-Met | 15 | 30 | 30 |
| EphA4 | 10 | NT | NT |
| FGFR-4 | 10 | NT | NT |
| HER-2 | 15 | 30 | 30 |
| IGF-1R | 20 | 30 | 30 |
| Ins-R | 20 | 30 | 30 |
| JAK-2 | 10 | 30 | 30 |
| KDR | 10 | 30 | 30 |
| P38 | 10 | NT | NT |
| PKA (Bt-P) | 45 | 60 | 60 |
| | | (SAFP) | (SAFP) |
| Ret | 10 | 30 | 30 |

NT: not tested

Test results obtained using the above describe methods are summarized in the tables below.

| Ex. | Structure | 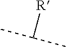 | 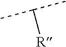 | IGF-1R IC₅₀ (nM) |
|---|---|---|---|---|
| 1 | 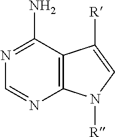 | 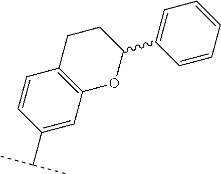 | 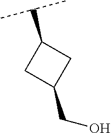 | 11-22 |
| 2 | 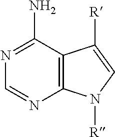 | 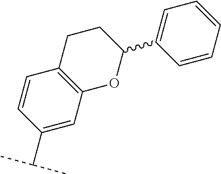 | 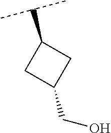 | 30-103 |
| 3 | 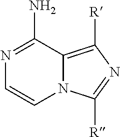 | 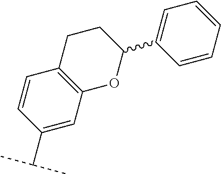 | 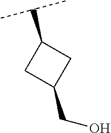 | 76 |
| 4 | 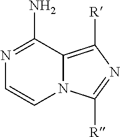 | 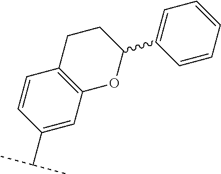 | 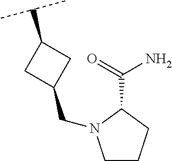 | 346 |
| 5 | 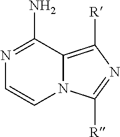 | 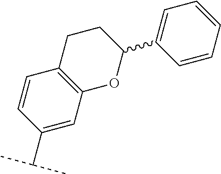 | 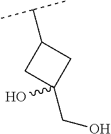 | 100 |
| 6 | 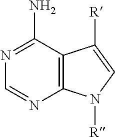 | 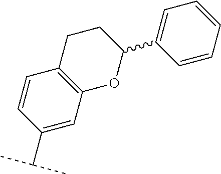 | 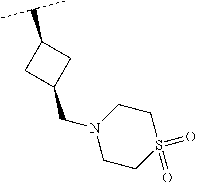 | 3 |
| 7 | 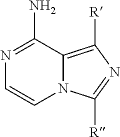 | 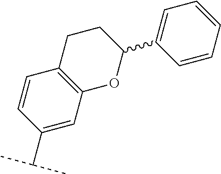 | 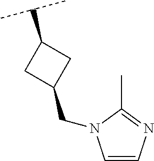 | 26 |
| 8 | 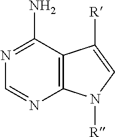 | 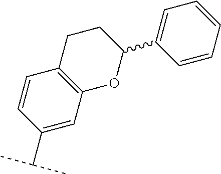 | 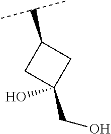 | 95 |
| 9 | 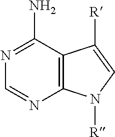 | 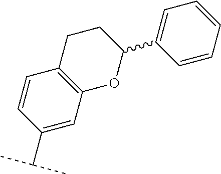 | 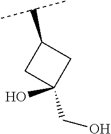 | 158 |
| 10 | 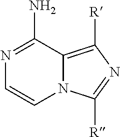 | 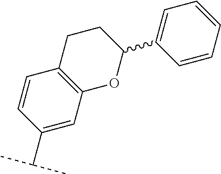 | 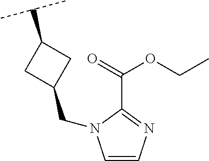 | |
| 11 | 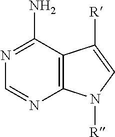 | 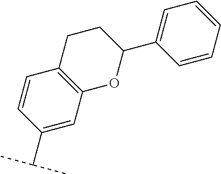 | 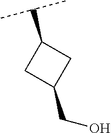 | 19 |
| 12 | 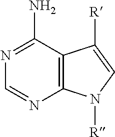 | 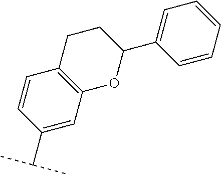 | 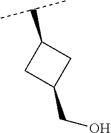 | 55 |
| 13 | 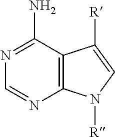 | 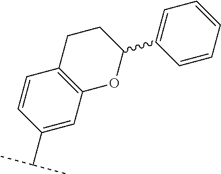 | 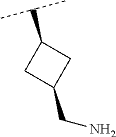 | 39 |
| 14 | 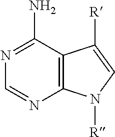 | 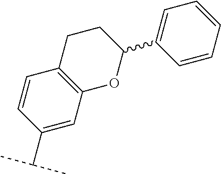 | 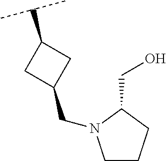 | <5 |
| 15 | 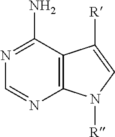 | 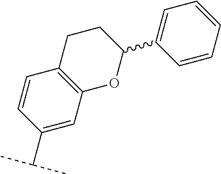 | 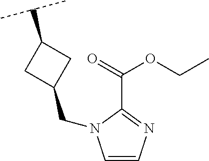 | |
| 16 | 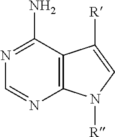 | 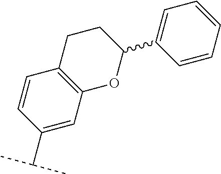 | 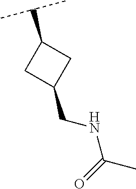 | 22 |
| 17 | 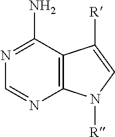 | 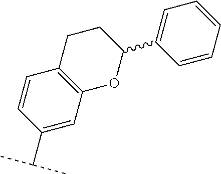 | 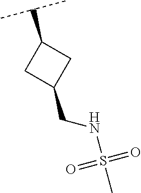 | 24 |
| 18 | 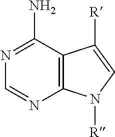 | 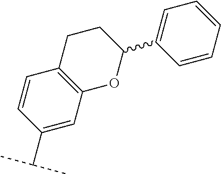 | 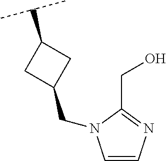 | 14 |
| 19 | 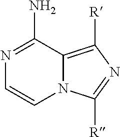 | 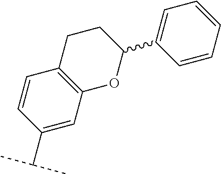 | 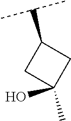 | 158 |
| 20 | 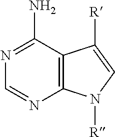 | 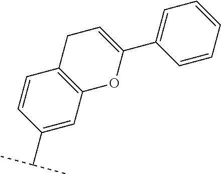 | 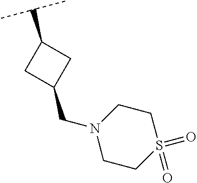 | 179 |
| 21 | 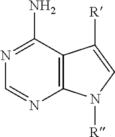 | 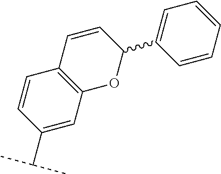 | 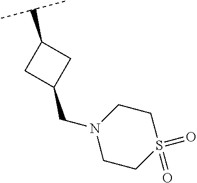 | 9 |
| 22 | 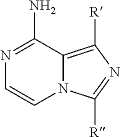 | 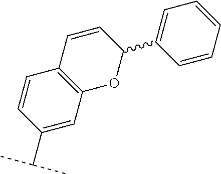 | 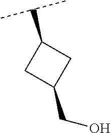 | 179 |
| 23 | 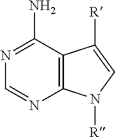 | 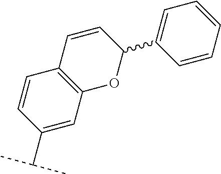 | 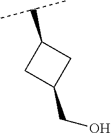 | 12 |
| 24 | 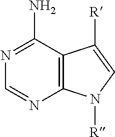 | 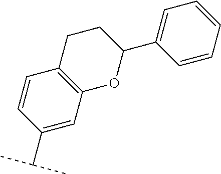 | 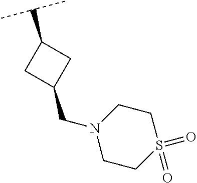 | 8 |
| 25 | 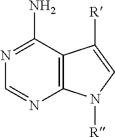 | 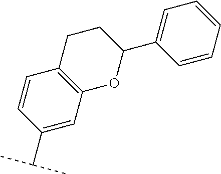 | 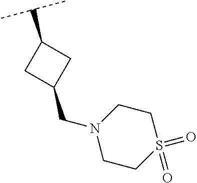 | 9 |
| 26 | 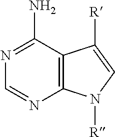 | 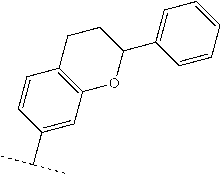 | 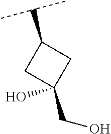 | 27-106 |
| 27 | 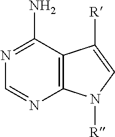 | 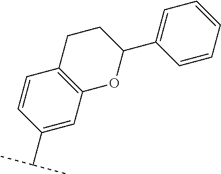 | 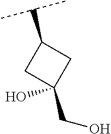 | 31-112 |
| 28 | 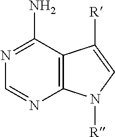 | 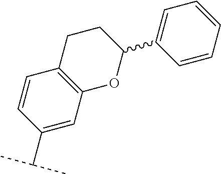 | 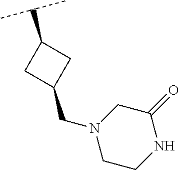 | 34 |
| 29 | 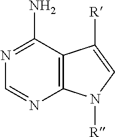 | 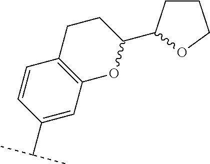 | 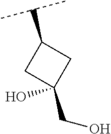 | 50 |
| 30 | 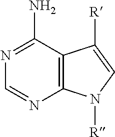 | 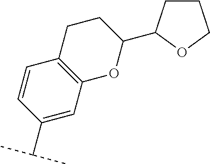 | 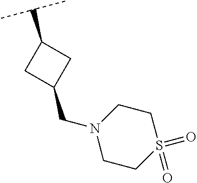 | 4-5 |
| 31 | 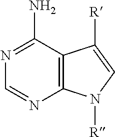 | 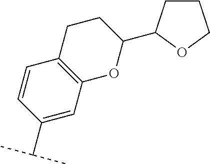 | 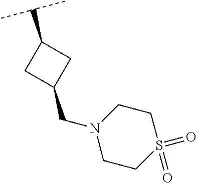 | 7 |
| 32 | 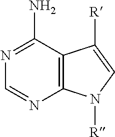 | 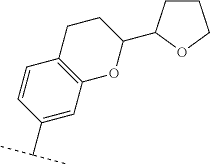 | 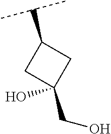 | 183 |
| 33 | 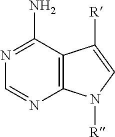 | 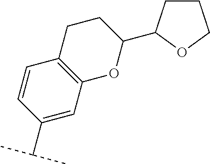 | 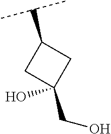 | 395 |
| 34 | 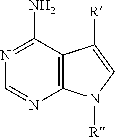 | 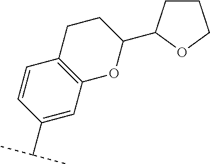 | 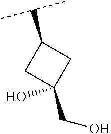 | 533 |
| 35 | 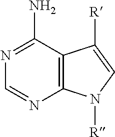 | 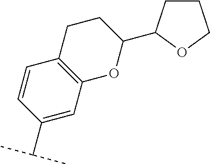 | 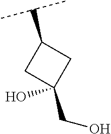 | 55 |
| 36 | 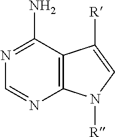 | 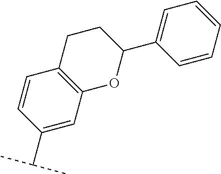 | 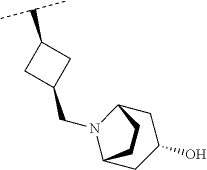 | 87 |
| 37 | 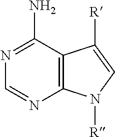 | 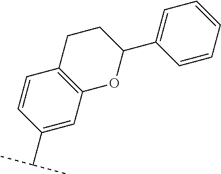 | 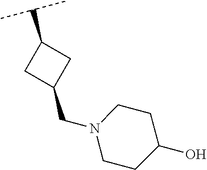 | 46 |
| 38 | 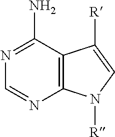 | 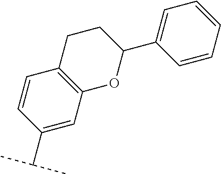 | 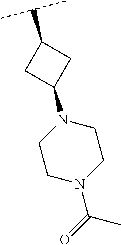 | 11 |
| 39 | 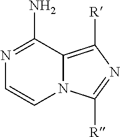 | 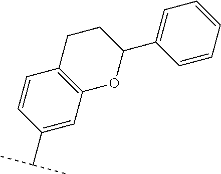 | 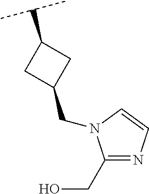 | 218 |
| 40 | 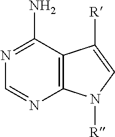 | 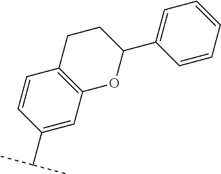 | 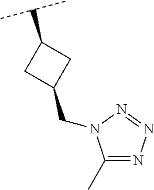 | |
| 41 | 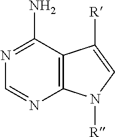 | 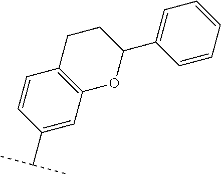 | 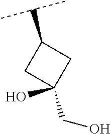 | 44-143 |
| 42 | 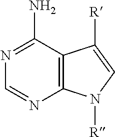 | 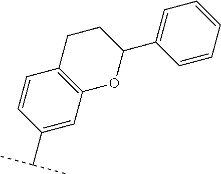 | 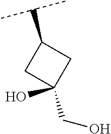 | 272-338 |
| 43 | 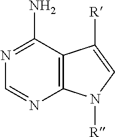 | 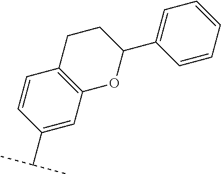 | 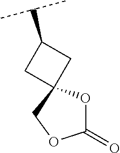 | 76 |
| 44 | 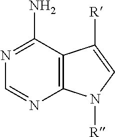 | 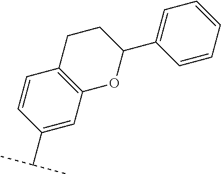 | 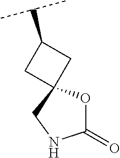 | 16 |
| 45 | 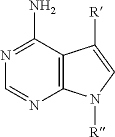 | 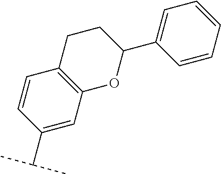 | 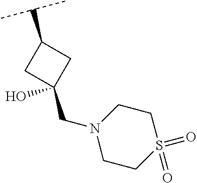 | <5 |
| 46 | 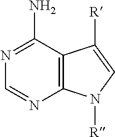 | 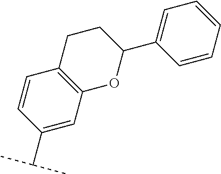 | 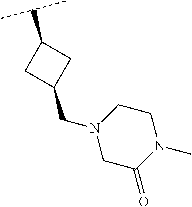 | 49 |
| 47 | 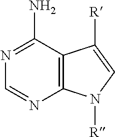 | 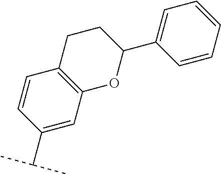 | 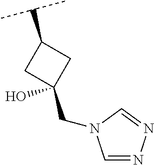 | 525 |
| 48 | 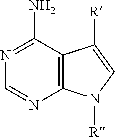 | 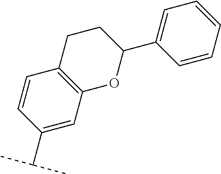 | 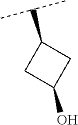 | 88 |
| 49 | 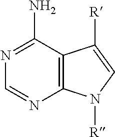 | 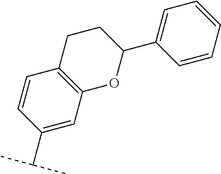 | 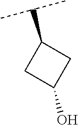 | 96 |
| 50 | 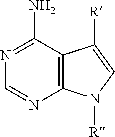 | 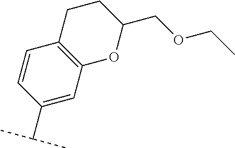 | 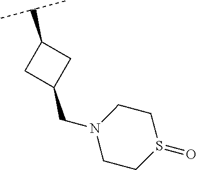 | 36 |
| 51 | 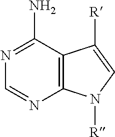 | 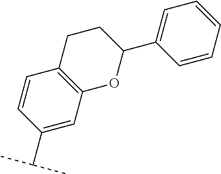 | 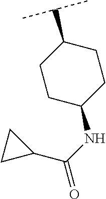 | 136 |
| 52 | 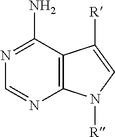 | 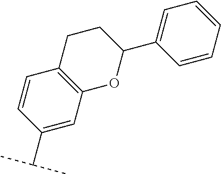 | 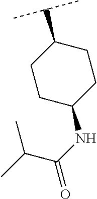 | 99 |
| 53 | 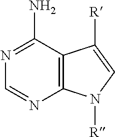 | 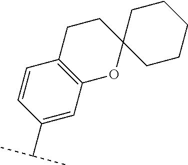 | 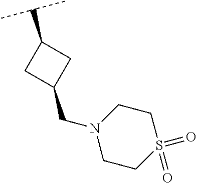 | 202 |
| 54 | 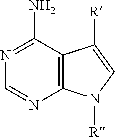 | 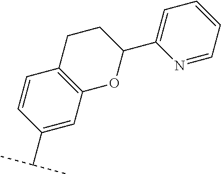 | 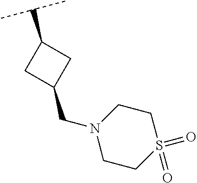 | 45 |
| 55 | 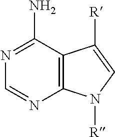 | 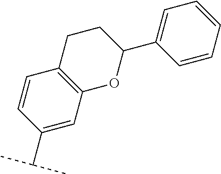 | 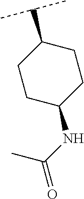 | 108 |
| 56 | 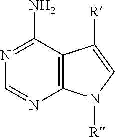 | 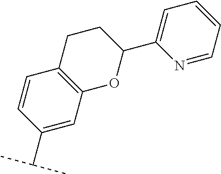 | 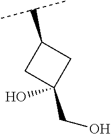 | 548 |
| 57 | 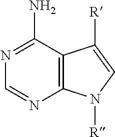 | 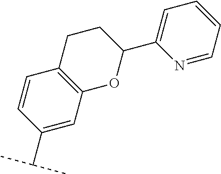 | 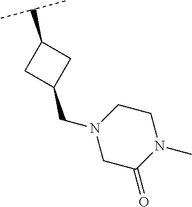 | |
| 58 | 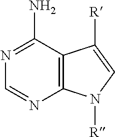 | 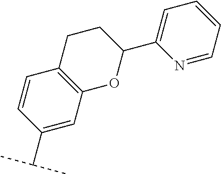 | 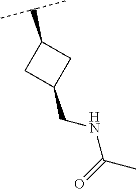 | |
| 59 | 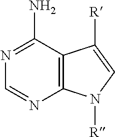 | 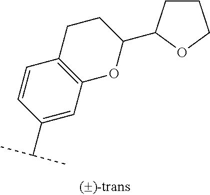 | 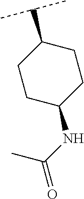 | |
| 60 | 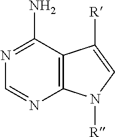 | 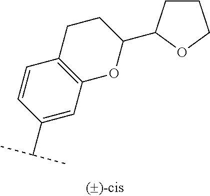 | 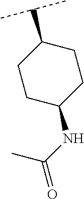 | |
| 61 | 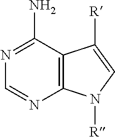 | 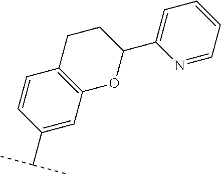 | 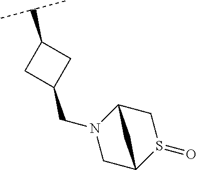 | |
| 62 | 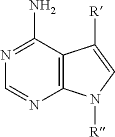 | 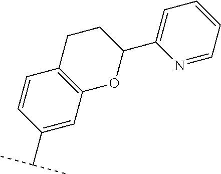 | 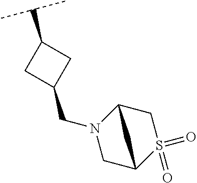 | 69 |
| 63 | 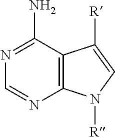 | 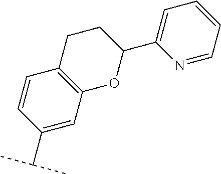 | 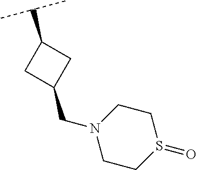 | 109 |
| 64 | 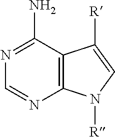 | 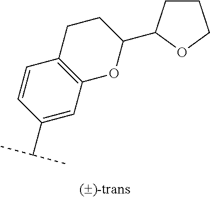 | 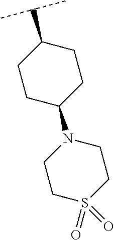 | 59 |
| 65 | 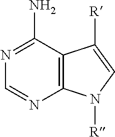 | 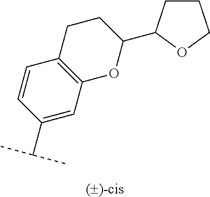 | 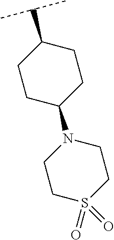 | 39 |
| 66 | 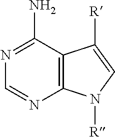 | 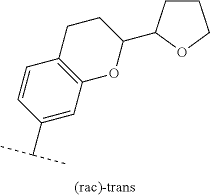 | 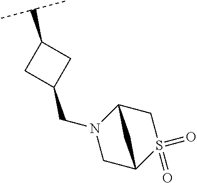 | 24 |
| 67 | 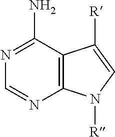 | 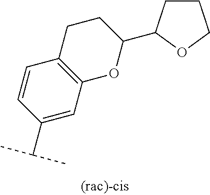 | 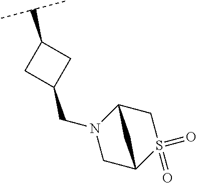 | 17 |
| 68 | 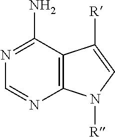 | 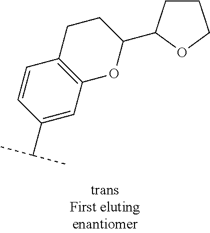 | 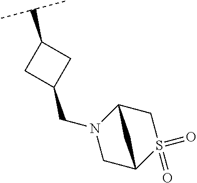 | 9 |
| 69 | 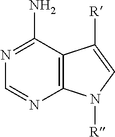 | 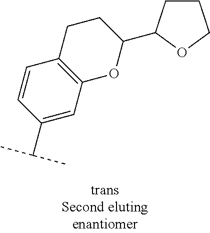 | 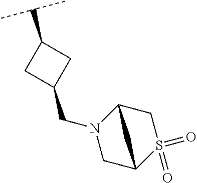 | 362 |
| 70 | 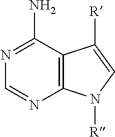 | 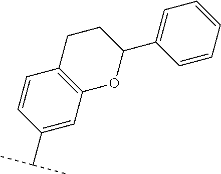 | 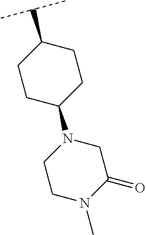 | |

### Enzymatic Kinase Selectivities

| | IC₅₀ values (nM) | | | |
|---|---|---|---|---|
| Kinase | Example 16 | Example 21 | Example 26 | Example 38 |
| IGF1R | 50 | 7.8 | 58 | 17 |
| InsR | 780 | 73 | 1 450 | 100 |
| Aurora A | 4 300 | 4 000 | 8 800 | >10 000 |
| cABLT315 | 8 000 | >10 000 | 4 350 | >10 000 |
| CDK2A | >10 000 | >10 000 | >10 000 | >10 000 |
| cKIT | 3 200 | 770 | >10 000 | 6 000 |
| cMET | >10 000 | >10 000 | >10 000 | >10 000 |
| COT1 | >10 000 | >10 000 | >10 000 | >10 000 |
| EphA4 | >10 000 | >10 000 | >10 000 | >10 000 |
| ERK2 | >10 000 | >10 000 | >10 000 | >10 000 |
| FGFR-4 | >10 000 | >10 000 | 8 900 | >10 000 |
| GSK3beta | >10 000 | >10 000 | >10 000 | >10 000 |
| HER2 | >10 000 | >10 000 | >10 000 | >10 000 |
| JAK2 | >10 000 | >10 000 | >10 000 | >10 000 |
| KDR | 160 | 110 | 250 | 240 |
| LCK | 4 460 | 3 600 | 6 150 | 3 800 |
| MK2 | >10 000 | >10 000 | >10 000 | >10 000 |
| P38a | >10 000 | >10 000 | >10 000 | >10 000 |
| PDGFRa | 1 200 | 770 | >10 000 | >10 000 |
| PKA | >10 000 | >10 000 | >10 000 | >10 000 |
| RET | 360 | 25 | 760 | 410 |
| TYK2 | >10 000 | >10 000 | >10 000 | >10 000 |

### V Pharmaceutical Formulations

### Tablets

Tablets comprising a suitable amount of active ingredient, for example one of the compounds of formula I described in Examples 1 to 88 are prepared in customary manner using for example a composition comprising: active ingredient, wheat starch, lactose, colloidal silicic acid, talc and magnesium stearate. Preparation: The active ingredient is mixed with a portion of the wheat starch, with the lactose and the colloidal silicic acid and the mixture is forced through a sieve. A further portion of the wheat starch is made into a paste, on a water bath, with five times the amount of water and the powder mixture is kneaded with the paste until a slightly plastic mass is obtained. The plastic mass is pressed through a sieve of about 3 mm mesh size and dried, and the resulting dry granules are again forced through a sieve. Then the remainder of the wheat starch, the talc and the magnesium stearate are mixed in and the mixture is compressed to form tablets having a breaking notch.

### Soft Capsules

Soft gelatin capsules comprising each a suitable amount of active ingredient, for example one of the compounds of formula I described in Examples 1 to 88, are prepared in customary manner using for example a composition comprising active ingredient and Lauroglykol.

Preparation: The pulverized active ingredient is suspended in Lauroglykol® (propylene glycol laurate, Gattefossé S.A., Saint Priest, France) and ground in a wet pulverizer to a particle size of approx. 1 to 3 μm. 0.419 g portions of the mixture are then dispensed into soft gelatin capsules using a capsule-filling machine.

1. A compound of formula I 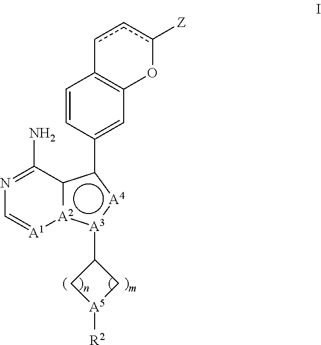
or a salt thereof, wherein [symbol]
represents an optional double or single bond;
A¹ represents N, A² represents C, A³ represents N, A⁴ represents CH or
A¹ represents CH, A² represents N, A³ represents C, A⁴ represents N; and
Z represents aryl, heterocyclic ring A, C₁₋₄alkoxy-C₁₋₄alkyl, or where the valence allows, Z may optionally be a 3, 4, 5 or 6 membered spirocyclic ring C comprising only carbon ring atoms, as shown in formula II wherein y is 1, 2, 3 or 4, and * marks the points of ring fusion in formula I: 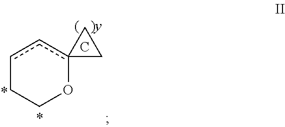
m represents 1 or 2;
n represents 1 or 2;
A⁵-R² represents N—R², N—C(H)R²R³, CR²R³ or CR³—CH₂—R², wherein only a C or a N atom are ring forming atoms, and A⁵ is N—, N—C(H)(R³)—, CR³— or C(R³)—CH₂—;
R³ represents hydrogen, C₁₋₇alkyl, hydroxy;
R² represents heterocyclyl ring B, said heterocyclyl ring B containing from 3 to 12 ring forming atoms, comprising 1, 2, 3 or 4 nitrogen atoms, and comprising 0, 1, 2 or 3 oxygen atoms, and comprising 0, 1, 2 or 3 sulfur atoms,
being saturated, partly saturated, or unsaturated,
being optionally substituted by one to four substituents, the substituents being independently selected from the group consisting of halo, cyano, oxo, hydroxy, amino, nitro, C₁₋₇alkyl, C₁₋₇alkoxy, hydroxy-C₁₋₇alkyl, aminocarbonyl, C₁₋₇alkylaminocarbonyl, di(C₁₋₇alkyl)aminocarbonyl, C₁₋₇alkoxycarbonyl, C₁₋₇alkylcarbonyl; or
R² represents OH, SH, C₁₋₇alkoxy, C₁₋₇alkylthio, amino, C₁₋₇alkylcarbonylamino, C₃₋₇ cycloalkyl-carbonylamino, C₁₋₇alkylsulfonylamino;
or when A⁵-R² represents CR²R³, R² and R³ may join, together with the carbon to which they are attached, to form a 5-membered spirocyclic group, said spirocyclic group comprising 3 carbon ring atoms and 2 ring heteroatoms independently selected from O and N, and wherein said spirocyclic group is substituted at one carbon ring atom with an oxo substituent;
and
heterocyclic ring A is a saturated, partially saturated or unsaturated ring comprising 5, 6, 7, 8, 9 or 10 ring atoms, wherein one or more ring atoms are a heteroatom independently selected from N, O and S, and wherein heterocyclic ring A is optionally substituted with one or two substituents selected from oxo (═O), thiono (═S), imino (═NH), imino-lower alkyl, halogen, amino, N-lower alkylamino, N,N-di-lower alkylamino, N-lower alkanoylamino, N,N-di-lower alkanoylamino, hydroxy, lower alkoxy, lower alkoxy-lower alkoxy, lower alkanoyl, lower alkanoyloxy, cyano, nitro, carboxy, lower alkoxycarbonyl, carbamoyl, amidino, guanidino, ureido, mercapto, and lower alkylthio;

2. The compound of claim 1, or salt thereof, wherein Z represents:
unsubstituted phenyl,
heterocyclic ring A, wherein said heterocyclic ring A is an unsubstituted 5 or 6 membered saturated, partially saturated or unsaturated ring comprising 1 or 2 ring hetero atoms independently selected from N, O and S, preferably N and O,
C₁₋₄alkoxy-C₁₋₄alkyl, or
a 6 membered spirocyclic ring C comprising only carbon ring atoms.

3. The compound of claim 2, or salt thereof, wherein Z represents phenyl or tetrahydrofuranyl.

4. The compound of claim 1, or salt thereof, wherein both m and n represent 1, or both m and n represent 2.

5. The compound of claim 1, or salt thereof, wherein A⁵-R² represents CR²R³ or CR³—CH₂—R².

6. The compound of claim 1, or salt thereof, wherein when m and n are both 1, R² and R³ join, together with the carbon to which they are attached, to form a 5-membered spirocyclic group, said spirocyclic group selected from the spirocyclic rings shown below:

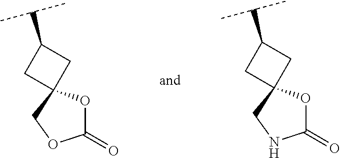

7. The compound of claim 1, or salt thereof, wherein R² is selected from OH, SH, C₁₋₄alkoxy, C₁₋₄alkylthio, amino, C₁₋₄alkylcarbonylamino, C₃₋₆cycloalkyl-carbonylamino and C₁₋₄alkylsulfonylamino, or
R² is heterocyclyl ring B, which is a 5 or 6 membered saturated, partly saturated, or unsaturated ring, wherein the 6-membered saturated ring optionally contains a —CH₂— or —CH₂—CH₂— bridge, and wherein the ring comprises at least 1 N ring atom and
optionally 1, 2 or 3 additional N atoms, or
optionally an additional S atom, said S atom being optionally substituted by 1 or 2=0 substituents,
and wherein heterocyclyl ring B is optionally substituted at a C and/or N atom or atoms with one or two substituents selected from oxo, hydroxy, amino, C₁₋₄alkyl, C₁₋₄alkoxy, hydroxy-C₁₋₄alkyl, aminocarbonyl, C₁₋₄alkylaminocarbonyl, di(C₁₋₄alkyl)aminocarbonyl, C₁₋₄alkoxycarbonyl and C₁₋₄alkylcarbonyl.

8. The compound of claim 7, or salt thereof, wherein R² is selected from OH, NH₂, —NHCOCH₃, —NHSO₂CH₃, —NH—CO-cyclopropyl, —NH—CO-isopropyl, and —NH—CO-methyl, or R² is selected from any one of the following groups, where * indicates the point of attachment:

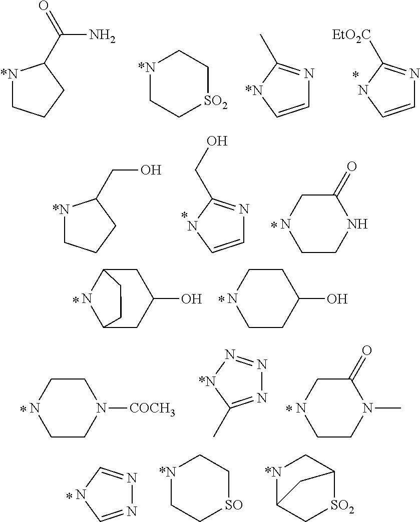

9. The compound of claim 1, or salt thereof, wherein the group attached to A³ is selected from:

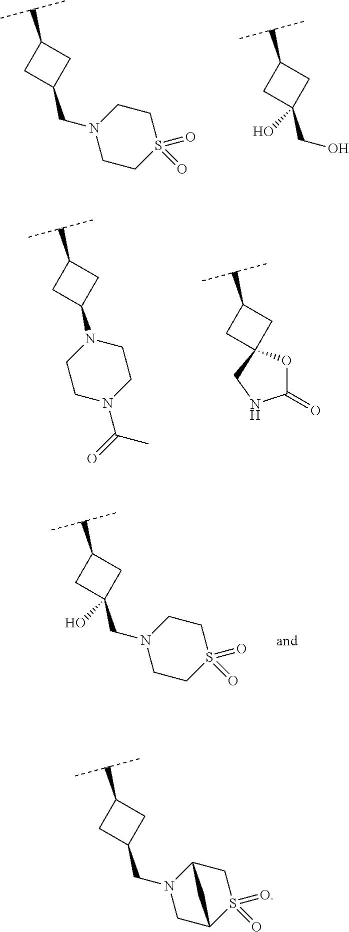

10. The compound of claim 1, or salt thereof, wherein R³ is H, methyl or hydroxy.

11. A compound, or salt thereof, selected from:
(R,S)-{cis-3-[4-Amino-5-(2-phenyl-chroman-7-yl)-pyrrolo[2,3-d]pyrimidin-7-yl]-cyclobutyl}-methanol
(R,S)-{Trans-3-[4-amino-5-(2-phenyl-chroman-7-yl)-pyrrolo[2,3-d]pyrimidin-7-yl]-cyclobutyl}-methanol
(R,S)-{cis-3-[8-Amino-1-(2-phenyl-chroman-7-yl)-imidazo[1,5-a]pyrazin-3-yl]-cyclobutyl}-methanol
1-{cis-3-[8-Amino-1-((R)-2-phenyl-chroman-7-yl)-imidazo[1,5-a]pyrazin-3-yl]-cyclobutylmethyl}-(S)-pyrrolidine-2-carboxylic acid amide
1-{cis-3-[8-amino-1-((S)-2-phenyl-chroman-7-yl)-imidazo[1,5-a]pyrazin-3-yl]-cyclobutylmethyl}-(S)-pyrrolidine-2-carboxylic acid amide
(R,S)—(Z)-3-[8-Amino-1-(2-phenyl-chroman-7-yl)-imidazo[1,5-a]pyrazin-3-yl]-1-hydroxymethyl-cyclobutanol
(R,S)-(E)-3-[8-amino-1-(2-phenyl-chroman-7-yl)-imidazo[1,5-a]pyrazin-3-yl]-1-hydroxymethyl-cyclobutanol
(R,S)-7-[cis-3-(1,1-Dioxo-thiomorpholin-4-ylmethyl)-cyclobutyl]-5-(2-phenyl-chroman-7-yl)-7H-pyrrolo[2,3-d]pyrimidin-4-ylamine
(R,S)-3-[cis-3-(2-Methyl-imidazol-1-ylmethyl)-cyclobutyl]-1-(2-phenyl-chroman-7-yl)-imidazo[1,5-a]pyrazin-8-ylamine
(R,S)-(E)-3-[4-Amino-5-(2-phenyl-chroman-7-yl)-pyrrolo[2,3-d]pyrimidin-7-yl]-1-hydroxymethyl-cyclobutanol
(R,S)—(Z)-3-[4-amino-5-(2-phenyl-chroman-7-yl)-pyrrolo[2,3-d]pyrimidin-7-yl]-1-hydroxymethyl-cyclobutanol
(R,S)-1-{cis-3-[8-Amino-1-(2-phenyl-chroman-7-yl)-imidazo[1,5-a]pyrazin-3-yl]-cyclobutylmethyl}-1H-imidazole-2-carboxylic acid ethyl ester
{cis-3-[4-Amino-5-((S)-2-phenyl-chroman-7-yl)-pyrrolo[2,3-d]pyrimidin-7-yl]-cyclobutyl}-methanol
{cis-3-[4-Amino-5-((R)-2-phenyl-chroman-7-yl)-pyrrolo[2,3-d]pyrimidin-7-yl]-cyclobutyl}-methanol
(R,S)-7-(cis-3-Aminomethyl-cyclobutyl)-5-(2-phenyl-chroman-7-yl)-7-yl)-pyrrolo[2,3-d]pyrimidin-4-ylamine
(1-{cis-3-[4-Amino-5-((R)-2-phenyl-chroman-7-yl)-pyrrolo[2,3-d]pyrimidin-7-yl]-cyclobutylmethyl}-(S)-pyrrolidin-2-yl)-methanol
(1-{cis-3-[4-amino-5-((S)-2-phenyl-chroman-7-yl)-pyrrolo[2,3-d]pyrimidin-7-yl]-cyclobutylmethyl}-(S)-pyrrolidin-2-yl)-methanol
(R,S)-1-{cis-3-[4-Amino-5-(2-phenyl-chroman-7-yl)-pyrrolo[2,3-d]pyrimidin-7-yl]-cyclobutylmethyl}-1H-imidazole-2-carboxylic acid ethyl ester
(R,S)—N-{cis-3-[4-Amino-5-(2-phenyl-chroman-7-yl)-pyrrolo[2,3-d]pyrimidin-7-yl]-cyclobutylmethyl}-acetamide
(R,S)—N-{cis-3-[4-Amino-5-(2-phenyl-chroman-7-yl)-pyrrolo[2,3-d]pyrimidin-7-yl]-cyclobutylmethyl}-methanesulfonamide
(R,S)-(1-{cis-3-[4-Amino-5-(2-phenyl-chroman-7-yl)-pyrrolo[2,3-d]pyrimidin-7-yl]-cyclobutylmethyl}-1H-imidazol-2-yl]-methanol
(R,S)—(Z)-3-[8-Amino-1-(2-phenyl-chroman-7-yl)-imidazo[1,5-a]pyrazin-3-yl]-1-methylcyclobutanol
7-[cis-3-(1,1-Dioxo-1-thiomorpholin-4-ylmethyl)-cyclobutyl]-5-(2-phenyl-4H-chromen-7-yl)-7H-pyrrolo[2,3-d]pyrimidin-4-ylamine
(R,S)-7-[cis-3-(1,1-Dioxo-1-thiomorpholin-4-ylmethyl)-cyclobutyl]-5-(2-phenyl-2H-chromen-7-yl)-7H-pyrrolo[2,3-d]pyrimidin-4-ylamine
(R,S)-{cis-3-[8-Amino-1-(2-phenyl-2H-chromen-7-yl)-imidazo[1,5-a]pyrazin-3-yl]-cyclobutyl}-methanol
(R,S)-{cis-3-[4-Amino-5-(2-phenyl-2H-chromen-7-yl)-pyrrolo[2,3-d]pyrimidin-7-yl]-cyclobutyl}-methanol
(R)-7-[cis-3-(1,1-Dioxo-thiomorpholin-4-ylmethyl)-cyclobutyl]-5-(2-phenyl-chroman-7-yl)-7H-pyrrolo[2,3-d]pyrimidin-4-ylamine
(S)-7-[cis-3-(1,1-dioxo-thiomorpholin-4-ylmethyl)-cyclobutyl]-5-(2-phenyl-chroman-7-yl)-7H-pyrrolo[2,3-d]pyrimidin-4-ylamine
(S)-(E)-3-[4-Amino-5-(2-phenyl-chroman-7-yl)-pyrrolo[2,3-d]pyrimidin-7-yl]-1-hydroxymethyl-cyclobutanol
(R)-(E)-3-[4-amino-5-(2-phenyl-chroman-7-yl)-pyrrolo[2,3-d]pyrimidin-7-yl]-1-hydroxymethyl-cyclobutanol
(R,S)-4-{3-[4-amino-5-(2-phenyl-chroman-7-yl)-pyrrolo[2,3-d]pyrimidin-7-yl]-cyclobutylmethyl}-piperazin-2-one
(E)-3-{4-Amino-5-[2-(tetrahydro-furan-2-yl)-chroman-7-yl]-pyrrolo[2,3-d]pyrimidin-7-yl}-1-hydroxymethyl-cyclobutanol
(I)-7-[cis-3-(1,1-Dioxo-1-thiomorpholin-4-ylmethyl)-cyclobutyl]-5-[2-(tetrahydro-furan-2-yl)-chroman-7-yl]-7H-pyrrolo[2,3-d]pyrimidin-4-ylamine
(u)-7-[3-(1,1-Dioxo-1-thiomorpholin-4-ylmethyl)-cyclobutyl]-5-[2-(tetrahydro-furan-2-yl)-chroman-7-yl]-7H-pyrrolo[2,3-d]pyrimidin-4-ylamine
(I)-(E)-3-{4-Amino-5-[2-(tetrahydro-furan-2-yl)-chroman-7-yl]-pyrrolo[2,3-d]pyrimidin-7-yl}-1-hydroxymethyl-cyclobutanol
(u)-(E)-3-{4-amino-5-[2-(tetrahydro-furan-2-yl)-chroman-7-yl]-pyrrolo[2,3-d]pyrimidin-7-yl}-1-hydroxymethyl-cyclobutanol
(R,R)-(E)-3-{4-Amino-5-[2-(tetrahydro-furan-2-yl)-chroman-7-yl]-pyrrolo[2,3-d]pyrimidin-7-yl}-1-hydroxymethyl-cyclobutanol
(S,S)-(E)-3-{4-amino-5-[2-(tetrahydro-furan-2-yl)-chroman-7-yl]-pyrrolo[2,3-d]pyrimidin-7-yl}-1-hydroxymethyl-cyclobutanol
(R,S)-8-{cis-3-[4-Amino-5-(2-phenyl-chroman-7-yl)-pyrrolo[2,3-d]pyrimidin-7-yl]-cyclobutylmethyl}-8-aza-bicyclo[3.2.1]octan-3-ol
1-{3-[4-Amino-5-(2-phenyl-chroman-7-yl)-pyrrolo[2,3-d]pyrimidin-7-yl]-cyclobutylmethyl}-piperidin-4-ol
1-(4-{cis-3-[4-Amino-5-(2-phenyl-chroman-7-yl)-pyrrolo[2,3-d]pyrimidin-7-yl]-cyclobutyl}-piperazin-1-yl)-ethanone
(1-{cis-3-[8-Amino-1-(2-phenyl-chroman-7-yl)-imidazo[1,5-a]pyrazin-3-yl]-cyclobutylmethyl}-1H-imidazol-2-yl)-methanol
7-[cis-3-(5-Methyl-tetrazol-1-ylmethyl)-cyclobutyl]-5-(2-phenyl-chroman-7-yl)-7H-pyrrolo[2,3-d]pyrimidin-4-ylamine
(Z)-3-[4-Amino-5-(2-phenyl-chroman-7-yl)-pyrrolo[2,3-d]pyrimidin-7-yl]-1-hydroxymethyl-cyclobutanol
[(E)-3-[4-amino-5-(2-phenyl-chroman-7-yl)-pyrrolo[2,3-d]pyrimidin-7-yl]-1-hydroxymethyl-cyclobutanol
(E)-2-[4-Amino-5-(2-phenyl-chroman-7-yl)-pyrrolo[2,3-d]pyrimidin-7-yl]-5,7-dioxa-spiro[3.4]octan-6-one
(E)-2-[4-Amino-5-(2-phenyl-chroman-7-yl)-pyrrolo[2,3-d]pyrimidin-7-yl]-5-oxa-7-aza-spiro[3.4]octan-6-one
(E)-3-[4-Amino-5-(2-phenyl-chroman-7-yl)-pyrrolo[2,3-d]pyrimidin-7-yl]-1-(1,1-dioxothiomorpholin-4-ylmethyl)-cyclobutanol
4-{3-[4-Amino-5-(2-phenyl-chroman-7-yl)-pyrrolo[2,3-d]pyrimidin-7-yl]-cyclobutylmethyl}-1-methyl-piperazin-2-one
(E)-3-[4-Amino-5-(2-phenyl-chroman-7-yl)-pyrrolo[2,3-d]pyrimidin-7-yl]-1-[1,2,4]triazol-4-ylmethylcyclobutanol
cis-3-[4-Amino-5-(2-phenyl-chroman-7-yl)-pyrrolo[2,3-d]pyrimidin-7-yl]-cyclobutanol
trans-3-[4-Amino-5-(2-phenyl-chroman-7-yl)-pyrrolo[2,3-d]pyrimidin-7-yl]-cyclobutanol
5-(2-Ethoxymethyl-chroman-7-yl)-7-[cis-3-(1-oxo-thiomorpholin-4-ylmethyl)-cyclobutyl]-7H-pyrrolo[2,3-d]pyrimidin-4-ylamine
(±)-N-(cis-4-(4-amino-5-(2-phenylchroman-7-yl)-7H-pyrrolo[2,3-d]pyrimidin-7-yl)cyclohexyl)cyclopropanecarboxamide
(±)-N-(cis-4-(4-amino-5-(2-phenylchroman-7-yl)-7H-pyrrolo[2,3-d]pyrimidin-7-yl)cyclohexyl)isobutyramide
7-(cis-3-(1,1-dioxo-thiomorpholin-4-ylmethyl)cyclobutyl)-5-(spiro[chroman-2,1′-cyclohexane]-7-yl)-7H-pyrrolo[2,3-d]pyrimidin-4-amine
(±)-7-(cis-3-(1,1-dioxo-thiomorpholin-4-ylmethyl)cyclobutyl)-5-(2-(pyridin-2-yl)chroman-7-yl)-7H-pyrrolo[2,3-d]pyrimidin-4-amine
(±)-N-(cis-4-(4-amino-5-(2-phenylchroman-7-yl)-7H-pyrrolo[2,3-d]pyrimidin-7-yl)cyclohexyl)acetamide
(±)-(E)-3-(4-amino-5-(2-(pyridin-2-yl)chroman-7-yl)-7H-pyrrolo[2,3-d]pyrimidin-7-yl)-1-(hydroxymethyl)cyclobutanol
(±)-4-((cis-3-(4-amino-5-(2-(pyridin-2-yl)chroman-7-yl)-7H-pyrrolo[2,3-d]pyrimidin-7-yl)cyclobutyl)methyl)-1-methylpiperazin-2-one
(±)-N-((cis-3-(4-amino-5-(2-(pyridin-2-yl)chroman-7-yl)-7H-pyrrolo[2,3-d]pyrimidin-7-yl)cyclobutyl)methyl)acetamide
(±)-N-(cis-4-(4-amino-5-(-2-(trans-tetrahydrofuran-2-yl)chroman-7-yl)-7H-pyrrolo[2,3-d]pyrimidin-7-yl)cyclohexyl)acetamide
(±)-N-(cis-4-(4-amino-5-(-2-(cis-tetrahydrofuran-2-yl)chroman-7-yl)-7H-pyrrolo[2,3-d]pyrimidin-7-yl)cyclohexyl)acetamide
7-[cis-3-((S,S)-2-oxo-2λ6-thia-5-aza-bicyclo[2,2,1]hept-5-ylmethyl)-cyclobutyl]-5-(2-pyridin-2-yl-chroman-7-yl)-7H-pyrrolo[2,3-d]pyrimidin-4-ylamine
7-[cis-3-((S,S)-2,2-Dioxo-2λ6-thia-5-aza-bicyclo[2,2,1]hept-5-ylmethyl)-cyclobutyl]-5-(2-pyridin-2-yl-chroman-7-yl)-7H-pyrrolo[2,3-d]pyrimidin-4-ylamine
(±)-7-(cis-3-(1-oxo-thiomorpholin-4-ylmethyl)cyclobutyl)-5-(2-(pyridin-2-yl)chroman-7-yl)-7H-pyrrolo[2,3-d]pyrimidin-4-amine
(±)-7-[cis-4-(1,1-dioxo-1λ6-thiomorpholin-4-yl)-cyclohexyl]-5-[2-trans-(tetrahydro-furan-2-yl)chroman-7-yl]-7H-pyrrolo[2,3-d]pyrimidin-4-ylamine
(±)-7-[cis-4-(1,1-dioxo-1λ6-thiomorpholin-4-yl)-cyclohexyl]-5-[2-cis-(tetrahydro-furan-2-yl)chroman-7-yl]-7H-pyrrolo[2,3-d]pyrimidin-4-ylamine
7-[cis-3-((S,S)-2,2-dioxo-2λ6-thia-5-aza-bicyclo[2,2,1]hept-5-ylmethyl)-cyclobutyl]-5-[2-trans-(tetrahydro-furan-2-yl-chroman-7-yl)-7H-pyrrolo[2,3-d]pyrimidin-4-ylamine
7-[cis-3-((S,S)-2,2-dioxo-2λ6-thia-5-aza-bicyclo[2,2,1]hept-5-ylmethyl)-cyclobutyl]-5-[2-cis-(tetrahydro-furan-2-yl-chroman-7-yl)-7H-pyrrolo[2,3-d]pyrimidin-4-ylamine
7-[cis-3-((S,S)-2,2-dioxo-2λ6-thia-5-aza-bicyclo[2,2,1]hept-5-ylmethyl)-cyclobutyl]-5-[(R,R)-2-(tetrahydro-furan-2-yl-chroman-7-yl)-7H-pyrrolo[2,3-d]pyrimidin-4-ylamine
7-[cis-3-((S,S)-2,2-dioxo-2λ6-thia-5-aza-bicyclo[2,2,1]hept-5-ylmethyl)-cyclobutyl]-5-[(S,S)-2-(tetrahydro-furan-2-yl-chroman-7-yl)-7H-pyrrolo[2,3-d]pyrimidin-4-ylamine and
(±)-4-(cis-4-(4-amino-5-(2-phenylchroman-7-yl)-7H-pyrrolo[2,3-d]pyrimidin-7-yl)cyclohexyl)-1-methylpiperazin-2-one

12. A pharmaceutical composition comprising a compound of claim 1, and one or more pharmaceutically acceptable carriers.

13. A combination comprising a compound of claim 1, and one or more therapeutically active agents, selected from antiproliferative agent or agents.

14-17. (canceled)

18. A method for the treatment of an IGF-1R mediated disorder or disease selected from multiple myeloma, neuroblastoma, synovial, hepatocellular, Ewing's Sar-coma, adrenocotical carcinoma (ACC) or a solid tumor selected from osteosarcoma, mela-noma, tumor of breast, renal, prostate, colorectal, thyroid, ovarian, pancreatic, lung, uterine or gastrointestinal tumor, acute lung injury, pulmonary fibrosis and diabetic retinopathy, or a method of modulating IGF-1R activity in a subject, comprising the step of administering to a subject a therapeutically effective amount of a compound of claim 1.
